SEQUENCE LISTING (2 sequences)

SEQ ID NO: 1 - DNA, 12 bp, Artificial Sequence
  Synthetic Construct
tatggtcgtcga

SEQ ID NO: 2 - DNA, 12 bp, Artificial Sequence
  Synthetic Construct
tatggtcatcga

(19) United States
(12) Patent Application Publication
(10) Pub. No.: US 2024/0018599 A1
(43) Pub. Date: Jan. 18, 2024

(54) METHODS AND SYSTEMS FOR DETECTING RESIDUAL DISEASE

(71) Applicant: Ultima Genomics, Inc., Newark, CA (US)

(72) Inventors: Omer BARAD, Mazkeret Batya (IL); Itai RUSINEK, Holon (IL); Ilya SOIFER, Tel Aviv (IL)

(21) Appl. No.: 18/035,075

(22) PCT Filed: Nov. 17, 2021
(86) PCT No.: PCT/US2021/072476

§ 371 Date: May 2, 2023

Related U.S. Application Data
(60) Provisional application No. 63/115,425, filed on Nov. 18, 2020.

(51) Int. Cl. C12Q 1/6886; C12Q 1/6827

(52) U.S. Cl. CPC C12Q 1/6886; C12Q 1/6827; C12Q 2600/112

(57) ABSTRACT

Described herein are methods, devices, and systems for measuring a level, presence, recurrence, progression, or regression of a disease (such as cancer), for example a fraction of nucleic acid molecules (such as cell-free DNA) in a sample from an individual that relate to diseased tissue (such as cancer tissue). The methods include generating, using the sequencing data comprising sequencing reads associated with loci selected from a personalized disease-associated small nucleotide variant panel, a plurality of variant motif-specific models that each associate sequencing data corresponding to a respective variant motif, a background factor indicative of a false positive error rate for the respective variant motif, and an estimated fraction of the nucleic acid molecules associated with the disease. From the plurality of variant motif-specific models, a fraction of the nucleic acid molecules associated with the disease for the individual can be determined.

### CROSS-REFERENCE TO RELATED APPLICATION

This application claims priority to and the benefit of U.S. Provisional Application No. 63/115,425, filed Nov. 18, 2020, which is incorporated herein by reference for all purposes.

### FIELD OF THE INVENTION

Described herein are methods, systems, and devices for measuring a fraction of nucleic acid molecules in a sample associated with a disease, such as cancer, using nucleic acid sequencing data. Also described are methods, systems, and devices for measuring a level of, a presence, a recurrence, a progression, or a regression of a disease, such as cancer.

### BACKGROUND

Detection and quantification of residual disease before, during and after cancer treatment can be used to monitor the effectiveness of cancer treatment or cancer remission in a patient. Targeted nucleic acid sequencing methods have been previously used to determine differences (i.e., variants) between disease-free tissue and cancerous tissue. Targeted sequencing methods often look for mutations in known driver genes or known mutational hotspots within the cancer genome or exome, or employ deep sequencing methods to ensure accurate variant calls at specific targeted loci.

The amount of cell-free DNA (“cfDNA”) originating from tumors (also referred to as “circulating tumor DNA” or “ctDNA”) in an individual can correlate with the severity of the disease. Other than for the most progressed diseases states, only a small fraction of DNA in a sample originates from diseased tissue, with the vast majority of DNA coming from non-diseased tissue in the individual. This makes accurate measurements of the amount of cfDNA originating from diseased tissue particularly challenging. Current approaches often involve very high sensitivity schemes, such as custom qPCR or custom enrichment, targeting relatively few cancer-specific variants.

### BRIEF SUMMARY OF THE INVENTION

Described herein are methods, systems, and devices for measuring a level of a disease (such as cancer) in an individual, as well as methods for measuring a presence, recurrence, progression, or regression of a disease (such as cancer) in an individual. The methods include determining a fraction of nucleic acid molecules in a fluidic sample from the individual that are associated with the disease, thereby indicating the level of disease in the individual. Background noise can limit detection of the disease fraction in previous methods. As further described herein, the background noise (i.e., false positives) is not uniform, but can depend on the variant motif of the disease associated variants used to determine the fraction. This background noise can be accounted for using variant motif-specific models that each associate sequencing data corresponding to a respective variant motif, a background factor indicative of a false positive error rate for the respective variant motif, and an estimated fraction of the nucleic acid molecules associated with the disease.

A method of determining a level of a disease (e.g., cancer, such as metastatic cancer) in an individual can include: obtaining sequencing data for nucleic acid molecules (e.g., cfDNA molecules) obtained from a fluidic sample (e.g., a blood sample, a plasma sample, a saliva sample, a urine sample, or a fecal sample) from the individual, the sequencing data comprising sequencing reads associated with loci selected from a personalized disease-associated small nucleotide variant panel; generating, using the sequencing data, a plurality of variant motif-specific models that each associate sequencing data corresponding to a respective variant motif, a background factor indicative of a false positive error rate for the respective variant motif, and an estimated fraction of the nucleic acid molecules associated with the disease; and determining, from the plurality of variant motif-specific models, a fraction (e.g., a tumor fraction) of the nucleic acid molecules associated with the disease for the individual, wherein the fraction indicates the level of the disease in the individual. The level of the disease may be a presence or absence of the disease, or it may be a quantitative value indicating the severity of the disease.

A method of determining a presence or absence of a disease (e.g., cancer, such as metastatic cancer) in an individual can include obtaining sequencing data for nucleic acid molecules (e.g., cfDNA molecules) obtained from a fluidic sample (e.g., a blood sample, a plasma sample, a saliva sample, a urine sample, or a fecal sample) from the individual, the sequencing data comprising sequencing reads associated with loci selected from a personalized disease-associated small nucleotide variant panel; generating, using the sequencing data, a plurality of variant motif-specific models that each associate sequencing data corresponding to a respective variant motif, a background factor indicative of a false positive error rate for the respective variant motif, and an estimated fraction of the nucleic acid molecules associated with the disease; and determining, from the plurality of variant motif-specific models, a fraction (e.g., a tumor fraction) of the nucleic acid molecules associated with the disease for the individual; and comparing the fraction to a background level, wherein the fraction being above the background level indicates the presence of the disease in the individual. The method may further include determining whether the difference between the determined fraction for the individual is greater than a background level with statistical significance.

For example, the method may further include detecting a recurrence of the disease. In some examples, the method further includes measuring a progression or regression of the disease by comparing the measured level of the disease to a previously measured level of the disease. The progression or regression of the disease may be based on a statistically significant change in the measured level of the disease.

The sequencing data for the above methods can be generated by sequencing the nucleic acid molecules using non-terminating nucleotides provided in separate nucleotide flows according to a flow-cycle order comprising a plurality of flow positions, wherein the flow positions correspond to the nucleotide flows. For example, the step of obtaining the sequencing data can include sequencing the nucleic acid molecules using non-terminating nucleotides provided in separate nucleotide flows according to a flow-cycle order comprising a plurality of flow positions, wherein the flow positions correspond to the nucleotide flows. The sequencing data may be untargeted sequencing data, such as sequencing data is obtained from an untargeted whole genome. The mean sequencing depth of the sequencing data may be at least 0.01 and/or less than about 100 (e.g., less than about 10, or less than about 1). The sequencing data may be obtained, for example, using surface-based sequencing of nucleic acid molecules, and wherein the nucleic acid molecules are not amplified prior to attaching the nucleic acid molecules to a surface. Optionally, the sequencing data is obtained without the use of unique molecular identifiers (UMIs) and/or without the use of sample identification barcodes.

The background factor may be based on sequencing data for nucleic acid molecules obtained from a plurality of control individuals, for example sequencing data that includes sequencing reads associated with loci selected from the personalized disease-associated small nucleotide variant panel. Optionally, the sequencing data for the nucleic acid molecules of the individual and the sequencing data for the nucleic acid molecules for the plurality of control individuals are simultaneously obtained in a pooled sample.

The small nucleotide variant panel may be filtered, for example, such that at least 90% of small nucleotide variants in the personalized disease-associated small nucleotide variant panel are associated with small nucleotide variant sequencing data that differs from reference sequencing data associated with a reference sequence at two or more flow positions when the small nucleotide variant sequencing data and the reference sequencing data are sequenced using non-terminating nucleotides provided in separate nucleotide flows according to a flow-cycle order comprising a plurality of flow positions, wherein the flow positions correspond to nucleotide flows. A more stringent approach may also be taken, for example, wherein at least 90% of small nucleotide variants in the personalized disease-associated small nucleotide variant panel are associated with small nucleotide variant sequencing data that differs from reference sequencing data associated with a reference sequence across one or more flow cycles when the sequencing data and the reference sequencing data are sequenced using non-terminating nucleotides provided in separate nucleotide flows according to the flow-cycle order.

The sequencing reads in the sequencing data and/or the small nucleotide variants in the personalized disease-associated small nucleotide variant panel can be filtered to limit false positives in the sequencing data. For example, the sequencing reads may be characterized as an alternate read, a reference read, or an ambiguous read, wherein a sequencing read characterized as an ambiguous read is excluded from the plurality of variant motif-specific models. Additionally or alternatively, for each of the sequencing reads, a likelihood that the sequencing read corresponds to a variant sequence and a likelihood that the sequencing read corresponds to a reference sequence ca be determined, and for a respective sequence read, when the difference between the likelihood that the respective sequencing read corresponds to an alternative sequence and the likelihood that the respective sequencing read corresponds to a reference sequence is less than a predetermined likelihood difference threshold (e.g., a threshold set at a value of 5 orders of magnitude or higher), sequencing data corresponding to the respective sequencing read can be excluded from the plurality of variant motif-specific models.

The plurality of variant motif-specific models can include, for example, a respective variant motif-specific model for each of a plurality of trinucleotide SNP motifs. For example, the plurality of variant motif-specific models can include 192 trinucleotide SNP variant motif-specific models.

Each variant motif-specific model can associate the sequencing data corresponding to variant motif, m, to the background factor, BGₘ, and the estimated faction, F, according to:

Nₘ^{alt}=(F+BGₘ)Nₘ^{total}

wherein Nₘ^{alt} is a number of alternative sequencing reads comprising a locus corresponding to variant motif m and Nₘ^{total} is a total number of sequencing reads comprising a locus corresponding to variant motif m.

Each variant motif-specific model may be a binomial distribution of the sequencing reads comprising a locus corresponding to variant motif m, with a probability, pₘ, of observing an alternative sequencing read comprising a locus corresponding to variant motif m based on pₘ=F+BGₘ, wherein F is the estimated fraction, and BGₘ is the background factor. Determining the fraction for the individual can include determining a maximum likelihood estimate for the fraction given the plurality of variant motif-specific models.

Determining the fraction for the individual can include determining, for each variant motif-specific model, a statistical value indicative of a likelihood of each of a plurality of estimated fractions, given the sequencing data for the nucleic acid molecules obtained from the fluidic sample from the individual corresponding to the variant motif, and determining a most likely fraction given the statistical values for each variant motif. Each variant motif-specific model may include a plurality of binomial distributions of sequencing reads comprising a locus corresponding to variant motif m, with each binomial distribution having a probability of a sequencing read being an alternate read equal to an estimated fraction selected from the plurality of estimated fractions.

The fraction for the individual can be determined by determining, for each variant motif-specific model, a statistical value indicative of a likelihood of each of a plurality of estimated fractions, given the sequencing data for the nucleic acid molecules obtained from the fluidic sample from the individual corresponding to the variant motif and control sequencing data for nucleic acid molecules obtained from one or more control fluidic samples (e.g., a plurality of control fluidic samples) corresponding to the variant motif, wherein the control sequencing data is adjusted for one or more non-zero estimated fractions; and determining a most likely fraction given the statistical values for each variant motif. Each statistical value can be determined using an exact test (e.g., Fisher's exact test). The control sequencing data can be adjusted for each of the one or more non-zero estimated fractions using a random realization method with a distribution probability equal to the respective non-zero estimated fraction. For example, for each estimated tumor fraction, the statistical value indicative of the likelihood can be an average of a plurality of likelihood values obtained using a plurality of random realizations, each with a distribution probability equal to the respective non-zero estimated fraction.

The method can include generating the personalized disease-associated small nucleotide variant panel. Small nucleotide variants other than single nucleotide polymorphisms (SNPs) may be excluded from the personalized disease-associated small nucleotide variant panel. The panel may include, for example, 300 or more small nucleotide variant loci. The disease-associated small nucleotide variant panel may include passenger mutations and/or driver mutations. The personalized disease-associated small nucleotide variant panel can include small nucleotide variants detected from sequencing data for nucleic acid molecules derived from a diseased tissue sample (e.g., a tumor biopsy sample obtained from the individual). Nucleic acid molecules derived from a diseased tissue sample obtained from the individual may be sequenced to obtain diseased tissue sequencing data, and small nucleotide variants that have a variant allele fraction in the nucleic acid molecules derived from the diseased tissue sample lower than a predetermined low-fraction threshold may be excluded from the personalized disease-associated small nucleotide variant panel. Additionally or alternatively, small nucleotide variants that have a variant allele fraction in the nucleic acid molecules derived from the diseased tissue sample higher than a predetermined high-fraction threshold may be excluded from the personalized disease-associated small nucleotide variant panel.

Small nucleotide variants characterized as likely germline variants or likely non-disease related somatic variants may be excluded from the personalized disease-associated small nucleotide variant panel. Nucleic acid molecules derived from a non-diseased tissue sample (e.g., a tissue comprising white blood cells or peripheral blood mononuclear cells, for example a buffy coat) obtained from the individual may be sequenced to obtain non-diseased tissue sequencing data, and the method further comprises excluding, from the personalized disease-associated small nucleotide variant panel, small nucleotide variants at loci that have no sequencing coverage within the non-diseased tissue sequencing data.

Optionally, small nucleotide variants present in a general population of individuals at an allele frequency greater than a predetermined allele threshold (e.g., about 0.01) may be excluded from the personalized disease-associated small nucleotide variant panel. Optionally, small nucleotide variants at loci with two or more non-reference alleles may be excluded from the personalized disease-associated small nucleotide variant panel. Optionally, small nucleotide variants within a low complexity region may be excluded from the personalized disease-associated small nucleotide variant panel. Optionally, small nucleotide variants at loci associated with a predetermined number or proportion of sequencing reads that have a mapping quality score below a predetermined mapping quality threshold may be excluded from the personalized disease-associated small nucleotide variant panel. Optionally, small nucleotide variants at loci that have a bias for reference reads or alternate reads may be excluded from the personalized disease-associated small nucleotide variant panel.

The method may further include identifying one or more outlier small nucleotide variants within the personalized disease-associated small nucleotide variant panel that are associated with a locus-specific fraction outlier, given the sequencing data for nucleic acid molecules obtained from a fluidic sample from the individual, and excluding sequencing data associated with said one or more outlier small nucleotide variants from the plurality of variant motif-specific models.

For any of the methods described above, the method can include generating a report that indicates the presence, absence, or level of disease in the individual. Optionally, the report is provided to a patient or a healthcare representative of the patient.

Also provided herein is a system that includes one or more processors and a non-transitory computer-readable medium that stores one or more programs comprising instructions for implementing any of the above methods.

### BRIEF DESCRIPTION OF THE DRAWINGS

FIG. 1A shows sequencing data obtained by extending a primer with a sequence of TATGGTCGTCGA (SEQ ID NO: 1) using a repeated flow-cycle order of T-A-C-G. The sequencing data is representative of the extended primer strand, and sequencing information for the complementary template strand can be readily determined is effectively equivalent.

FIG. 1B shows the sequencing data shown in FIG. 1A with the most likely sequence, given the sequencing data, selected based on the highest likelihood at each flow position (as indicated by stars).

FIG. 1C shows the sequencing data shown in FIG. 1A with traces representing two different candidate sequences: TATGGTCATCGA (SEQ ID NO: 2) (closed circles) and TATGGTCGTCGA (SEQ ID NO: 1) (open circles). The likelihood that the sequencing data matches a given sequence can be determined as the product of the likelihood that each flow position matches the candidate sequence. The first candidate sequence (SEQ ID NO: 2) may also be considered an exemplary reference sequence reverse complement, and the second candidate sequence (SEQ ID NO: 1) may be considered an small nucleotide variant-containing sequence, in some embodiments.

FIG. 1D shows the sequencing data for a nucleic acid molecule containing an small nucleotide variant (SEQ ID NO: 1) obtained using a A-G-C-T sequencing cycle and compared to a reference sequence (SEQ ID NO: 2).

FIG. 2 shows an exemplary method of measuring a level of disease (e.g., a tumor) in an individual.

FIG. 3 shows an exemplary method of detecting the presence or absence of a disease (e.g., a tumor) in an individual.

FIG. 4 illustrates an example of a computing device in accordance with some instances, which may be used to implement a method as described herein.

FIG. 5 shows measured Tumor Fraction (TF) for both case (diagonal) and control samples without using motif-specific models. The row indicates the FFPE signature and the column the cfDNA sample.

FIG. 6 shows measured Tumor Fraction (TF) for both case (diagonal) and control samples, using MLE estimation accounting for background using variant motif-specific models.

FIG. 7 shows log-likelihood profiles determined using a Fisher Exact Test method, for patient ABS1405-20 case (right) and controls (left). The log-likelihood for various TF values was calculated, and the maximum likelihood value determined. Additionally, the likelihood for TF=0 was determined, and the significance of the detected optima evaluated by calculating the Z-Score compared to the likelihood at TF=0.

FIG. 8 shows measured Tumor Fraction (TF) for both case (diagonal) and control samples, using the Fisher method accounting for background using variant motif-specific models.

FIG. 9 shows random downsamples of sequencing data from a subject, which provides estimates for detection limits as a function of coverage.

FIG. 10 illustrates an exemplary flowchart method of measuring a level of disease (e.g., a tumor) or detecting the presence or absence of a disease (e.g., a tumor) in an individual, in accordance with some implementations.

FIG. 11 illustrates an exemplary flowchart method of measuring a level of disease (e.g., a tumor) or detecting the presence or absence of a disease (e.g., a tumor) in an individual, in accordance with some implementations.

FIG. 12A shows an example block diagram illustrating a computing device in accordance with some implementations.

FIG. 12B shows an example block diagram illustrating a computing device in accordance with some implementations.

### DETAILED DESCRIPTION OF THE INVENTION

The methods, devices, and systems described herein relate to detecting and/or measuring a level of a disease in an individual. The level of the disease may be a presence or absence of the disease, or it may be a quantitative value indicating the severity of the disease. The level of the disease can be associated with a fraction of nucleic acid molecules (such as cell-free DNA) in a sample that originate from diseased tissue (such as cancer tissue). The disease can be detected or the level measured, for example, by measuring a signal indicative of the rate of detecting small nucleotide variant reads in nucleic acid molecules at selected loci originating from diseased tissue. The detected fraction of nucleic acid molecules in the sample that are associated with the diseased tissue can inform the level of disease in the individual. By detecting the level of disease in the individual, recurrence of a previously present disease (or a disease previously believed to be in remission) can be determined, as can a progression or regression of the disease state.

False positive sequencing errors cause noise that can challenge the accuracy or limit of detection of the measured fraction, particularly when the fraction is close to the noise level. Accounting for the background noise can improve the limit of detection of the disease fraction of nucleic acid molecules. See, for example, PCT/US2020/033217, the contents of which are incorporated herein by reference. Additionally, the background noise can differ between different variant motifs. For example, different variant motifs may have different false positive sequencing errors when the nucleic acid molecules are sequenced using flow sequencing methods, which include sequencing the nucleic acid molecules using non-terminating nucleotides provided in separate nucleotide flows according to a flow-cycle order comprising a plurality of flow positions, wherein the flow positions correspond to the nucleotide flows. It has been discovered that accounting for the background noise on a variant motif-specific basis can significantly improve the limit of detects.

Certain diseased tissue, and in particular cancer, can include thousands (or tens of thousands, hundreds of thousands, or more) mutations throughout the diseased genome, compared to the normal healthy genome of an individual. These mutations may be driver mutations, which confer a growth advantage (e.g., proliferation or survival) to a cancer, or may be passenger mutations, which can be found throughout the coding or non-coding region of the genome but are not believed to confer any growth advantage. In some cases, the passenger mutations accumulated in the cell that became cancerous before becoming cancerous, as even healthy tissue has a certain mutation rate. The broad spectrum of mutations for any given disease in a patient is unique to the patient and to even the particular diseased tissue clone or sub-clone, thus giving the diseased tissue a unique genetic signature. A personalized disease-associated small nucleotide variant panel can be established for the diseased tissue by comparing the genome (or a portion thereof) of the diseased tissue to the genome (or corresponding genome) of the non-diseased tissue of the same patient. Optionally, a subset of the loci from the panel can be selected for analysis, and the selection may be based on, for example, the false positive error rate at a given locus, e.g., being lower than for other loci. The small nucleotide variant panel can comprise passenger mutations and/or driver mutations.

The level of a disease or residual disease (e.g., cancer) in an individual can be measured by a) obtaining sequencing data for nucleic acid molecules obtained from a fluidic sample from the individual, the sequencing data comprising sequencing reads associated with loci selected from a personalized disease-associated small nucleotide variant panel; b) generating, using the sequencing data, i) a plurality of variant motif-specific models that each associate sequencing data corresponding to a respective variant motif, ii) a background factor indicative of a false positive error rate for the respective variant motif, and iii) an estimated fraction of the nucleic acid molecules associated with the disease; and c) determining, from the plurality of variant motif-specific models, a fraction of the nucleic acid molecules associated with the disease for the individual, wherein the fraction indicates the level of the disease in the individual.

By considering the variant motif-specific false positive error rate when measuring a diseased fraction of nucleic acid molecules or a level of the disease in the patient, the overall sequencing depth can be reduced, providing significant time and cost savings. False positive errors can arise due to chemical damage, incorrect base incorporation, or fluorescent read error during sequencing, and can falsely indicate a small nucleotide variant exists at a given locus. To guard against potential false errors at a specific locus, other disease detection methods often require multiple independent small nucleotide variant calls at a given locus, which can only be obtained by sequencing that locus at a depth inversely proportional to the fraction of diseased nucleic acid in the sample. In some cases, other methods involve determining a consensus sequence at a locus from a plurality of sequencing reads. The deep sequencing utilized by other methods generally requires targeting specific loci or a narrow subset of the genome (e.g., mutational hotspots or whole exome sequencing). Additionally, other sequencing methods often require amplification of the nucleic acid molecules during library preparation to independently sequence multiple copies of the same nucleic acid molecule. This amplification process risks introducing additional false errors.

Instead of being concerned with false positive errors at any particular locus, the described methods measure the fraction of diseased nucleic acid molecules or the level of the disease using a variant motif-specific false positive error rate for loci selected for analysis associated with the variant motif. Once the loci have been selected, a false positive at any specific locus does not significantly affect the measurement. Thus, although the loci selected for analysis may be selected using a false positive error rate at each specific locus, the impact of any specific error that may arise from sequencing at a given locus is not considered.

### Definitions

As used herein, the singular forms “a,” “an,” and “the” include the plural reference unless the context clearly dictates otherwise.

Reference to “about” a value or parameter herein includes (and describes) variations that are directed to that value or parameter per se. For example, description referring to “about X” includes description of “X”.

The terms “individual,” “patient,” and “subject” are used synonymously herein, and refer to an animal including a human. A subject generally refers to an individual from whom a biological sample is obtained. The subject may be a mammal or non-mammal. The subject may be an animal, such as a monkey, dog, cat, bird, or rodent. The subject may be a human. The subject may be a patient. The subject may be displaying a symptom of a disease. The subject may be asymptomatic. The subject may be undergoing treatment. The subject may not be undergoing treatment. The subject can have or be suspected of having a disease, such as cancer (e.g., breast cancer, colorectal cancer, brain cancer, leukemia, lung cancer, skin cancer, liver cancer, pancreatic cancer, lymphoma, esophageal cancer, or cervical cancer) or an infectious disease. The subject can have or be suspected of having a genetic disorder such as achondroplasia, alpha-1 antitrypsin deficiency, antiphospholipid syndrome, autism, autosomal dominant polycystic kidney disease, Charcot-Marie-tooth, cri du chat, Crohn's disease, cystic fibrosis, Dercum disease, down syndrome, Duane syndrome, Duchenne muscular dystrophy, factor V Leiden thrombophilia, familial hypercholesterolemia, familial Mediterranean fever, fragile x syndrome, Gaucher disease, hemochromatosis, hemophilia, holoprosencephaly, Huntington's disease, Klinefelter syndrome, Marfan syndrome, myotonic dystrophy, neurofibromatosis, Noonan syndrome, osteogenesis imperfecta, Parkinson's disease, phenylketonuria, Poland anomaly, porphyria, progeria, retinitis pigmentosa, severe combined immunodeficiency, sickle cell disease, spinal muscular atrophy, Tay-Sachs, thalassemia, trimethylaminuria, Turner syndrome, velocardiofacial syndrome, WAGR syndrome, or Wilson disease.

The term “flow order,” as used herein, refers to the order of separate nucleotide flows used to sequence a nucleic acid molecule using non-terminating nucleotides. The flow order may be divided into cycles of repeating units, and the flow order of the repeating units is termed a “flow-cycle order.” A “flow position” refers to the sequential position of a given separate nucleotide flow during the sequencing process.

The term “label,” as used herein, refers to a detectable moiety that is coupled to or may be coupled to another moiety, for example, a nucleotide or nucleotide analog. The label can emit a signal or alter a signal delivered to the label so that the presence or absence of the label can be detected. In some cases, coupling may be via a linker, which may be cleavable, such as photo-cleavable (e.g., cleavable under ultra-violet light), chemically-cleavable (e.g., via a reducing agent, such as dithiothreitol (DTT), tris(2-carboxyethyl)phosphine (TCEP)) or enzymatically cleavable (e.g., via an esterase, lipase, peptidase, or protease). In some instances, the label is a fluorophore.

The term “non-terminating nucleotide,” as used herein, refers to a nucleic acid moiety that can be attached to a 3′ end of a polynucleotide using a polymerase or transcriptase, and that can have another non-terminating nucleic acid attached to it using a polymerase or transcriptase without the need to remove a protecting group or reversible terminator from the nucleotide. Naturally occurring nucleic acids are a type of non-terminating nucleic acid. Non-terminating nucleic acids may be labeled or unlabeled.

The terms “nucleic acid,” “nucleic acid molecule,” “nucleic acid sequence,” “nucleic acid fragment,” “oligonucleotide” and “polynucleotide,” as used herein, generally refer to a polynucleotide that may have various lengths, such as either deoxyribonucleotides or deoxyribonucleic acids (DNA) or ribonucleotides or ribonucleic acids (RNA), or analogs thereof. Non-limiting examples of nucleic acids include DNA, RNA, genomic DNA or synthetic DNA/RNA or coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant nucleic acids, branched nucleic acids, plasmids, vectors, isolated DNA of any sequence, and isolated RNA of any sequence. A nucleic acid molecule can have a length of at least about 10 nucleic acid bases (“bases”), 20 bases, 30 bases, 40 bases, 50 bases, 100 bases, 200 bases, 300 bases, 400 bases, 500 bases, 1 kilobase (kb), 2 kb, 3, kb, 4 kb, 5 kb, 10 kb, 20 kb, 30 kb, 40 kb, 50 kb, 100 kb, 200 kb, 300 kb, 400 kb, 500 kb, 1 megabase (Mb), or more. A nucleic acid molecule (e.g., polynucleotide) can comprise a sequence of four natural nucleotide bases: adenine (A); cytosine (C); guanine (G); and thymine (T) (uracil (U) for thymine (T) when the polynucleotide is RNA). A nucleic acid molecule may include one or more nonstandard nucleotide(s), nucleotide analog(s) and/or modified nucleotide(s).

The term “nucleotide,” as used herein, generally refers to any nucleotide or nucleotide analog. The nucleotide may be naturally occurring or non-naturally occurring. The nucleotide analog may be a modified, synthesized or engineered nucleotide. The nucleotide analog may not be naturally occurring or may include a non-canonical base. The naturally occurring nucleotide may include a canonical base. The nucleotide analog may include a modified polyphosphate chain (e.g., triphosphate coupled to a fluorophore). The nucleotide analog may comprise a label. The nucleotide analog may be terminated (e.g., reversibly terminated). The nucleotide analog may comprise an alternative base.

Nonstandard nucleotides, nucleotide analogs, and/or modified analogs may include, but are not limited to, diaminopurine, 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid(v), 5-methyl-2-thiouracil, 3-(3-amino-3-N-2-carboxypropyl) uracil, (acp3)w, 2,6-diaminopurine, ethynyl nucleotide bases, 1-propynyl nucleotide bases, azido nucleotide bases, phosphoroselenoate nucleic acids and the like. In some cases, nucleotides may include modifications in their phosphate moieties, including modifications to a triphosphate moiety. Additional, non-limiting examples of modifications include phosphate chains of greater length (e.g., a phosphate chain having, 4, 5, 6, 7, 8, 9, 10 or more phosphate moieties), modifications with thiol moieties (e.g., alpha-thio triphosphate and beta-thiotriphosphates) or modifications with selenium moieties (e.g., phosphoroselenoate nucleic acids). Nucleic acid molecules may also be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone. Nucleic acid molecules may also contain amine-modified groups, such as aminoallyl-dUTP (aa-dUTP) and aminohexhylacrylamide-dCTP (aha-dCTP) to allow covalent attachment of amine reactive moieties, such as N-hydroxysuccinimide esters (NHS). Alternatives to standard DNA base pairs or RNA base pairs in the oligonucleotides of the present disclosure can provide higher density in bits per cubic mm, higher safety (resistant to accidental or purposeful synthesis of natural toxins), easier discrimination in photo-programmed polymerases, or lower secondary structure. Nucleotide analogs may be capable of reacting or bonding with detectable moieties for nucleotide detection.

The term “biological sample,” as used herein, generally refers to any sample from a subject or specimen from a subject. The biological sample can be a fluid or tissue from the subject or specimen. The term “tissue” as used herein refers to any cellular material, and can include circulating cells or non-circulating cells. The fluid can be blood (e.g., whole blood), saliva, urine, or sweat. The tissue can be from an organ (e.g., liver, lung, or thyroid), or a mass of cellular material, such as, for example, a tumor. The biological sample can be a feces sample, collection of cells (e.g., cheek swab), or hair sample. The biological sample can be a cell-free or cellular sample. Examples of biological samples include nucleic acid molecules, amino acids, polypeptides, proteins, carbohydrates, fats, or viruses. In an example, a biological sample is a nucleic acid sample including one or more nucleic acid molecules, such as deoxyribonucleic acid (DNA) and/or ribonucleic acid (RNA). The nucleic acid molecules may be cell-free or cell-free nucleic acid molecules, such as cell free DNA or cell free RNA. The nucleic acid molecules may be derived from a variety of sources including human, mammal, non-human mammal, ape, monkey, chimpanzee, reptilian, amphibian, avian, or plant sources. Further, samples may be extracted from variety of animal fluids containing cell free sequences, including but not limited to blood, serum, plasma, vitreous, sputum, urine, tears, perspiration, saliva, semen, mucosal excretions, mucus, spinal fluid, amniotic fluid, lymph fluid and the like. Cell free polynucleotides may be fetal in origin (via fluid taken from a pregnant subject) or may be derived from tissue of the subject itself.

The term “short genetic variant,” as used herein, is used to describe a genetic polymorph (i.e., mutation) that is 10 consecutive bases in length or less (i.e., 10, 9, 8, 7, 6, 5, 4, 3, 2, or 1 base(s) in length). The term includes single nucleotide polymorphisms (SNPs), multi-nucleotide polymorphisms (MNPs), and indels that are 10 consecutive bases in length or less.

The term “variant motif,” as used herein, refers to any pair of an alternative sequence and a reference sequence that provides a sequence context for a variant, and includes the variant locus and one or more flanking bases at the 5′ end and at the 3′ end of the variant locus. A trinucleotide SNP variant motif, for example, includes a reference sequence XYZ and an alternative sequence XQZ, wherein the change from Y base to Q base is the SNP that is flanked by base X and base Z. In some instances, a variant motif may be longer than a trinucleotide (e.g., an SNP may be flanked by more than one base at one or both of the 5′ and 3′ ends). Thus, in some instances, a variant motif may be 4 or more bases, 5 or more bases, 6 or more bases, 7 or more bases, 8 or more bases, 9 or more bases, 10 or more bases, or 11 or more bases in length.

The term “reference sequence,” as used herein refers to a reference genome or a portion of reference genome (e.g., for a same species as a subject from which a biological sample was taken for analysis). In some embodiments, a reference genome is a reference for any known genome of an organism or virus (e.g., a genome that is partially or completely assembled) that may be used for alignment of sequences from a subject. Example human reference genomes can be accessed from online genome browsers hosted by either the National Center for Biotechnology Information (NCBI) or the University of California, Santa Cruz (UCSC). Example human reference genomes include but are not limited to NCBI build 34 (UCSC equivalent version, hg16), NCBI build 35 (UCSC equivalent version, hg17), NCBI build 36.1 (UCSC equivalent version, hg18), GRCh37 (UCSC equivalent version, hg19), and GRCh38 (UCSC equivalent version, hg38).

As used herein, the term “small nucleotide variant” refers to a sequence variation that occurs when a single nucleotide or when multiple consecutive nucleotides are altered (e.g., in comparison to a reference sequence). An small nucleotide variant may comprise 1, 2, 3, 4, 5, 6, 7, 8, 9, 10, 11, 12, 13, 14, 15, 16, 17, 18, 19, 20, 21, 22, 23, 24, or 25 variant nucleotides. In some cases, an small nucleotide variant may refer to an insertion or deletion (e.g., indel).

As used herein, the term ‘locus’ refers to a physical site or location of a specific nucleotide in a sequence. Thus, the term ‘loci,’ as used herein, refers to more than one physical site or location of multiple nucleotides in a sequence. The locations within a loci may be consecutive or non-consecutive.

The term “homopolymer,” as used herein, generally refers to a polymer or a portion of a polymer comprising identical monomer units, such as a sequence of 0, 1, 2, . . . , N sequential nucleotides. For example, a homopolymer containing sequential A nucleotides may be represented as A, AA, AAA, . . . , up to N sequential A nucleotides. A homopolymer may have a homopolymer sequence. A nucleic acid homopolymer may refer to a polynucleotide or an oligonucleotide comprising consecutive repetitions of a same nucleotide or any nucleotide variants thereof. For example, a homopolymer can be poly(dA), poly(dT), poly(dG), poly(dC), poly(rA), poly(U), poly(rG), or poly(rC). A homopolymer can be of any length. For example, the homopolymer can have a length of at least 2, 3, 4, 5, 10, 20, 30, 40, 50, 100, 200, 300, 400, 500, or more nucleic acid bases. The homopolymer can have from 10 to 500, or 15 to 200, or 20 to 150 nucleic acid bases. The homopolymer can have a length of at most 500, 400, 300, 200, 100, 50, 40, 30, 20, 10, 5, 4, 3, or 2 nucleic acid bases. A molecule, such as a nucleic acid molecule, can include one or more homopolymer portions and one or more non-homopolymer portions. The molecule may be entirely formed of a homopolymer, multiple homopolymers, or a combination of homopolymers and non-homopolymers. In nucleic acid sequencing, multiple nucleotides can be incorporated into a homopolymeric region of a nucleic acid strand. Such nucleotides may be non-terminated to permit incorporation of consecutive nucleotides (e.g., during a single nucleotide flow).

It is understood that aspects and variations of the invention described herein include “consisting of” and/or “consisting essentially of” aspects and variations.

When a range of values is provided, it is to be understood that each intervening value between the upper and lower limit of that range, and any other stated or intervening value in that states range, is encompassed within the scope of the present disclosure. Where the stated range includes upper or lower limits, ranges excluding either of those included limits are also included in the present disclosure.

The section headings used herein are for organization purposes only and are not to be construed as limiting the subject matter described. The description is presented to enable one of ordinary skill in the art to make and use the invention and is provided in the context of a patent application and its requirements. Various modifications to the described instances will be readily apparent to those persons skilled in the art and the generic principles herein may be applied to other embodiments. Thus, the present invention is not intended to be limited to the embodiment shown but is to be accorded the widest scope consistent with the principles and features described herein.

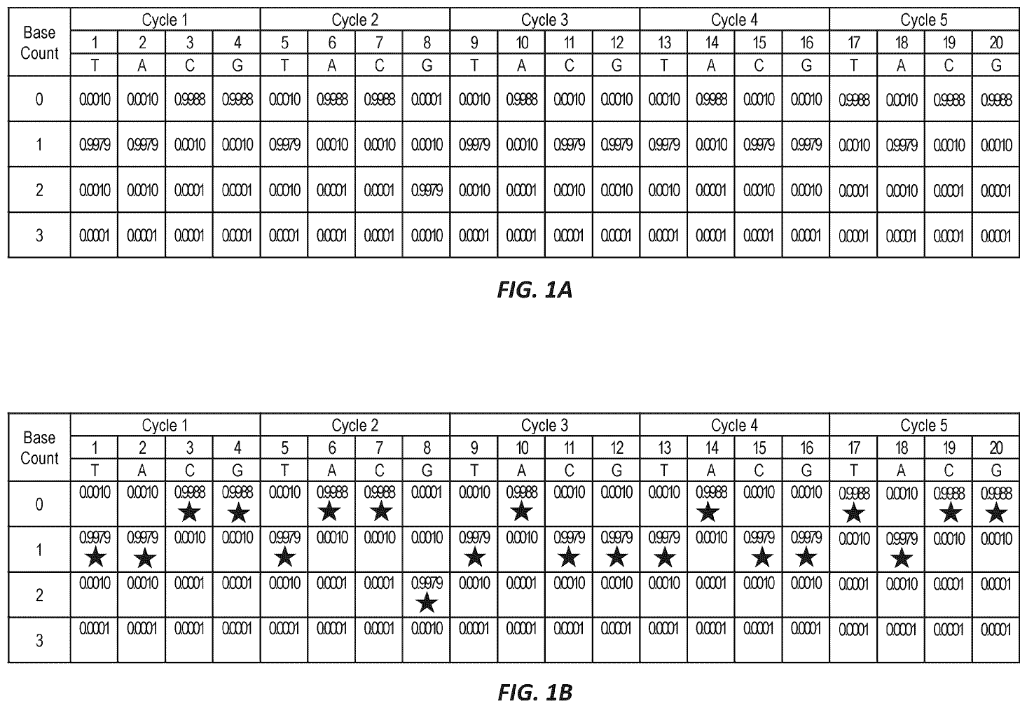

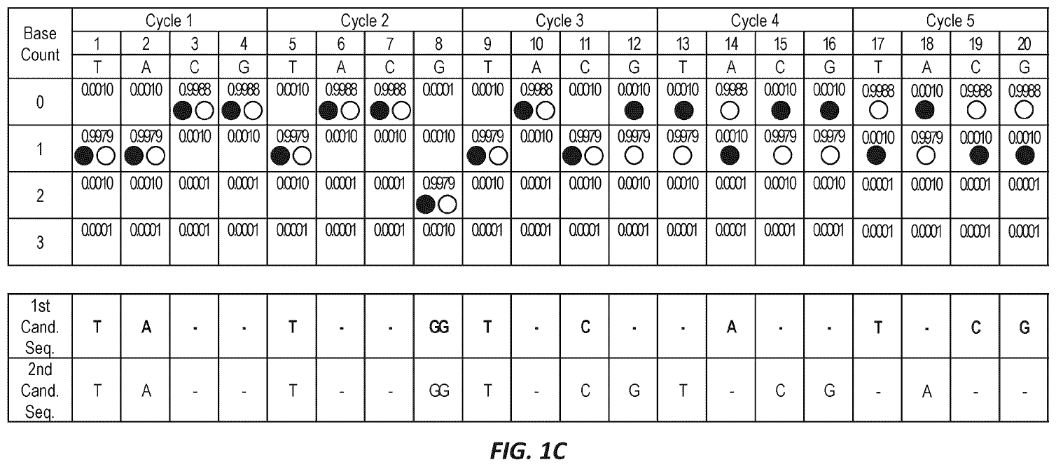

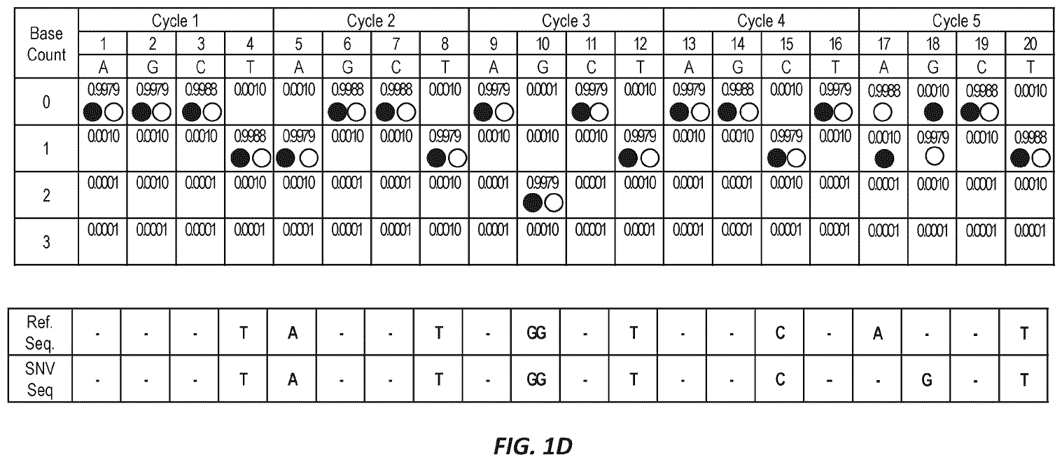

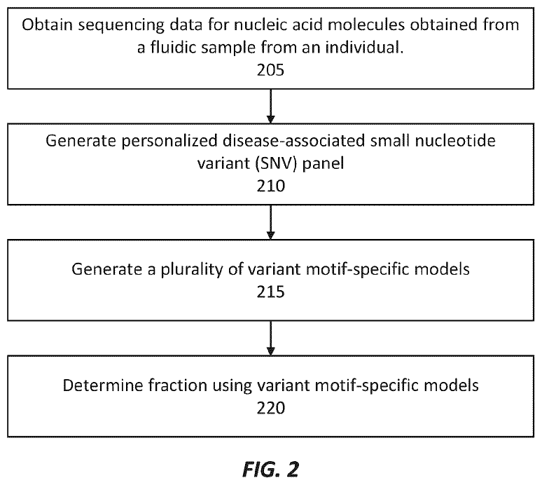

FIGS. 1-2 illustrate processes according to various examples. Any of the process steps may be configured to be performed automatically. These exemplary processes may be performed, for example, using one or more electronic devices implementing a software platform. In some examples, one or more of the exemplary processes are performed using a client-server system, and the blocks of the illustrated processes may be divided up in any manner between the server and a client device. In other examples, the blocks of the exemplary processes are divided up between the server and multiple client devices. Thus, while portions of the exemplary processes are described herein as being performed by particular devices of a client-server system, it will be appreciated that the processes are not so limited. In other examples, one or more of the exemplary processes are performed using only a client device (e.g., user device) or only one or more client devices. In the exemplary processes, some blocks are, optionally, combined, the order of some blocks is, optionally, changed, and some blocks are, optionally, omitted. In some examples, additional steps may be performed in combination with the exemplary processes. Accordingly, the operations as illustrated (and described in greater detail below) are exemplary by nature and, as such, should not be viewed as limiting.

The disclosures of all publications, patents, and patent applications referred to herein are each hereby incorporated by reference in their entireties. To the extent that any reference incorporated by reference conflicts with the instant disclosure, the instant disclosure shall control.

### Personalized Variant Panels

Certain diseases in an individual, such as cancer, can give rise to mutant nucleic acid sequences that provide a signature for the disease. The sequence of the nucleic acid molecules associated with diseased tissue (i.e., a diseased genome) can be compared to the sequence of nucleic acid molecules associated with non-diseased tissue (i.e., a healthy or non-diseased genome) from the same individual. The differences between the diseased genome (or portion thereof) and the non-diseased genome (or portion thereof) determine the variants for the diseased tissue. The personalized diseased-associated small nucleotide variant panel can be in-silico, e.g., not embodied in a set of oligonucleotide primers. The personalized disease-associated small nucleotide variant panel is therefore constructed based on differences between the nucleic acid sequences associated from the diseased tissue and the nucleic acid sequences associated from the healthy (i.e., non-diseased) tissue. In some instances, the sequencing data associated with the diseased tissue and/or healthy tissue is targeted sequencing data. In some instances, the sequencing data associated with the diseased tissue and/or the heathy tissue is untargeted (e.g., genome-wide or whole-genome) sequencing data.

An initial personalized disease-associated small nucleotide variant panel may be generated by detecting variants associated with the disease. For example, nucleic acid molecules from a disease tissue (e.g., a tumor) sample can be sequenced. The tissue may be obtained, for example, by a tissue (e.g., tumor) biopsy. The sample may be a fresh tissue sample or a preserved tissue sample. For example, the diseased tissue may be a formalin-fixed paraffin-embedded (FFPE) tissue sample. Sequencing data for nucleic acid molecules derived from the diseased tissue sample can be used to call disease-associated variants, which can be used to build the personalized disease-associated small nucleotide variant panel.

Some or all of the small nucleotide variants (e.g., single nucleotide polymorphisms (SNPs) or small indels (generally 1-5 bases in length)) detected from the diseased tissue can be used to establish a personalized disease-associated small nucleotide variant panel unique to the disease of that individual. The panel need not include all detected disease-associated small nucleotide variants. For example, the small nucleotide variants may be filtered, for example to exclude small nucleotide variants other than single nucleotide polymorphisms (SNPs). Additionally or alternatively, the initial personalized disease-associated small nucleotide variant panel may be filtered to select small nucleotide variant loci to remove false positives or to select loci with a low false-positive rate.

Minimizing false positive errors (i.e., incorrectly attributing a sequencing read to a nucleic acid molecule derived from diseased tissue) can improve disease fraction detection sensitivity. A subset of small nucleotide variants for which the probability of a false positive read is low can be selected for the personalized disease-associated small nucleotide variant panel by filtering (i.e., excluding) small nucleotide variants at loci with higher false positive rates. The small nucleotide variants can be selected apriori, or can be filtered based on likelihood of supporting the normal/tumor sequence over the alternative.

In some instances, the small nucleotide variant panel is generated by filtering germline variants and/or non-disease (e.g., non-cancer) associated somatic variants from small nucleotide variants associated with the diseased (e.g., cancerous) tissue. Diseased tissue may be sequenced to determine a plurality of variants associated with the disease tissue. The resulting sequencing reads may be compared, for example, to a reference genome, and the variants selected based on the differences between the sequencing reads and the reference genome. The identified variants may include not only variants that are unique to the diseased tissue, but also variants that are found in healthy tissue (for example, variants found in peripheral blood mononuclear cells, e.g., from a buffy coat, or other healthy tissue). For example, variants found in white blood cells can be obtained by sequencing a matching buffy coat sample from the same subject and comparing sequencing data to the reference genome. Although these variants may include cancerous variants, large number of the variants can be caused by age-related clonal hematopoiesis. In some instances, variants identified by buffy coat/white blood cell sequencing are treated as an approximate representative collection of non-cancer related somatic variants. Thus, germline variants (or likely germline) and/or non-disease associated somatic variants (or likely non-diseased related somatic variants) can be characterized by sequencing nucleic acid molecules derived from a healthy tissue sample obtained from the individual and comparing the sequencing reads to the reference genome. The small nucleotide variants associated with the diseased tissue may then be excluded to remove germline variants and/or somatic variants when the disease-associated small nucleotide variant panel is generated.

Any healthy tissue obtained from the individual can be used to determine the sequence of the healthy genome (or portion thereof). The healthy tissue may be, for example, obtained from a fluidic sample (for example, from cell-free nucleic acid molecules (e.g., cfDNA) or healthy blood cells in a fluidic sample), a cheek swab, a biopsy of healthy tissue, or any other suitable method. In some instances, the healthy tissue includes white blood cells, for example peripheral blood mononuclear cells obtained from a buffy coat. In some instances, the healthy tissue includes non-diseased tissue. For example, a tumor biopsy sample (for example, a solid tumor biopsy sample, such as n FFPE tissue sample) may include both healthy (i.e., non-diseased) tissue and diseased tissue. In some instances, the healthy tissue includes a healthy cfDNA sample; for example, an individual may go through routine healthy examination that includes whole genome sequencing (WGS) analysis of a blood sample such as plasma and/or white blood cell containing sample. Such data can be preserved in the individual's health record. When the individual subsequently develops a disease condition such as cancer, the previously obtained sequencing data can be used to establish the healthy baseline for the individual. Conversely, for an individual with a known disease condition (e.g., live cancer or breast cancer) who has undergone treatment (e.g., surgical treatment), a healthy tissue can include one or more taken samples taken right after the treatment when the disease condition can no longer be detected. Such healthy tissue can be used as the baseline sample against which subsequent samples are compared in order to assess if the disease relapses in the individual. A nucleic acid sequencing library can be prepared from the healthy tissue and sequenced to obtain sequencing data attributable to the genome (or portion thereof) of the healthy tissue. Although a small amount of disease tissue may be extracted along with the healthy tissue, the diseased tissue would generally be a minor component that can be ignored for obtaining the sequencing data of the healthy tissue.

The sequence data of the nucleic acid molecules (e.g., genome or portion thereof) associated with the diseased tissue may be determined by obtaining a tissue sample of the diseased tissue, for example a primary or secondary cancer that can be excised, biopsied, or otherwise sampled, and sequencing nucleic acid molecules in the obtained tissue. In some instances, a plurality of samples is obtained from the diseased tissue, which can capture mosaicisms within the diseased tissue (e.g., different clones or sub-clones of the diseased tissue). In some instances, the sequence data associated with the diseased tissue is obtained by sequencing nucleic acid molecules obtained from a fluidic sample (such as from cell-free nucleic acid molecules (e.g., cfDNA) or healthy blood cells in a fluidic sample). A fluidic sample may also include nucleic acid molecules associated with healthy tissue, but the sequencing data associated with the healthy tissue will generally have a substantially higher depth count and can be ignored for the purpose of determining the sequencing data associated with the diseased tissue. The diseased tissue may be sampled, for example, before the start of treatment for the disease (e.g., chemotherapy for the treatment of cancer) or after the start of treatment for the disease.

The personalized disease-associated small nucleotide variant panel includes variants (including loci of the variant and mutational change) of the nucleic acid molecules from diseased tissue compared to the nucleic acid molecules form the non-diseased tissue. The panel may include less than all of the nucleic acid differences between the healthy and diseased tissue, as certain variants may have been undetected due to limits on the sequencing data of the healthy and/or diseased tissue or, arise in regions of the genome that are technically difficult to sequence, e.g. low complexity regions or regions with mapping degeneracies. In some instances, the personalized small nucleotide variant panel includes driver mutations, passenger mutations, or both driver and passenger mutations. In some instances, the small nucleotide variant panel includes mutations in the coding region of the genome, the non-coding region of the genome, or both. The number of variants in the personalized panel depends on the diseased tissue, including the type of diseased tissue, or the severity of the disease. In some instances, the personalized panel includes 2 or more, 5 or more, 10 or more, 25 or more, 50 or more, 100 or more, 200 or more, 300 or more, 500 or more, 1000 or more, 2500 or more, 5000 or more, 10,000 or more, 25,000 or more, 50,000 or more, 100,000 or more, 250,000 or more, 500,000 or more, 1,000,000 or more, 5,000,000 or more loci. In some instances, a variant locus is only included in the personalized small nucleotide variant panel if two or more (e.g., 3 or more, 4 or more, or 5 or more) redundant variant calls are made at any given locus. Screening loci for redundant variant calls limits the number of false positive variants that are introduced into the panel. In some cases, the panel includes only variants that have been verified to be different between diseased and non-diseased tissue by consensus nucleic acid sequencing determined at high confidence.

Not all loci in the initial personalized disease-associated small nucleotide variant panel need to be analyzed for the methods described herein. In some instances, a portion of the loci in the personalized disease-associated small nucleotide variant panel are selected for analysis. Certain loci or variants may be more susceptible to false positive errors than other loci or variants. Additionally, certain sequencing methodologies may be more susceptible to false positive errors than others. In some instances loci are selected from the personalized small nucleotide variant panel based on a false positive error rate at the locus. For example, a locus may be selected if the false positive error rate at that locus is about 1% or less, about 0.5% or less, about 0.25% or less, about 0.1% or less, about 0.05% or less, about 0.025% or less, about 0.01% or less, about 0.005% or less, about 0.0025% or less, or about 0.0001% or less. Solely by way of example, a particular sequencing methodology may have a lower sequencing false positive error rate for detecting a particular mutation (e.g., G to A) mutation than other mutation types (e.g., G to C), and variants with lower false positive error rates may be selected. In some instances, the selected loci include 2 or more, 5 or more, 10 or more, 25 or more, 50 or more, 100 or more, 200 or more, 300 or more, 500 or more, 1000 or more, 2500 or more, 5000 or more, 10,000 or more, 25,000 or more, 50,000 or more, 100,000 or more, 250,000 or more, or 500,000 or more loci. In some instances, all loci in the personalized small nucleotide variant panel are selected.

Filtering germline and non-disease associated somatic variants from the small nucleotide variants associated with diseased tissue is one technique that may be used to select loci from the disease-associated small nucleotide variant panel (or to generate the disease-associated small nucleotide variant panel). CfDNA present in blood can originate from several cell sources, including cancerous and noncancerous cells. Hematopoietic stem cells can include clonal hematopoiesis associated somatic variants, which can lead to the expansion of a clonal population of blood cells. These clonal hematopoiesis associated somatic variants are often non-malignant, and clonal expansion driven by these somatic variants can be referred to as Clonal Hematopoiesis of Indeterminate Potential (CHIP). See, Steensma et al, Clonal hematopoiesis of indeterminate potential and its distinction from myelodysplastic syndromes, Blood, vol., 126, pp. 9-16 (2015). Some studies have shown that least 10% of the elderly population above the age of 70 carry CHIP due to oligoclonal expansion of mutated hematopoietic stem cells. See, Jaiswal et al., Age-Related Clonal Hematopoiesis Associated with Adverse Outcomes, N. Engl. J. Med., vol. 371, no. 26, pp. 2488-2498 (2014). Thus, these non-disease associated somatic variants may be significantly represented in cfDNA even though they are not associated with the disease. See, also, US 2019/0385700 A1, US 2019/0355438 A1, US 2020/0013484 A1, the contents of each of which are incorporated herein by reference for all purposes. Removing these non-disease associated somatic variants from the small nucleotide variant panel can significantly reduce the background error rate. Non-disease associated somatic variants, such as clonal hematopoiesis associate somatic variants, can be identified, for example, by sequencing nucleic acid molecules derived from white blood cells, for example white blood cells in a buffy coat.

In some instances, the small nucleotide variant panel includes small nucleotide variants associated with the diseased tissue that have been filtered to remove germline and non-disease associated somatic variants (i.e., somatic variants unrelated to the disease). For example, these non-disease associated somatic variants can be determined by sequencing nucleic acid molecules derived from healthy tissue (such as a sample containing white blood cells, like a buffy coat). Removing germline and non-disease associated somatic variants detected by sequencing nucleic acid molecules obtained from white blood cells (e.g., from the buffy coat) may be particularly useful when the level of disease is measured by sequencing cfDNA. When the cfDNA is sequenced for analysis, both disease-associated variants arising from the tumor and non-disease associated somatic variants and germline variants are detected. Removing the germline and non-disease associated somatic variants from analysis can reduce erroneous attribution to the ctDNA. Thus, the false positive error rate (that is, small nucleotide variants that are incorrectly attributed to the diseased tissue) can be reduced by removing non-disease associated somatic variants.

In the event that certain small nucleotide variant loci are not sequenced when nucleic acid molecules derived from the non-diseased tissue are sequenced, small nucleotide variants associated with these loci can be excluded from the personalized disease-associated small nucleotide variant panel. This helps to minimize the risk that an small nucleotide variant in the panel is not incidentally a germline variant or non-disease associated somatic variant that simply evaded detection when sequencing the nucleic acid molecules from the non-diseased tissue. That is, nucleic acid molecules derived from a non-diseased tissue sample obtained from the individual are sequenced to obtain non-diseased tissue sequencing data, and small nucleotide variants at loci that have no sequencing coverage within the non-diseased tissue sequencing data can be excluded from the personalized disease-associate small nucleotide variant panel, small nucleotide variants at loci that have no sequencing coverage within the non-diseased tissue sequencing data.

The small nucleotide variants in the disease-associated small nucleotide variant panel may be selected by (or the disease-associated small nucleotide variant panel may be generated by) excluding common variant alleles, for example, variants with a frequency greater than a predetermined frequency threshold from a general population. Common variants are likely germline mutations and not unique to the diseased tissue, and therefore can be excluded to reduce errors. In some instances, the predetermined frequency threshold is about 0.005 (or more), about 0.01 or more, about 0.02 or more, or about 0.05 or more. Thus, the false positive error rate (that is, small nucleotide variants that are incorrectly attributed to the diseased tissue) can be reduced by removing SNVs that are common to the general population, and thus likely attributable to germline variance.

Optionally, small nucleotide variants at loci with two or more non-reference alleles may be excluded from the personalized disease-associated small nucleotide variant panel. That is, generally small nucleotide variants have a reference allele and a variant allele. However, an small nucleotide variant that has two or more variant alleles may be excluded to ensure variant signal may be attributable to a variant associated with the disease, instead of background noise. Such small nucleotide variants are relatively rare, so excluding multivariate small nucleotide variants does not greatly reduce the amount of data analyzed for a subject.

small nucleotide variants within low complexity regions (LCRs) may optionally be excluded from the personalized disease-associated small nucleotide variant panel. Low complexity regions are generally known in the art, and can include, for example, a homopolymer region, a region with one or more short tandem repeat sequences, a region with one or more variable tandem repeat sequences. A low complexity region may be identified using a low complexity filter (e.g., such as Dust, SEG, or mdust). See e.g., Li, Toward better understanding of artifacts in variant calling from high-coverage samples, Bioinformatics, vol. 30, no. 20, pp. 2843-2851 (2014) and Ye et al., BLAST. improvements for better sequence analysis, Nucleic Acids Research, vol. 34, pp. W6-W9 (2006). In some instances, the method includes excluding from the personalized disease-associated small nucleotide variant panel at least one small nucleotide variant within a homopolymer region. In some instances, the method includes excluding from the personalized disease-associated small nucleotide variant panel at least one small nucleotide variant within a short tandem repeat or within a variable tandem repeat.

In some instances, the mapping quality of an alternate read may be significantly lower than the mapping quality of a non-variant (i.e., reference) read to a reference sequence. small nucleotide variants at loci associated with low mapping quality of reads may be excluded from the small nucleotide variant panel, which can limit bias of the variant signal. For example, sequencing reads obtained by sequencing the diseased tissue (which includes alternate reads and reference reads) can be mapped to a reference sequence, and a mapping quality score can be determined for each read. The sequencing read may be mapped to the reference sequence, for example using a Burrows-Wheeler Alignment (BWA) algorithm or other suitable alignment algorithm. small nucleotide variants at loci associated with a predetermined number or proportion of sequencing reads (e.g., at least about 75%, at least about 80%, at least about 85%, at least about 90%, at least about 95%, or at least about 99%) that have a Phred mapping quality score below a predetermined mapping quality threshold may be excluded from personalized disease-associated small nucleotide variant panel. The sequencing reads can be obtained by sequencing nucleic acid molecules from the diseased tissue, or the sequencing reads can be obtained by sequencing cfDNA molecules. The predetermined mapping quality threshold may be set depending on a desired error tolerance. For example, a Phred mapping quality score of 60 using the Phred scale is equivalent to a 10⁻⁶ error probability or less. In some instances, the predetermined mapping quality threshold is a Phred mapping quality score of about 40 or higher, about 50 or higher, about 60 or higher, about 70 or higher, or about 80 or higher.

Some small nucleotide variant loci in in the initial personalized disease-associated small nucleotide variant panel have a bias for reference reads or for alternate reads. Reference and alternative alleles (or more precisely haplotypes) are from the output of a variant calling algorithm that yielded our original list of variants. In rare cases, this algorithm does not determine the alternative allele properly, causing all the reads that actually match it to have a low likelihood. These small nucleotide variants may be excluded from the personalized disease-associated small nucleotide variant panel. The bias can be determined by sequencing nucleic acid molecules derived from the diseased tissue.

In some instances, the small nucleotide variants in the disease-associated small nucleotide variant panel may be selected by (or the disease-associated small nucleotide variant panel may be generated by) excluding variants detected in the nucleic acid sequencing data having an allele frequency greater than a predetermined threshold or greater than a statistical threshold. cfDNA derived from a diseased tissue is generally the minor fraction of the cfDNA, and variants having a high allele frequency are likely attributable to germline and/or somatic variants unrelated to the disease (e.g., non-disease associate somatic variants or somatic variants relating to a different condition or disease), and may be excluded from analysis for measuring the level of disease. Plotting a histogram of allele frequency will generally provide a lower cluster of allele frequency, which is generally attributable to the diseased tissue or sequencing noise, and a higher cluster of allele frequency, which is generally attributable to germline and/or somatic variants. In some instances, a statistical parameter is determined to distinguish the lower cluster of allele frequency and the higher cluster of allele frequency, and variants associated with the higher cluster of allele frequency can be excluded. In some instances, the predetermined threshold is used to exclude the variants in the higher cluster of allele frequency. The predetermined threshold may be, for example, about 0.2 or higher, about 0.25 or higher, or about 0.3, or higher.

Small nucleotide variants associated with low allele fraction or high allele fraction variant calls when sequencing the diseased tissue may be excluded from the personalized disease-associated small nucleotide variant panel. Variants with an allele fraction in the nucleic acid molecules derived from the diseased tissue sample lower than a predetermined low-fraction threshold (e.g., less than 10%, less than 7%, or less than 5%) may be excluded from the small nucleotide variant panel. Such low allele fraction variants may be due to variant calling artifacts or rare (or sub-clonal) mutations, and can intrude noise and/or bias. Similarly, variants with an allele fraction in the nucleic acid molecules derived from the diseased tissue sample higher than a predetermined high-fraction threshold (e.g., more than 50%, more than 55%, or more than 60%) may be excluded from the small nucleotide variant panel. Such high allele fraction variants may be due to germline variants or copy number variants, and can intrude noise and/or bias.

Selection of small nucleotide variants for the personalized disease-associated small nucleotide variant panel may include excluding small nucleotide variants that result in outlier loci-specific fractions. For example, sequence reads from the sequencing data can be characterized as an alternative read or a reference read. For a given small nucleotide variant locus, a locus-specific fraction F can be determined, for locus i, according to:

$$F = \frac{{N}_{i}^{alt}}{{N}_{i}^{alt} + {N}_{i}^{ref}}$$

wherein Nᵢ^{alt} is the number of alternative sequencing reads at locus i, and Nᵢ^{ref} is the number of reference sequencing reads at locus i. The likelihood of the measured fraction at any given locus, and small nucleotide variants having a locus associated with a likelihood below a predetermined threshold (i.e., outlier small nucleotide variants) can be excluded from the personalized disease-associated small nucleotide variant panel. Once the small nucleotide variant has been excluded, the process of identifying and excluding one or more outlier small nucleotide variants may be performed iteratively.

Other techniques may be used in addition or in the alternative to select small nucleotide variants for the disease-associated small nucleotide variant panel or to generate the disease-associated small nucleotide variant panel. For example, in some instances, small nucleotide variants may be included in the disease-associated small nucleotide variant panel (or the disease-associated small nucleotide variant panel may be generated to include small nucleotide variants) only when the disease-associated variant is supported by two or more (e.g., 3, 4, 5, or more) sequencing reads obtained when sequencing the nucleic acid molecules derived from the diseased tissue. By requiring two or more sequencing reads to support the variant associated with the diseased tissue, the likelihood of false positives can be reduced (for example, by limiting the number of variants called by sequencing or other errors when analyzing the diseased tissue). Thus, the false positive error rate (that is, small nucleotide variants that are incorrectly attributed to the diseased tissue) can be reduced by removing small nucleotide variants that are not robustly supported by the sequencing data obtained by sequencing nucleic acid molecules derived from the diseased tissue.

In some instances, the small nucleotide variants in the disease-associated small nucleotide variant panel may be selected by (or the disease-associated small nucleotide variant panel may be generated by) excluding variants in a homopolymer region (a stretch of consecutive nucleotides having the same baes type). In some instances, the homopolymer region contains 3, 4, 5, 6, 7, 8, 9, 10, or more continuous nucleotides having the same base type. Variants in homopolymer regions are susceptible to being false positive variants, and may not accurately reflect the diseased tissue. Thus, the false positive error rate (that is, small nucleotide variants that are incorrectly attributed to the diseased tissue) can be reduced by removing small nucleotide variants that fall within homopolymer regions.

In some instances, the small nucleotide variants in the disease-associated small nucleotide variant panel may be selected by (or the disease-associated small nucleotide variant panel may be generated by) excluding variants not supported by complementary strands among nucleic acid molecules derived from the disease tissue. For example, if the variant is called in a sequencing read associated with a first strand but a complementary variant is not called in a second strand complementary to the first strand, then a sequencing error or other artifact may be assumed, and the variant can be excluded from further analysis. Thus, the false positive error rate (that is, small nucleotide variants that are incorrectly attributed to the diseased tissue) can be reduced by removing small nucleotide variants that are not robustly supported by the sequencing data obtained by sequencing nucleic acid molecules derived from the diseased tissue.

In some instances, the small nucleotide variants in the disease-associated small nucleotide variant panel may be selected by (or the disease-associated small nucleotide variant panel may be generated by) including variants that induce a cycle shift (e.g., a flowgram signal shifts by one or more flow cycles relative to the reference based on a flow cycle order) and/or generate a new zero or new non-zero signal in sequencing data. See, for example, U.S. patent application Ser. No. 16/864,981 (corresponding U.S. Pat. Pub. 2020/0372971 A1) and published International application WO 2020/227137, the contents of each of which are incorporated herein by reference in their entirety for all purposes. Because a cycle shift event is unlikely in the absence of a true positive event (as further explained herein), in some instances, loci from the disease-associated small nucleotide variant panel may be selected if variants at the loci result in a cycle shift event. Thus, the false positive error rate (that is, small nucleotide variants that are incorrectly attributed to the diseased tissue) can be reduced by including only small nucleotide variants that provide a strong signal.

By way of example, small nucleotide variants in the personalized disease-associated small nucleotide variant panel may be associated with small nucleotide variant sequencing data that differs from reference sequencing data associated with a reference sequence at two or more flow positions when the small nucleotide variant sequencing data and the reference sequencing data are sequenced using non-terminating nucleotides provided in separate nucleotide flows according to a flow-cycle order comprising a plurality of flow positions, wherein the flow positions correspond to nucleotide flows. In some instances, at least 90%, at least 95%, at least 99%, or all of the small nucleotide variants in the personalized disease-associated small nucleotide variant panel may be associated with small nucleotide variant sequencing data that differs from reference sequencing data associated with a reference sequence at two or more flow positions when the small nucleotide variant sequencing data and the reference sequencing data are sequenced using non-terminating nucleotides provided in separate nucleotide flows according to a flow-cycle order comprising a plurality of flow positions, wherein the flow positions correspond to nucleotide flows.

In some instances, small nucleotide variants in the personalized disease-associated small nucleotide variant panel are associated with small nucleotide variant sequencing data that differs from reference sequencing data associated with a reference sequence across one or more flow cycles when the sequencing data and the reference sequencing data are sequenced using non-terminating nucleotides provided in separate nucleotide flows according to the flow-cycle order. In some instances, at least 90%, at least 95%, at least 99%, or all of the small nucleotide variants in the personalized disease-associated small nucleotide variant panel are associated with small nucleotide variant sequencing data that differs from reference sequencing data associated with a reference sequence across one or more flow cycles when the sequencing data and the reference sequencing data are sequenced using non-terminating nucleotides provided in separate nucleotide flows according to the flow-cycle order.

The methods described herein can be used to simultaneously analyze different clones or different sub-clones of diseased tissue in the same individual. Different clones of diseased tissue (for example, independent cancer clones) generally have unique or nearly unique variant signatures. Sub-clones of diseased tissue may have some overlapping variants, although generally have a sufficient number of unique variants to select a unique or nearly unique subset of variants. In some instances, sequenced loci are selected from the logical union of variant loci associated with several disease sub-clones and the analysis detects the fraction of sample comprising all disease sub-clones and also detects the fraction of disease from each sub-clone. In some instances, sequenced loci selected for analysis for a given clone or sub-clone are selected to avoid variant overlap (that is, any variant shared by two or more clones or sub-clones is not selected). Thus, the level of disease of the separate clones or sub-clones, or the fraction of nucleic acid molecules associated with the separate clones or sub-clones, can be determined using the same sample from the individual. In some instances, one or more of the clones or sub-clones is refractory to one or more cancer treatments, and the method can be used to monitor progression or regression of the refractor clone or sub-clone.

### Patient Samples and Sequencing

Fluidic samples are a relatively non-invasive method for obtaining a sample from an individual. Such fluidic samples can include, for example, a blood, plasma, saliva, fecal, or urine sample. Additionally, for residual, malignant, or other disease with no (or no significant) primary or solid diseased tissue, the fluidic sample allows one to obtain (e.g., allows the collection of) nucleic acid molecules associated with the diseased tissue without a tumor biopsy. The methods described herein are therefore particularly useful when the location of the diseased tissue is unknown or when the solid diseased tissue is too small to sample.

The fluidic sample taken from an individual with a disease, such as cancer, generally has cell-free DNA (or “cfDNA”), which includes nucleic acid molecules derived from the cancer tissue and nucleic acid molecules derived from the non-diseased tissue. The nucleic acid samples from which the sequencing data is obtained may be, but need not be, cfDNA. For example, a fluidic sample can provide other nucleic acids from which the sequencing data can be obtained. For example, if the disease is a blood disease (e.g., a hematological cancer), blood cells can be obtained from a blood sample, and the nucleic acid molecules from the blood cells can be sequenced to obtain the sequencing data. In some instances, the nucleic acid molecules are cell-free RNA molecules obtained from the fluidic sample.

Nucleic acid molecules may be sequenced using any suitable sequencing method to obtain sequencing data from the nucleic acid molecules. Exemplary sequencing methods can include, but are not limited to, high-throughput sequencing, next-generation sequencing, sequencing-by-synthesis, flow sequencing, massively-parallel sequencing, shotgun sequencing, single-molecule sequencing, nanopore sequencing, pyrosequencing, semiconductor sequencing, sequencing-by-ligation, sequencing-by-hybridization, RNA-Seq, digital gene expression, single molecule sequencing by synthesis (SMSS), clonal single molecule array, sequencing by ligation, and Maxim-Gilbert sequencing. In some instances, the nucleic acid molecules may be sequenced using a high-throughput sequencer, such as an Illumina HiSeq2500, Illumina HiSeq3000, Illumina HiSeq4000, Illumina HiSeqX, Roche 454, Life Technologies Ion Proton, or open sequencing platform as described in U.S. Pat. No. 10,267,790, which is incorporated herein by reference in its entirety. Other methods of sequencing and sequencing systems are known in the art. In some instances, the nucleic acid molecules are sequenced using a sequencing-by-synthesis (SBS) method. In some instances, the nucleic acid molecules are sequenced using a “natural sequencing-by-synthesis” or “non-terminated sequencing-by-synthesis” method (see U.S. Pat. No. 8,772,473, which is incorporated herein by reference in its entirety).

The selected sequencing method can impact the false positive error rate, either uniformly or as applied to specific variant types. As discussed above, in some instances, the loci selected for analysis from the personalized small nucleotide variant panel can be selected based on the false positive error rate for a given variant. In some instances, the nucleic acid molecules are sequenced using two or more different sequencing methods. By using two or more different sequencing methods that have different false positive error rates for different variants, a larger number of variants may be selected, with the false positive error rate applied to the different sequencing method. For example, certain sequencing methods rely on a predetermined nucleotide sequencing cycle (e.g., CTAG, ATCG, TCAG, etc.), and the sequencing error rate of a variant type can depend on the order of the cycle. Accordingly, in some instances, the sequencing data is obtained by sequencing nucleic acid molecules according to a first predetermined nucleotide sequencing cycle, and re-sequencing the nucleic acid molecules according to a different predetermined nucleotide sequencing cycle order. In some instances, the sequencing data is obtained using two, three, four or more different nucleotide sequencing cycle orders.

In some instances, the sequencing data is untargeted. Certain sequencing methodologies rely on targeting specific regions or loci of the genome to limit the breadth of sequencing and/or enrich specific regions. Common methods of targeting include hybridization targeting (for example using a nucleic acid probe attached to a label or bead is used to selectively target regions of the nucleic acid molecules in a sample for targeted sequencing), primer-based targeting (for example, using nucleic acid primers to amplify targeted nucleic acid regions through amplification (e.g., PCR)), array-based capture, and in-solution capture methods. The targeted regions may be, for example, previously identified variants, genes in the genome that are known drivers of cancer proliferation, or mutational hotspots within the genome. However, targeted sequencing ignores significant portions of information throughout the diseased tissue genome that can be used by the methods described herein.

The method is optionally performed using sequencing data obtained through whole genome sequencing (WGS). By utilizing whole genome sequencing, a larger number of variant loci can be detected and used for analysis. The detected signal increases at a greater rate than the noise with an increasing number of analyzed loci, and by utilizing the full genome a larger amount of data can be analyzed with a less complex preparation. Thus, in some instances, no region of the genome is targeted. In some instances the sequencing data is obtained from untargeted whole-genome sequencing.

Because the methods descried herein can be used with a large breadth of sequencing data (for example, untargeted or whole-genome sequencing data), the average sequencing depth need not be as high as targeted enrichment methods. For example, in some instances, the average sequencing depth of the sequencing data is about 100 or less, about 50 or less, about 25 or less, about 10 or less, about 5 or less, about 1 or less, about 0.5 or less, about 0.25 or less, about 0.1 or less, about 0.05 or less, about 0.025 or less, or about 0.01 or less. In some instances, the average sequencing depth is about 0.01 to about 1000, or any depth therebetween.

In some instances, the sequencing data is obtained without amplifying the nucleic acid molecules prior to establishing sequencing colonies (also referred to as sequencing clusters). Methods for generating sequencing colonies include bridge amplification or emulsion PCR. Methods that rely on shotgun sequencing and calling a consensus sequence generally label nucleic acid molecules using unique molecular identifiers (UMIs) and amplify the nucleic acid molecules to generate numerous copies of the same nucleic acid molecules that are independently sequenced. The amplified nucleic acid molecules can then be attached to a surface and bridge amplified to generate sequencing clusters that are independently sequenced. The UMIs can then be used to associate the independently sequenced nucleic acid molecules. However, the amplification process can introduce errors into the nucleic acid molecules, for example due to the limited fidelity of the DNA polymerase. As discussed above, the presently provided methods can be performed without calling a consensus sequence, and therefore this initial amplification process is not needed and can be avoided to reduce the false positive error rate. In some instances, the nucleic acid molecules are not amplified prior to amplification to generate colonies for obtaining sequencing data. In some instances, the nucleic acid sequencing data is obtained without the use of unique molecular identifiers (UMIs).

The proportion of an individual sample in a pool of samples can be determined using the pooled sequencing data and the sequencing data associated with the individual. The genome of the individual has a unique variant signature, which can be used to determine the proportion of nucleic acid molecules that are attributable to that individual. Thus, samples from a plurality of individuals can be pooled and the portion of nucleic acid molecules in the pooled sample associated with the individual can be determined without the use of sample identification barcodes.

In some instances, the individual has a disease or previously had a disease. In some instances, the disease is cancer. Exemplary cancers that are encompassed by the methods described herein include, but are not limited to, acute lymphoblastic leukemia, acute myeloid leukemia, adenocarcinoma (for example, prostate, small intestine, endometrium, cervical canal, large intestine, lung, pancreas, gullet, intestinum rectum, uterus, stomach, mammary gland, and ovary), B-cell lymphoma, breast cancer, carcinoma, cervical cancer, chronic myelogenous leukemia, colon cancer, esophageal cancer, glioblastoma, glioma, a hematological cancer, Hodgkin's lymphoma, leukemia, lymphoma, lung cancer (e.g., non-small cell lung cancer), liver cancer, melanoma (e.g., metastatic malignant melanoma), multiple myeloma, a neoplastic malignancy, neuroblastoma, non-Hodgkin's lymphoma, ovarian cancer, pancreatic adenocarcinoma, prostate cancer (e.g., hormone refractory prostate adenocarcinoma), renal cancer (e.g., clear cell carcinoma), squamous carcinoma (for example, cervical canal, eyelid, tunica conjunctiva, vagina, lung, oral cavity, skin, urinary bladder, tongue, larynx, and gullet), squamous cell carcinoma of the head and neck, T-cell lymphoma, and thyroid cancer. In some instances, the cancer is refractory to one or more treatments. In some instances, the cancer is in remission or suspected of being in remission.

### Flow Sequencing Methods and Cycle Shift Detection

Exemplary methods of sequencing nucleic acid molecules can include sequencing the nucleic acid molecules using a flow sequencing method to generate the sequencing data. Flow sequencing methods can allow for high confidence selection of variant loci in the disease-associated small nucleotide variant panel, for example by selecting loci or variants with low error rates. For example, in some instances, the loci in the disease-associated small nucleotide variant panel may be selected by (or the disease-associated small nucleotide variant panel may be generated by) including only those variants that induce a cycle shift (i.e., the flowgram signal shifts by one full cycle (e.g., 4 flow positions) relative to the reference based on a flow cycle order) and/or generate a new zero or new non-zero signal in sequencing data, as further described herein.

Flow sequencing methods can include extending a primer bound to a template polynucleotide molecule according to a pre-determined flow cycle where, in any given flow position, a single type of nucleotide is accessible to the extending primer. In some instances, at least some of the nucleotides of the particular type include a label, which upon incorporation of the labeled nucleotides into the extending primer renders a detectable signal. The resulting sequence by which such nucleotides are incorporated into the extended primer should be the reverse complement of the sequence of the template polynucleotide molecule. In some instances, for example, sequencing data is generated using a flow sequencing method that includes extending a primer using labeled nucleotides, and detecting the presence or absence of a labeled nucleotide incorporated into the extending primer. Flow sequencing methods may also be referred to as “natural sequencing-by-synthesis,” or “non-terminated sequencing-by-synthesis” methods. Exemplary methods are described in U.S. Pat. No. 8,772,473, which is incorporated herein by reference in its entirety. While the following description is provided in reference to flow sequencing methods, it is understood that other sequencing methods may be used to sequence all or a portion of the sequenced region. For example, the sequencing data discussed herein can be generated using pyrosequencing methods.

Flow sequencing includes the use of nucleotides to extend the primer hybridized to the polynucleotide. Nucleotides of a given base type (e.g., A, C, G, T, U, etc.) can be mixed with hybridized templates to extend the primer if a complementary base is present in the template strand. The nucleotides may be, for example, non-terminating nucleotides. When the nucleotides are non-terminating, more than one consecutive base can be incorporated into the extending primer strand if more than one consecutive complementary base is present in the template strand. The non-terminating nucleotides contrast with nucleotides having 3′ reversible terminators, wherein a blocking group is generally removed before a successive nucleotide is attached. If no complementary base is present in the template strand, primer extension ceases until a nucleotide that is complementary to the next base in the template strand is introduced. At least a portion of the nucleotides can be labeled so that incorporation can be detected. Most commonly, only a single nucleotide type is introduced at a time (i.e., discretely added), although two or three different types of nucleotides may be simultaneously introduced in certain embodiments. This methodology can be contrasted with sequencing methods that use a reversible terminator, wherein primer extension is stopped after extension of every single base before the terminator is reversed to allow incorporation of the next succeeding base.

The nucleotides can be introduced at a flow order during the course of primer extension, which may be further divided into flow cycles. The flow cycles are a repeated order of nucleotide flows, and may be of any length. Nucleotides are added stepwise, which allows incorporation of the added nucleotide to the end of the sequencing primer of a complementary base in the template strand is present. Solely by way of example, the flow order of a flow cycle may be A-T-G-C, or the flow cycle order may be A-T-C-G. Alternative orders may be readily contemplated by one skilled in the art. The flow cycle order may be of any length, although flow cycles containing four unique base type (A, T, C, and G in any order) are most common. In some instances, the flow cycle includes 5, 6, 7, 8, 9, 10, 11, 12, 13, 14, 15, 16, 17, 18, 19, 20 or more separate nucleotide flows in the flow cycle order. Solely by way of example, the flow cycle order may be T-C-A-C-G-A-T-G-C-A-T-G-C-T-A-G, with these 16 separately provided nucleotides provided in this flow-cycle order for several cycles. Between the introductions of different nucleotides, unincorporated nucleotides may be removed, for example by washing the sequencing platform with a wash fluid.

A polymerase can be used to extend a sequencing primer by incorporating one or more nucleotides at the end of the primer in a template-dependent manner. In some instances, the polymerase is a DNA polymerase. The polymerase may be a naturally occurring polymerase or a synthetic (e.g., mutant) polymerase. The polymerase can be added at an initial step of primer extension, although supplemental polymerase may optionally be added during sequencing, for example with the stepwise addition of nucleotides or after a number of flow cycles. Exemplary polymerases include a DNA polymerase, an RNA polymerase, a thermostable polymerase, a wild-type polymerase, a modified polymerase, Bst DNA polymerase, Bst 2.0 DNA polymerase Bst 3.0 DNA polymerase, Bsu DNA polymerase, E. coli DNA polymerase I, T7 DNA polymerase, bacteriophage T4 DNA polymerase Φ29 (phi29) DNA polymerase, Taq polymerase, Tth polymerase, Tli polymerase, Pfu polymerase, and SeqAmp DNA polymerase.

The introduced nucleotides can include labeled nucleotides when determining the sequence of the template strand, and the presence or absence of an incorporated labeled nucleic acid can be detected to determine a sequence. The label may be, for example, an optically active label (e.g., a fluorescent label) or a radioactive label, and a signal emitted by or altered by the label can be detected using a detector. The presence or absence of a labeled nucleotide incorporated into a primer hybridized to a template polynucleotide can be detected, which allows for the determination of the sequence (for example, by generating a flowgram). In some instances, the labeled nucleotides are labeled with a fluorescent, luminescent, or other light-emitting moiety. In some instances, the label is attached to the nucleotide via a linker. In some instances, the linker is cleavable, e.g., through a photochemical or chemical cleavage reaction. For example, the label may be cleaved after detection and before incorporation of the successive nucleotide(s). In some instances, the label (or linker) is attached to the nucleotide base, or to another site on the nucleotide that does not interfere with elongation of the nascent strand of DNA. In some instances, the linker comprises a disulfide or PEG-containing moiety.

In some instances, the nucleotides introduced include only unlabeled nucleotides, and in some instances the nucleotides include a mixture of labeled and unlabeled nucleotides. For example, in some instances, the portion of labeled nucleotides compared to total nucleotides is about 90% or less, about 80% or less, about 70% or less, about 60% or less, about 50% or less, about 40% or less, about 30% or less, about 20% or less, about 10% or less, about 5% or less, about 4% or less, about 3% or less, about 2.5% or less, about 2% or less, about 1.5% or less, about 1% or less, about 0.5% or less, about 0.25% or less, about 0.1% or less, about 0.05% or less, about 0.025% or less, or about 0.01% or less. In some instances, the portion of labeled nucleotides compared to total nucleotides is about 100%, about 95% or more, about 90% or more, about 80% or more about 70% or more, about 60% or more, about 50% or more, about 40% or more, about 30% or more, about 20% or more, about 10% or more, about 5% or more, about 4% or more, about 3% or more, about 2.5% or more, about 2% or more, about 1.5% or more, about 1% or more, about 0.5% or more, about 0.25% or more, about 0.1% or more, about 0.05% or more, about 0.025% or more, or about 0.01% or more. In some instances, the portion of labeled nucleotides compared to total nucleotides is about 0.01% to about 100%, such as about 0.01% to about 0.025%, about 0.025% to about 0.05%, about 0.05% to about 0.1%, about 0.1% to about 0.25%, about 0.25% to about 0.5%, about 0.5% to about 1%, about 1% to about 1.5%, about 1.5% to about 2%, about 2% to about 2.5%, about 2.5% to about 3%, about 3% to about 4%, about 4% to about 5%, about 5% to about 10%, about 10% to about 20%, about 20% to about 30%, about 30% to about 40%, about 40% to about 50%, about 50% to about 60%, about 60% to about 70%, about 70% to about 80%, about 80% to about 90%, about 90% to less than 100%, or about 90% to about 100%.

Prior to generating the sequencing data, the polynucleotide is hybridized to a sequencing primer to generate a hybridized template. The polynucleotide may be ligated to an adapter during sequencing library preparation. The adapter can include a hybridization sequence that hybridizes to the sequencing primer. For example, the hybridization sequence of the adapter may be a uniform sequence across a plurality of different polynucleotides, and the sequencing primer may be a uniform sequencing primer. This allows for multiplexed sequencing of different polynucleotides in a sequencing library.

The polynucleotide may be attached to a surface (such as a solid support) for sequencing. The polynucleotides may be amplified (for example, by bridge amplification or other amplification techniques) to generate polynucleotide sequencing colonies. The amplified polynucleotides within the cluster are substantially identical or complementary (some errors may be introduced during the amplification process such that a portion of the polynucleotides may not necessarily be identical to the original polynucleotide). Colony formation allows for signal amplification so that the detector can accurately detect incorporation of labeled nucleotides for each colony. In some cases, the colony is formed on a bead using emulsion PCR and the beads are distributed over a sequencing surface. Examples for systems and methods for sequencing can be found in U.S. patent Ser. No. 10,344,328, which is incorporated herein by reference in its entirety.

The primer hybridized to the polynucleotide is extended through the nucleic acid molecule using the separate nucleotide flows according to the flow order (which may be cyclical according to a flow-cycle order), and incorporation of a nucleotide can be detected as described above, thereby generating the sequencing data set for the nucleic acid molecule.

Primer extension using flow sequencing allows for long-range sequencing on the order of hundreds or even thousands of bases in length. The number of flow steps or cycles can be increased or decreased to obtain the desired sequencing length. Extension of the primer can include one or more flow steps for stepwise extension of the primer using nucleotides having one or more different base types. In some instances, extension of the primer includes between 1 and about 1000 flow steps, such as between 1 and about 10 flow steps, between about 10 and about 20 flow steps, between about 20 and about 50 flow steps, between about 50 and about 100 flow steps, between about 100 and about 250 flow steps, between about 250 and about 500 flow steps, or between about 500 and about 1000 flow steps. The flow steps may be segmented into identical or different flow cycles. The number of bases incorporated into the primer depends on the sequence of the sequenced region, and the flow order used to extend the primer. In some instances, the sequenced region is about 1 base to about 4000 bases in length, such as about 1 base to about 10 bases in length, about 10 bases to about 20 bases in length, about 20 bases to about 50 bases in length, about 50 bases to about 100 bases in length, about 100 bases to about 250 bases in length, about 250 bases to about 500 bases in length, about 500 bases to about 1000 bases in length, about 1000 bases to about 2000 bases in length, or about 2000 bases to about 4000 bases in length.

Sequencing data can be generated based on the detection of an incorporated nucleotide and the order of nucleotide introduction. Take, for example, the flowing extended sequences (i.e., each reverse complement of a corresponding template sequence): CTG, CAG, CCG, CGT, and CAT (assuming no preceding sequence or subsequent sequence subjected to the sequencing method), and a repeating flow cycle of T-A-C-G (that is, sequential addition of T, A, C, and G nucleotides in repeating cycles). A particular type of nucleotides at a given flow position would be incorporated into the primer only if a complementary base is present in the template polynucleotide. An exemplary resulting flowgram is shown in Table 1, where 1 indicates incorporation of an introduced nucleotide and 0 indicates no incorporation of an introduced nucleotide. The flowgram can be used to derive the sequence of the template strand. For example, the sequencing data (e.g., flowgram) discussed herein represent the sequence of the extended primer strand, and the reverse complement of which can readily be determined to represent the sequence of the template strand. An asterisk (*) in Table 1 indicates that a signal may be present in the sequencing data if additional nucleotides are incorporated in the extended sequencing strand (e.g., a longer template strand).

TABLE 1

| | Cycle 1 | | | | Cycle 2 | | | | Cycle 3 | | | |
|---|---|---|---|---|---|---|---|---|---|---|---|---|
| Flow Position | 1 | 2 | 3 | 4 | 5 | 6 | 7 | 8 | 9 | 10 | 11 | 12 |
| Base in Flow | T | A | C | G | T | A | C | G | T | A | C | G |
| Extended sequence: CTG | 0 | 0 | 1 | 0 | 1 | 0 | 0 | 1 | * | * | * | * |
| Extended sequence: CAG | 0 | 0 | 1 | 0 | 0 | 1 | 0 | 1 | * | * | * | * |
| Extended sequence: CCG | 0 | 0 | 2 | 1 | * | * | * | * | * | * | * | * |
| Extended sequence: CGT | 0 | 0 | 1 | 1 | 1 | * | * | * | * | * | * | * |
| Extended sequence: CAT | 0 | 0 | 1 | 0 | 0 | 1 | 0 | 0 | 1 | * | * | * |

The flowgram may be binary or non-binary. A binary flowgram detects the presence (1) or absence (0) of an incorporated nucleotide. A non-binary flowgram can more quantitatively determine a number of incorporated nucleotides from each stepwise introduction. For example, an extended sequence of CCG would include incorporation of two C bases in the extending primer within the same C flow (e.g., at flow position 3), and signals emitted by the labeled base would have an intensity greater than an intensity level corresponding to a single base incorporation. This is shown in Table 1. The non-binary flowgram also indicates the presence or absence of the base, and can provide additional information including the number of bases likely incorporated into each extending primer at the given flow position. The values do not need to be integers. In some cases, the values can be reflective of uncertainty and/or probabilities of a number of bases being incorporated at a given flow position.

In some instances, the sequencing data set includes flow signals representing a base count indicative of the number of bases in the sequenced nucleic acid molecule that are incorporated at each flow position. For example, as shown in Table 1, the primer extended with a CTG sequence using a T-A-C-G flow cycle order has a value of 1 at position 3, indicating a base count of 1 at that position (the 1 base being C, which is complementary to a G in the sequenced template strand). Also in Table 1, the primer extended with a CCG sequence using the T-A-C-G flow cycle order has a value of 2 at position 3, indicating a base count of 2 at that position for the extending primer during this flow position. Here, the 2 bases refer to the C-C sequence at the start of the CCG sequence in the extending primer sequence, and which is complementary to a G-G sequence in the template strand.

The flow signals in the sequencing data set may include one or more statistical parameters indicative of a likelihood or confidence interval for one or more base counts at each flow position. In some instances, the flow signal is determined from an analog signal that is detected during the sequencing process, such as a fluorescent signal of the one or more bases incorporated into the sequencing primer during sequencing. In some cases, the analog signal can be processed to generate the statistical parameter. For example, a machine learning algorithm can be used to correct for context effects of the analog sequencing signal as described in published International patent application WO 2019084158 A1, which is incorporated by reference herein in its entirety. Although an integer number of zero or more bases are incorporated at any given flow position, a given analog signal many not perfectly match with the analog signal. Therefore, given the detected signal, a statistical parameter indicative of the likelihood of a number of bases incorporated at the flow position can be determined. Solely by way of example, for the CCG sequence in Table 1, the likelihood that the flow signal indicates 2 bases incorporated at flow position 3 may be 0.999, and the likelihood that the flow signal indicates 1 base incorporated at flow position 3 may be 0.001. The sequencing data set may be formatted as a sparse matrix, with a flow signal including a statistical parameter indicative of a likelihood for a plurality of base counts at each flow position. Solely by way of example, a primer extended with a sequence of TATGGTCGTCGA (SEQ ID NO: 1) (that is, the sequencing read reverse complement) using a repeating flow-cycle order of T-A-C-G may result in a sequencing data set shown in FIG. 1A. The statistical parameter or likelihood values may vary, for example, based on the noise or other artifacts present during detection of the analog signal during sequencing. In some instances, if the statistical parameter or likelihood is below a predetermined threshold, the parameter may be set to a predetermined non-zero value that is substantially zero (i.e., some very small value or negligible value) to aid the statistical analysis further discussed herein, wherein a true zero value may give rise to a computational error or insufficiently differentiate between levels of unlikelihood, e.g. very unlikely (0.0001) and inconceivable (0).

A value indicative of the likelihood of the sequencing data set for a given sequence can be determined from the sequencing data set without a sequence alignment. For example the most likely sequence, given the data, can be determined by selecting the base count with the highest likelihood at each flow position, as shown by the stars in FIG. 1B (using the same data shown in FIG. 1A). Thus, the sequence of the primer extension can be determined according to the most likely base count at each flow position: TATGGTCGTCGA (SEQ ID NO: 1). From this, the reverse complement (i.e., the template strand) can be readily determined. Further, the likelihood of this sequencing data set, given the TATGGTCGTCGA (SEQ ID NO: 1) sequence (or the reverse complement), can be determined as the product of the selected likelihood at each flow position.

In some instances, the sequencing data set associated with a nucleic acid molecule is compared to one or more (e.g., 2, 3, 4, 5, 6 or more) possible candidate sequences. A close match (based on match score, as discussed below) between the sequencing data set and a candidate sequence indicates that it is likely the sequencing data set arose from a nucleic acid molecule having the same sequence as the closely matched candidate sequence. In some instances, the sequence of the sequenced nucleic acid molecule may be mapped to a reference sequence (for example using a BWA algorithm or other suitable alignment algorithm) to determine a locus (or one or more loci) for the sequence. The sequencing data set in flowspace can be readily converted to basespace (or vice versa, if the flow order is known), and the mapping may be done in flowspace or basespace. The locus (or loci) corresponding with the mapped sequence can be associated with one or more alternative sequences, which can operate as the candidate sequences (or haplotype sequences) for the analytical methods described herein. One advantage of the methods described herein is that the sequence of the sequenced nucleic acid molecule does not need to be aligned with each candidate sequence using an alignment algorithm in some cases, which is generally computationally expensive. Instead, a match score can be determined for each of the candidate sequences using the sequencing data in flowspace, a more computationally efficient operation.

A match score indicates how well the sequencing data set supports a candidate sequence. For example, a match score indicative of a likelihood that the sequencing data set matches a candidate sequence can be determined by selecting a statistical parameter (e.g., likelihood) at each flow position that corresponds with the base count that flow position, given the expected sequencing data for the candidate sequence. The product of the selected statistical parameter can provide the match score. For example, assume the sequencing data set shown in FIG. 1A for an extended primer, and a candidate primer extension sequence of TATGGTCATCGA (SEQ ID NO: 2). FIG. 1C (showing the same sequencing data set in FIG. 1A) shows a trace for the candidate sequence (solid circles). As a comparison, the trace for the TATGGTCGTCGA (SEQ ID NO: 1) sequence (see FIG. 1B) is shown in FIG. 1C using open circles. The match score indicative of the likelihood that the sequencing data matches a first candidate sequence TATGGTCATCGA (SEQ ID NO: 2) is substantially different from the match score indicative of the likelihood that the sequencing data matches a second candidate sequence TATGGTCGTCGA (SEQ ID NO: 1), even though the sequences vary only by a single base variation. As seen in FIG. 1C, the differences between the traces is observed at flow position 12, and propagates for at least 9 flow positions (and potentially longer if the sequencing data extended across additional flow positions). This continued propagation across one or more flow cycles may be referred to as a “cycle shift,” and is generally a very unlikely event if the sequencing data set matches the candidate sequence.

A small nucleotide variant induces a cycle shift when sequencing data associated with a nucleic acid molecule having the small nucleotide variant shifts relative to reference sequencing data associated with a reference sequence (i.e., a sequence having the same sequence as the nucleic acid molecule except that it does not have the small nucleotide variant) by one or more flow cycles when the nucleic acid sequencing data and the reference sequencing data are sequenced using non-terminating nucleotides provided in separate nucleotide flows according to a flow-cycle order. That is, the sequencing data and the reference sequencing data differ across one or more flow cycles. The reference sequencing data need not be obtained by sequencing a reference nucleic acid molecule, but may be generated in silico based on the reference sequence.

An exemplary cycle shift inducing small nucleotide variant is illustrated by FIG. 1C. Assume the second candidate sequence indicated in FIG. 1C is the sequence read reverse complement TATGGTCGTCGA (SEQ ID NO: 1) associated with the small nucleotide variant-containing nucleic acid molecule (and associated with the sequencing data shown in the flowgram at the top of the figure), and that the first candidate sequence is the sequence read reverse complement TATGGTCATCGA (SEQ ID NO: 2) of the reference sequence. The A to G SNP (at base position 8 of both sequences) induces the cycle shift, which can be observed by the one cycle leftward shift of the sequencing data associated with the small nucleotide variant-containing nucleic acid molecule compared to the reference sequencing data. For example, the T base at base position 9 is sequenced at flow position 13 according to the sequencing data associated with the small nucleotide variant-containing nucleic acid molecule, and at position 17 according to the reference sequencing data. Similarly, the CG bases at base positions 10 and 11 are sequenced at flow positions 15 and 16 according to the sequencing data associated with the small nucleotide variant-containing nucleic acid molecule, and at position 19 and 20 according to the reference sequencing data.

Because a cycle shift event is unlikely in the absence of a true positive event, in some instances, loci from the disease-associated small nucleotide variant panel may be selected only if variants at the loci result in a cycle shift event.

The sensitivity of a short genetic variant to induce a cycle shift can depend on the flow cycle order used to sequence the nucleic acid molecule having the small nucleotide variant. The example illustrated in FIG. 1C included a T-A-C-G flow cycle order, but other flow cycle orders may be used to induce a cycle shift in other variants. The potential of the small nucleotide variant to induce a cycle shift event can be observed using any flow order by the generation of a new zero signal or a new non-zero signal in the sequencing data. Thus, even though the selected flow order did not induce a cycle shift event, the small nucleotide variant can induce a cycle shift event using a different flow order. In some instances, loci from the disease-associated small nucleotide variant panel are selected only if variants at the loci result in the sequencing data and the reference sequencing data differing by the sequencing data having a new zero signal or a new non-zero signal when the nucleic acid sequencing data and the reference sequencing data are sequenced using non-terminating nucleotides provided in separate nucleotide flows according to a flow-cycle order. The signal changes may be consecutive, in some embodiments. In some instances, loci from the disease-associated small nucleotide variant panel are selected only if variants at the loci result in the sequencing data and the reference sequencing data differing at two or more flow positions (which may be consecutive) when the nucleic acid sequencing data and the reference sequencing data are sequenced using non-terminating nucleotides provided in separate nucleotide flows according to the flow-cycle order.

Because the nucleic acid molecule is sequenced using different flow-cycle orders, the sequencing data sets differ. FIG. 1D shows exemplary sequencing data sets for the small nucleotide variant-containing nucleic acid molecule having a reverse complement sequence of TATGGTCGTCGA (SEQ ID NO: 1) determined using a different flow-cycle order (A-G-C-T) than the sequencing data illustrated in FIG. 1C, which was obtained using a T-A-C-G flow cycle). The reference sequencing data is mapped onto the sequencing data for the small nucleotide variant-containing nucleic acid molecule. The small nucleotide variant generates a new zero signal at position 17, and a new non-zero signal at position 18. Thus, even though the T-A-C-G flow cycle induced a cycle shift (see FIG. 1C), the A-G-C-T flow cycle does not, even though the small nucleotide variant is the same. Still, the new zero and new non-zero signals indicate that the small nucleotide variant has the potential to induce a cycle shift using a different cycle order.

### Methods for Detecting Presence, Level, Recurrence, Progression, or Regression of Disease

Determining the fraction of nucleic acid molecules associated with a disease (e.g., a tumor fraction) from a fluidic sample allows for detection of the presence of the disease and/or a determination of the severity of the disease. Diseased tissue, such as tumor, for example, can shed DNA that circulates in the blood of the individual, and the amount of circulating-tumor DNA (ctDNA) within cell-free DNA (cfDNA) is indicative of the presence or severity of the disease. A minimum residual disease level can be indicated when some non-zero (as determined by a applying a selected statistical test) fraction of nucleic acid molecules in a fluidic sample obtained from the individual is detected. This measurement has substantial clinical benefit, for example, after the individual has been treated for the disease and is being monitored for disease recurrence. Disease progression or regression of the disease can also be monitored by observing an increase or decrease (as determined by applying a selected statistical test) of the fraction compared to a prior determined fraction. This can be useful, for example, for evaluating the prognostic benefit of a drug or other therapy administered to the individual.

A unique signature of variants for the diseased tissue (i.e., a personalized disease-associated small nucleotide variant panel) is identified. The small nucleotide variants can be used to discern whether a specific nucleic acid (e.g., cfDNA) molecule originated from the known diseased tissue or not. Sequencing reads that cross an small nucleotide variant locus from the personalized disease-associated small nucleotide variant panel, and optionally pass through one or more filtering steps, can be used to calculate the fraction of nucleic acid molecules. The fraction is related to the number of sequencing reads that support a variant (i.e., associated with the diseased tissue) read or a reference (i.e., associated with non-diseased tissue) according to:

$$F = \frac{{N}^{alt}}{{N}^{alt} + {N}^{ref}} - BG$$

where F is the fraction, N^{alt} is a number of sequencing reads matching the diseased tissue sequence (i.e., alternate read), N^{ref} is a number of sequencing reads matching the normal (non-diseased) sequence (i.e., reference read), and BG is the background false positive error rate.

As further discussed herein, the sequencing read may be a full-length sequencing read or a trimmed sequencing read. The trimmed sequencing read is a fragment of a full-length sequencing read in the sequencing data. A full-length sequencing read, for example, may include more than one (e.g., 2, 3, or more) variant loci from the personalized disease-associated small nucleotide variant panel. Analyzing sequencing reads with a plurality of variant loci can facilitate haplotype mapping, for example determining the likelihood that sequencing read corresponds to a variant sequence haplotype or a reference sequence haplotype. However, this generally generates large data files that are computationally expensive to analyze. Thus, in some implementations and as discussed further herein, a small nucleotide variant-specific likelihood determination may be made using trimmed sequencing reads. The trimmed sequencing read may be trimmed such that it comprises a single variant locus from the disease-associated small nucleotide variant panel (i.e., excludes any other variant locus from the disease-associated small nucleotide variant panel). For example, a variant locus (for example, a locus associated with a single base variant) form the disease-associated small nucleotide variant panel may be identified within the sequencing read, and the sequencing read can be trimmed to comprise the variant locus and exclude any other variant locus from the panel. Thus, a single sequencing read that includes a plurality of variant loci can be trimmed to generate a plurality of trimmed sequencing reads, each having a different variant locus.

As further discussed herein, the background false positive error rate can be minimized by filtering (i.e., excluding) small nucleotide variants that typically have a higher false positive error rate. Sequencing data can also or alternatively be filtered to minimize the false positive error rate, as further described herein. Even with these filtering steps, however, some amount of false positive error will remain, for example, due to a false identification of germline and/or non-disease associated somatic small nucleotide variants as being disease-associated small nucleotide variants, or sequencing errors (e.g., mutations introduced during library preparation prior to sequencing, or other errors introduced during the sequencing and/or calling process).

Sequencing error can be variant-motif specific, particularly when sequencing data is collected using flow-sequencing methods. The methods for determining a level of a disease in an individual can account for variant-motif specific errors using a plurality of variant motif-specific models that each associate sequencing data corresponding to a respective variant motif, a background factor indicative of a false positive error rate for the respective variant motif, and an estimated fraction of the nucleic acid molecules associated with the disease. It has been found that accounting for the false positive error rate using variant motif-specific model, the limit of detection for detecting the fraction of nucleic acid molecules associated with the diseased tissue, with statistical significance, can be substantially reduced.

For example, a method of determining a level of a disease in an individual can include: obtaining sequencing data for nucleic acid molecules obtained from a fluidic sample from the individual, the sequencing data comprising sequencing reads associated with loci selected from a personalized disease-associated small nucleotide variant panel; generating, using the sequencing data, a plurality of variant motif-specific models that each associate sequencing data corresponding to a respective variant motif, a background factor indicative of a false positive error rate for the respective variant motif, and an estimated fraction of the nucleic acid molecules associated with the disease; and determining, from the plurality of variant motif-specific models, a fraction of the nucleic acid molecules associated with the disease for the individual, wherein the fraction indicates the level of the disease in the individual. The sequencing data may be generated, for example, by sequencing the nucleic acid molecules using non-terminating nucleotides provided in separate nucleotide flows according to a flow-cycle order comprising a plurality of flow positions, wherein the flow positions correspond to the nucleotide flows.

Sequencing reads (e.g., full length sequencing reads or trimmed sequencing reads) from the sequencing data obtained for nucleic acid molecules obtained from the fluidic sample (e.g., cfDNA molecules) can be characterized as being an alternate read (i.e. being called as corresponding to a nucleic acid molecule derived from the diseased tissue) or a reference reads (i.e., being called as corresponding to a nucleic acid molecule derived from non-diseased tissue). Optionally, sequencing reads may be characterized as an ambiguous read when not characterized as an alternate read or a reference read. For example, a sequencing read may be characterized as an ambiguous read when the likelihood of the sequencing read being an alternate read or a reference read is below some predetermined likelihood threshold (e.g., a likelihood threshold of about 0.99, about 0.98, about 0.95, about 0.90, about 0.85, or about 0.85). Additionally or alternatively, a sequencing red may be characterized as an ambiguous read if the likelihood of the difference between the likelihood that the respective sequencing read corresponds to an alternative sequence and the likelihood that the respective sequencing read corresponds to a reference sequence is less than a predetermined likelihood difference threshold (e.g., about 3 orders of magnitude or more, about 5 orders of magnitude or more, about 7 orders of magnitude or more, or about 10 orders of magnitude or more). Ambiguous reads may be excluded from the further analysis (e.g., excluded from the fraction determination or the variant-motif specific models).

Some of the sequencing reads characterized as an alternate read may be incorrectly characterized, resulting in a false positive error. A variant motif-specific model can be generated for each of a plurality of variant motifs, which can be used to correct for the false positive error rate for the respective variant motif when determining the fraction of nucleic acid molecules associated with the disease. Sequencing data for nucleic acid molecules obtained from a plurality of control individuals may be used as a basis for the background factor. The control individuals may be healthy individuals or individuals with a disease (e.g., a tumor), which may be the same type of disease (or same type of tumor) as the tested individual. The small nucleotide variant signature of the disease is personalized; therefore, small nucleotide variants for the disease of the test individual will be different (and will rarely overlap, if at all) with the small nucleotide variants for the disease of the control individual. Further, the false positive error rate for the same variant motif can be assumed to be the same in the control individuals and the test individual. This is especially true when the sequencing data for the nucleic acid molecules of the test individual and the sequencing data for the nucleic acid molecules for the plurality of control individuals are simultaneously obtained in a pooled sample. Thus, the sequencing data for nucleic acid molecules obtained from a plurality of control individuals used for the motif-specific variant model can include sequencing reads associated with loci selected from the personalized disease-associated small nucleotide variant panel.

Variant motifs provide a context for any given variant in the personalized disease-associated small nucleotide variant panel, and includes the variant basis along with one or more bases flanking the 5′ end of the variant and one or more bases flanking the 3′ end of the variant. Both the reference sequence and the alternative sequence are collectively considered the variant motif. For example, a SNP includes a single nucleotide variant, and the variant motif can include the single nucleotide position itself and one or more bases flanking the 5′ end of the SNP and one or more bases flanking the 3′ end of the SNP. The variant motif can be, for example, a trinucleotide SNP variant motif. 192 different trinucleotide SNP variant motifs are possible, and the variant-specific models can include, for example, one model for each of the different trinucleotide SNP variant motifs. Variant motifs may be longer than 3 bases in length, for example about 4, 5, 6, 7, 8, 9, 10, 11 or more bases in length. The same variant motif may occur at multiple different loci within the personalized disease-associated small nucleotide variant panel, and the plurality of variant motif-specific models allow for a background false positive error rate analysis for variant motifs across loci associated with a common variant motif.

The variant motif-specific model can relate the sequencing data corresponding the respective motif, m, to the background factor indicative of a false positive error rate for the respective variant motif, BGₘ, for the motif and the fraction, F, of nucleic acid molecules associated with the disease according to:

Nₘ^{alt}=(F+BGₘ)Nₘ^{total},

wherein Nₘ^{alt} is a number of alternative sequencing reads comprising a locus corresponding to variant motif m and Nₘ^{total} is a total number of sequencing reads comprising a locus corresponding to variant motif m. While the background factor is variant-motif specific, the fraction F is constant across all variant motif-specific models.

For each variant motif-specific model, a statistical value indicative of a likelihood of the sequencing data fitting the model can be determined for each of a plurality of different fractions. A most likely fraction, given the statistical values for each variant motif, can then be determined to establish the fraction for the individual. The statistical value may be, for example, the likelihood value itself, a log-likelihood value, or any other similar parameter that indicates the likelihood.

The variant motif-specific model can be, for example, a binomial distribution of the sequencing reads comprising a locus corresponding to the respective variant motif. The probability pₘ, of observing an alternative sequencing read comprising a locus corresponding to variant motif m can be defined by:

$${p}_{m} = \frac{{N}_{m}^{alt}}{{N}_{m}^{total}} = F + {BG}_{m}$$

That is, the probability of the distribution is a motif-specific fraction estimate. The true mean of the binomial distribution can be defined by:

Nₘ^{alt}=(F+BGₘ)Nₘ^{total}

The plurality of variant motif-specific models can then be fit to determine the most likely fraction across all models. For example, for a given estimated fraction, likelihood of the sequencing reads comprising a locus corresponding to the respective variant motif-specific model can be determined. Log-likelihoods across the plurality of variant motif-specific models can be summed, and a maximum likelihood estimate for the fraction can be determined. The fraction that yields the maximum likelihood estimate can then be deemed the fraction of nucleic acid molecules associated with the disease for the individual.

In another example, the fraction for the individual can be determined by determining, for each variant motif-specific model, a statistical value indicative of a likelihood of each of a plurality of estimated fractions, given the sequencing data corresponding to the variant motif and control sequencing data for nucleic acid molecules obtained from one or more control fluidic samples. The control fluidic samples can be obtained from healthy individuals or individuals with a similar disease but with a different small nucleotide variant signature for the disease. An alternate read that supports the presence of an small nucleotide variant from the personalized disease-associated small nucleotide variant panel that is included in the control sequencing data is attributed to background noise, and is indicative of the false positive error rate. That is, the distribution of reads in control sequencing data indicates a background distribution that arises a fraction of zero, whereas the distribution of reads in the sequencing data of the individual arises from the background distribution plus an unknown fraction. The likelihood that the distribution of sequencing reads in the sequencing data (corresponding to the variant motif) for the individual is the same as (or differs from) the distribution of sequencing reads in the control sequencing data (corresponding to the variant motif) can be determined using a selected statistical test. The statistical test may be, for example, an exact test (e.g., a Fisher exact test) or a non-exact test (e.g., a Chi-squared test).

The likelihood that the distribution of sequencing reads in the sequencing data (corresponding to the variant motif) for the individual is the same as (or differs from) the distribution of sequencing reads in the control sequencing data (corresponding to the variant motif) can be determined for a plurality of different estimated fractions. The initial control sequencing data assumes a fraction of zero, but the control sequencing data can be adjusted for one or more non-zero estimated fractions. A random binomial sample from the distribution of sequencing in the control sample can be generated for a non-zero tumor fraction using a random realization method (e.g., a Monte Carlo method) with a distribution probability equal to the respective estimated non-zero fraction. The likelihood that the distribution of sequencing reads in the sequencing data (corresponding to the variant motif) for the individual is the same as (or differs from) the distribution of sequencing reads in the adjusted control sequencing data (corresponding to the variant motif) for the non-zero estimated tumor fraction can be determined using the same selected statistical test.

By way of example, the variant motif-specific model can include a 2×2 contingency table with a number of alternate reads or reference reads (columns) in the sequencing data for the individual or in the control sequencing data (rows). The initial distribution of sequencing reads control sequencing data assumes a fraction of zero, and the distribution is due to background noise alone. A new control sequencing data distribution for a non-zero estimated fraction can be generated by randomly moving sequencing read counts from the reference column to the alternate read column according to a probability equal to the respective estimated non-zero fraction. The likelihood that the distribution of sequencing reads in the sequencing data (corresponding to the variant motif) for the individual is the same as (or differs from) the distribution of sequencing reads in the adjusted control sequencing data (corresponding to the variant motif) for the non-zero estimated tumor fraction can be determined using the same selected statistical test. Because the distribution is random, there is a chance that the resulting distribution could be biased. To correct for this, a plurality of likelihood valued may be obtained using a plurality of random realizations (e.g., 2, 3, 4, 5, 6, 7, 8, 9, 10 or more) for a given tumor fraction (each with a distribution probability equal to the respective non-zero estimated fraction). The average likelihood for the plurality of random realizations can be taken as the statistical value.

The statistical value indicative of the likelihood of a given estimated fraction, given the sequencing data for the individual and the control sequencing data for each variant motif-specific model can be merged to determine a statistical value indicative of the likelihood of a given estimated fraction for the individual and the control sequencing data across the plurality of variant motif-specific models. For example, the log-likelihoods of each variant motif-specific model, for a given estimated fraction, can be summed to determine a log-likelihood of the estimated fraction being the true faction for the individual. A maximum likelihood estimate can then be made to determine, from the plurality of estimated fractions, the fraction of the nucleic acid molecules associated with the disease.

The determined fraction may be reported on its own, for example as a level of disease in the individual, or may be reported as a statistically significant different from a background level. For example, a statistical test may be used to determine if the determined fraction for the individual is greater than a background level with statistical significance. The specific test and the significance threshold can be determined by one skilled in the art, for example based on the desired confidence in the determination. By way of example, a Z-score can be determined to discern whether the determined tumor fraction is greater than a fraction of zero. The log-likelihood of the sequencing data for the individual given a fraction of zero can be subtracted from the log-likelihood of the sequencing data for the individual given the determined fraction, and the resulting value divided by the standard deviation of the log-likelihood of the sequencing data for the individual given the determined fraction. The standard deviation of the log-likelihood of the sequencing data for the individual given the determined fraction is determined, for example, using the plurality of likelihood values obtained using a plurality of randomization where the distribution probability is equal to the determined fraction. The significance threshold can be set as desired, for example a Z-score of about 4 or higher, about 5 or higher, about 6 or higher, about 7 or higher, about 8 or higher, about 9 or higher, or about 10 or higher.

Small nucleotide variant-specific determination. In some instances, instead of evaluating sample information based on haplotype likelihoods, samples may be evaluated based on sets of individual small nucleotide variants. That is, in some implementations, the method may exclude haplotypes that cover multiple small nucleotide variants and/or include complex mutations (e.g., indels, inversions, translocations, etc.). By considering only small nucleotide variants, it is easier to determine an overall background level because each locus in the genome may be evaluated as a separate small nucleotide variant. The small nucleotide variant-specific analysis is modular (e.g., the somatic variant calling is performed separately from determining the cfDNA feature-map (e.g., disease-specific features)).

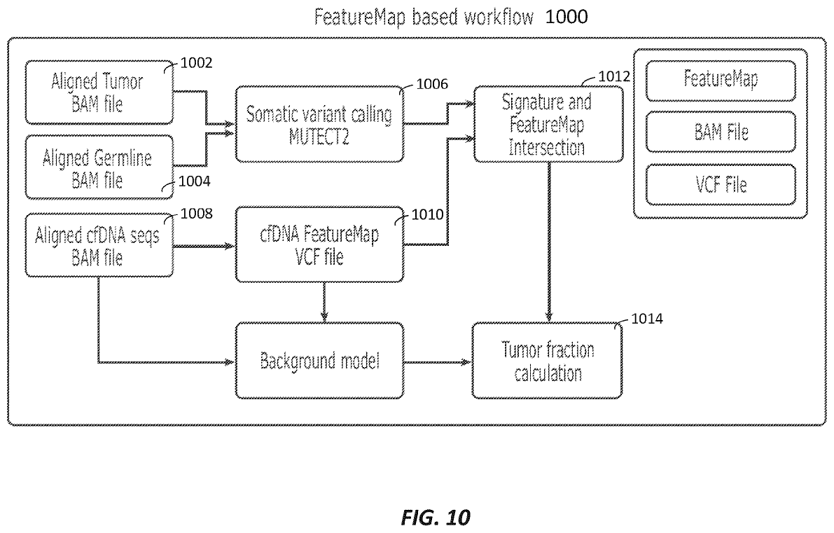

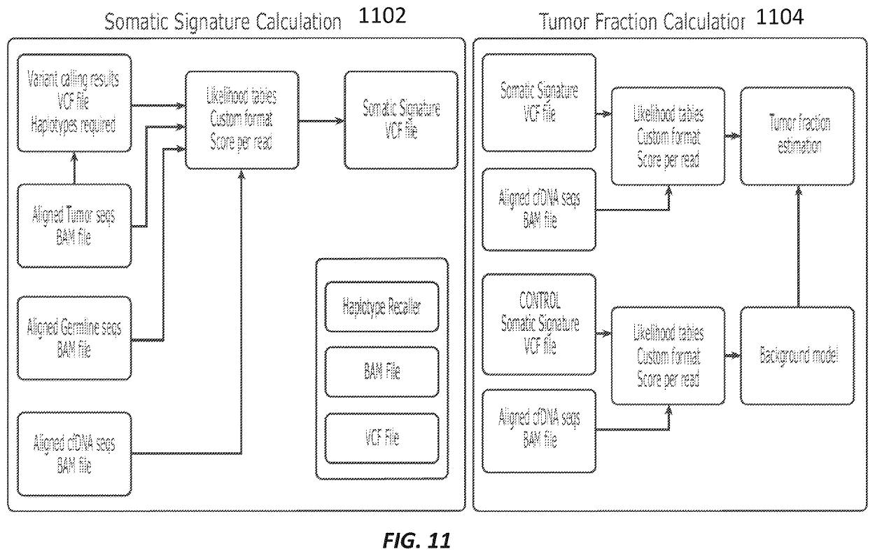

The small nucleotide variant-specific method is outlined in FIG. 10 and includes the following steps. Matched tumor (e.g., aligned tumor 1002) and normal (e.g., aligned germline 1004) samples are compared to generate a somatic variant calls dataset 1006. In some instances, using matched samples can decrease the likelihood of incorporating irrelevant artefacts in the analysis. A feature map of small nucleotide variants 1010 can further be extracted directly from cfDNA sequencing data 1008. The separation of the cfDNA 1008 from the determination of somatic variant calls 1006 for an individual makes it easier to analyze additional samples for the same individual (e.g., over multiple timepoints). In addition, the feature map is, in some implementations, more efficiently stores subject-specific small nucleotide variant information (e.g., as compared with the method outlined in FIG. 11). This is due to the fact that each sequencing read analyzed for each small nucleotide variant is trimmed substantially (e.g., to a predetermined number of bases upstream and downstream of the small nucleotide variant). The intersection of the somatic variant calls 1006 and the cfDNA feature map 1012 can be used to determine an estimated tumor fraction for a subject 1014.

In some instances, external somatic variant calling results can be used instead of aligned tumor-normal variant calls 1006. For example, the external somatic variant calls may comprise a targeted set of small nucleotide variants. In another example, the tumor and normal data may be obtained from a different subject from the cfDNA or may be obtained from the same subject but at a different time than when the cfDNA was obtained.

As discussed elsewhere herein, a plurality of quality metrics can be used to filter the sequence reads used for analysis (e.g., both for the somatic variant calling and the cfDNA feature map). A set of example filters that can be used to exclude reads from analysis based on different quality metrics are described below.

In some instances, one filter may be used may be used to exclude sequence reads from downstream analysis. In some instances, more than one filter may be used to exclude sequence reads from analysis (e.g., a combination of any two or more filters described herein). In some instances, a plurality of filters described herein may be used to filter reads. In some instances, there is a predetermined order of applying the one or more filters. In some instances, each filter in the one or more filters is independent, and the one or more filters can be applied in any desired order.

X-SCORE refers to a score for the small nucleotide variant sequencing accuracy. This is effectively the base quality, defined as (−log₁₀*pₑᵣᵣₒᵣ). In some instances, a sequencing accuracy threshold is set at a log-likelihood of greater than 5. In some instances, a sequencing accuracy threshold is set at a log-likelihood of greater than 10. The X-SCORE is, in some instances, the most important output used to filter reads (e.g., filters out the most sequencing reads). In some instances, the minimum value is 3 (lesser small nucleotide variants are not reported) and the maximum is 10 (only cycle skip small nucleotide variants can reach those values).

X-MAPQ refers to read mapping quality. In typical analyses, the only allowed value is MAPQ=60. In some instances, another threshold can be set, or a range can be used to filter reads by MAPQ values.

X-EDIST refers to the edit distance (e.g., Levenshtein distance) of the read from the reference. The edit distance may be calculated using a variety of approaches. In some instances, the edit distance can be calculated by counting (e.g., using the at least one processor), a number of different elements between the read and the reference. In some instances, the edit distance may be calculated in basespace. In some instances, the edit distance may be calculated in flowspace (e.g., using a flowspace rendition of the reference sequence). In some instances, the edit distance may be any useful edit distance, e.g., a Levenshtein distance, a longest common subsequence distance, a Hamming distance, a Jardo distance, a Damerau-Levenshtein distance, or analogs or derivatives thereof. In an example, a Hamming distance may be calculated between the read and the reference. In such an example, each position (e.g., element, which may comprise a base call or a flow cycle value (e.g., H-mer)) of the reference is compared to the corresponding position in the read. If the values differ for a given position, a value of 1 distance unit is added (e.g., every position that differs increases the value of the edit distance by 1). Each position between the read and the reference that do not differ in value does not increase the edit distance.

An edit distance threshold (e.g., to determine that a read comprises a variant as compared with the reference) may be set at any useful value. In some instances, the edit distance threshold may be at least 1, at least 2, at least 3, at least 4, at least 5, at least 6, at least 7, at least 8, at least 9, at least 10, or more distance units between a read and a reference sequence. In other instances, a maximum edit distance threshold may be set, e.g., at most 10, at most 9, at most 8, at most 7, at most 6, at most 5, at most 4, at most 3, at most 2, or at most 1 distance units between a read and a reference sequence.

X-FC1 refers to a number of features (small nucleotide variants) present on the same read. A single read may cover multiple small nucleotide variants at different locations. Thus, a single read can be reported multiple times for multiple small nucleotide variants. However, each small nucleotide variant is analyzed independently. X-FC2 refers to a number of features (small nucleotide variants) on the same read that passed any of the filters used (e.g., matching the reference for +/−5 bases).

X-READ-COUNT refers to the coverage at the position. X-FILTERED-COUNT refers to the coverage in the position only for reads that passed any filters used filter (e.g., matching the reference for −/+5 bases). The ratio of X-FILTERED-COUNT/X-READ-COUNT is the ratio of filtration use in the small nucleotide variant-specific method; this depends on sample and on input parameters and should be accounted for when calculating the effective coverage for any one small nucleotide variant.

X-FLAGS is a value propagated from the BAM file flag. Since there is stringent filtration in the FeatureMaps tool the only flag options are 0/16 for forward/reverse orientation.

X-LENGTH refers to the read length after adapter trimming. In some instances, X-LENGTH depends on a cohort of subjects analyzed, a protocol used to extract samples from a subject, a sequencing protocol, a cancer type, etc.

X-CIGAR is a value propagated from the BAM file. In some instance, the X-CIGAR value can be used to remove reads with too many clipped bases.

RQ refers to a sequencing quality metric for the read. Lower values indicate higher quality reads. Generally, an RQ value is used for filtration during base calling.

In some instances, the small nucleotide variant-specific method retains or provides additional information for each small nucleotide variant.

The methods described herein may be useful for detecting the presence (such as recurrence) of a disease, measuring a level of the disease, or measuring or detecting a progression or regression of the disease. In some instances of the methods described herein, the individual has been previously treated for the disease. In some instances, the disease is suspected to be in remission, such as complete remission or partial remission. After treatment of the disease, for example by chemotherapy or excision of a cancer, the disease may recur, for example due to incomplete removal or killing of all diseased tissue. A cancer, for example, may metastasize and relocate at a different position in the individual, or may be too small to be detected by known imaging modalities (e.g., MRI, PET scan, etc.). Monitoring the individual for recurrence, regression, or progression of the disease might be done periodically so that the individual can be retreated if the disease recurs or progresses.

The presence or residual level of the disease (e.g., cancer) can be detected, for example, by comparing, using nucleic acid sequencing data associated with the individual, a signal indicative of a rate at which sequenced loci selected from a personalized disease-associated small nucleotide variant panel are derived from a diseased tissue to a noise factor indicative of a sampling variance across the selected loci; and determining whether the individual has the disease based on the comparison of the signal to the background factor. In some instances, the signal-to-noise ratio is determined, for example as described herein.

FIG. 2 shows an exemplary method of measuring a level of a disease (such as a tumor) in an individual. Sequencing data for nucleic acid molecules (e.g., cfDNA molecules) obtained from a fluidic sample (e.g., a blood sample, a plasma sample, a saliva sample, a urine sample, or a fecal sample) from the individual is obtained at step 205. The sequencing data may be generated, for example, by sequencing the nucleic acid molecules using non-terminating nucleotides provided in separate nucleotide flows according to a flow-cycle order comprising a plurality of flow positions, wherein the flow positions correspond to the nucleotide flows. Optionally, the nucleic acid sequencing data is untargeted and/or unenriched nucleic acid sequencing data (such as whole-genome sequencing data). The sequencing data is obtained without the use of unique molecular identifiers (UMIs). In some instances, the sequencing depth of the sequencing data is less than about 100, less than about 10, or less than about 1. In some instances, the sequencing depth of the sequencing data is at least 0.01.

The sequencing data includes sequencing reads associated with loci selected from a personalized disease-associated small nucleotide variant panel. The personalized disease-associated small nucleotide variant panel may be determined apriori, or may be selected from an initial small nucleotide variant panel using the sequencing data. For example, one or more small nucleotide variants in the small nucleotide variant panel may be excluded from analysis. In FIG. 2, generation of the personalized disease-associated small nucleotide variant panel is shown at 210 after the sequencing data is obtained, although in some instances the small nucleotide variant panel is generated prior to obtaining the sequencing data. The personalized disease-associated small nucleotide variant panel includes small nucleotide variants that indicate the variant signature of the disease. For example, nucleic acid molecules derived from a diseased tissue sample (e.g., a tumor biopsy) can be sequenced, and variant calls can be made. Optionally, nucleic acid molecules from a non-diseased tissue (e.g., a buffy coat, white blood cells, peripheral blood mononuclear cells) can be sequenced, and germline variant or non-disease associated somatic variant calls can be made. The small nucleotide variants from the diseased tissue can be filtered to exclude germline variants and/or non-disease associated somatic variants. Other filtering methods, such as those discussed herein, may be employed to select small nucleotide variants with a low false positive error rate. For example, the small nucleotide variant panel may be filtered such that at least 90% of small nucleotide variants in the personalized disease-associated small nucleotide variant panel are associated with small nucleotide variant sequencing data that differs from reference sequencing data associated with a reference sequence across one or more flow cycles when the sequencing data and the reference sequencing data are sequenced using non-terminating nucleotides provided in separate nucleotide flows according to the flow-cycle order.

The small nucleotide variants in the personalized disease-associated small nucleotide variant panel are characterized by a specific variant motif. In some instances, a plurality of variant motif-specific models are generated at step 215 using the sequencing data for the individual. Each variant motif-specific model associated sequencing data corresponding to a respective variant motif, a background factor indicative of a false positive error rate for the respective variant motif, and an estimated fraction (e.g., a tumor fraction) of the nucleic acid molecules associated with the disease. In some instances, at step 220, a fraction of the nucleic acid molecules associated with the disease for the individual is determined from the plurality of variant motif-specific models. The fraction indicates the level of the disease in the individual.

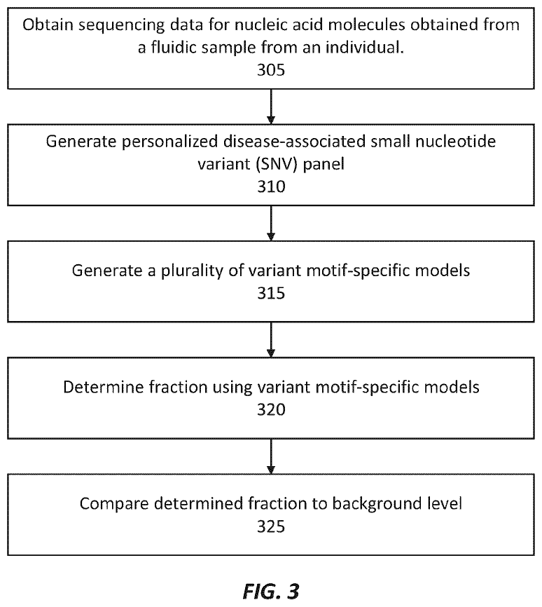

FIG. 3 shows an exemplary method of determining a presence or absence of a disease (such as a tumor) in an individual. Sequencing data for nucleic acid molecules (e.g., cfDNA molecules) obtained from a fluidic sample (e.g., a blood sample, a plasma sample, a saliva sample, a urine sample, or a fecal sample) from the individual is obtained at 305. The sequencing data may be generated, for example, by sequencing the nucleic acid molecules using non-terminating nucleotides provided in separate nucleotide flows according to a flow-cycle order comprising a plurality of flow positions, wherein the flow positions correspond to the nucleotide flows. Optionally, the nucleic acid sequencing data is untargeted and/or unenriched nucleic acid sequencing data (such as whole-genome sequencing data). The sequencing data is obtained without the use of unique molecular identifiers (UMIs). In some instances, the sequencing depth of the sequencing data is less than about 100, less than about 10, or less than about 1. In some instances, the sequencing depth of the sequencing data is at least 0.01.

The sequencing data includes sequencing reads associated with loci selected from a personalized disease-associated small nucleotide variant panel. The personalized disease-associated small nucleotide variant panel may be determined apriori, or may be selected from an initial small nucleotide variant panel using the sequencing data. For example, one or more small nucleotide variants in the small nucleotide variant panel may be excluded from analysis. In FIG. 3, generation of the personalized disease-associated small nucleotide variant panel is shown at 310 after the sequencing data is obtained, although in some instances the small nucleotide variant panel is generated prior to obtaining the sequencing data. The personalized disease-associated small nucleotide variant panel includes small nucleotide variants that indicate the variant signature of the disease. For example, nucleic acid molecules derived from a diseased tissue sample (e.g., a tumor biopsy) can be sequenced, and variant calls can be made. Optionally, nucleic acid molecules from a non-diseased tissue (e.g., a buffy coat, white blood cells, peripheral blood mononuclear cells) can be sequenced, and germline variant or non-disease associated somatic variant calls can be made. The small nucleotide variants from the diseased tissue can be filtered to exclude germline variants and/or non-disease associated somatic variants. Other filtering methods, such as those discussed herein, may be employed to select small nucleotide variants with a low false positive error rate. For example, the small nucleotide variant panel may be filtered such that at least 90% of small nucleotide variants in the personalized disease-associated small nucleotide variant panel are associated with small nucleotide variant sequencing data that differs from reference sequencing data associated with a reference sequence across one or more flow cycles when the sequencing data and the reference sequencing data are sequenced using non-terminating nucleotides provided in separate nucleotide flows according to the flow-cycle order.

The small nucleotide variants in the personalized disease-associated small nucleotide variant panel are characterized by a specific variant motif. At 315 a plurality of variant motif-specific models are generated using the sequencing data for the individual. Each variant motif-specific model associated sequencing data corresponding to a respective variant motif, a background factor indicative of a false positive error rate for the respective variant motif, and an estimated fraction (e.g., a tumor fraction) of the nucleic acid molecules associated with the disease. At step 320 a fraction of the nucleic acid molecules associated with the disease for the individual is determined from the plurality of variant motif-specific models. At step 325, the fraction is compared to a background level. The presence of the disease in the individual is detected if the fraction is above the background level (e.g., with statistical significance).

Optionally, the measured fraction, measured level, progression, regression, and/or recurrence of the disease is recorded in a record, such as an electronic medical record (EMR) or patient file. In some instances of any of the methods described herein, the individual is informed of the measured fraction, measured level, progression, regression, and/or recurrence of the disease. In some instances of any of the methods described herein, the individual is diagnosed with the disease, a recurrence of the disease, or a progression of the disease. In some instances of any of the methods described herein, the individual is treated for the disease based at least in part on the measured fraction, measured level, progression, regression, and/or recurrence of the disease.

### Systems and Devices

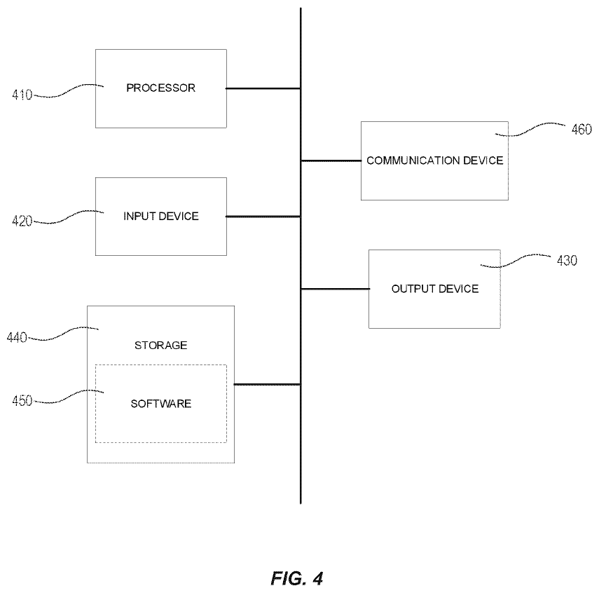

The operations described above, including those described with reference to FIGS. 1-2, are optionally implemented by components depicted in FIG. 4. It would be clear to a person of ordinary skill in the art how other processes, for example, combinations or sub-combinations of all or part of the operations described above, may be implemented based on the components depicted in FIG. 4. It would also be clear to a person having ordinary skill in the art how the methods, techniques, systems, and devices described herein may be combined with one another, in whole or in part, whether or not those methods, techniques, systems, and/or devices are implemented by and/or provided by the components depicted in FIG. 4.

FIG. 4 illustrates an example of a computing device in accordance with one embodiment. Device or system 400 can be a host computer connected to a network. Device 400 can be a client computer or a server. As shown in FIG. 4, device 400 can be any suitable type of microprocessor-based device, such as a personal computer, workstation, server, or handheld computing device (portable electronic device) such as a phone or tablet. The device can include, for example, one or more of the devices: processor(s) 410, input device 420, output device 430, storage 440 (e.g., persistent and/or non-persistent memory), and communication 460 (e.g., one or more network interfaces). Input device 420 and output device 430 can generally correspond to those described above, and can either be connectable or integrated with the computer.

Input device 420 can be any suitable device that provides input, such as a touch screen, keyboard or keypad, mouse, or voice-recognition device. Output device 430 can be any suitable device that provides output, such as a touch screen, haptics device, or speaker.

Storage 440 can be any suitable device that provides storage, such as an electrical, magnetic or optical memory including a RAM, cache, hard drive, or removable storage disk. Communication device 460 can include any suitable device capable of transmitting and receiving signals over a network, such as a network interface chip or device. The components of the computer can be connected in any suitable manner, such as via a physical bus or wirelessly.

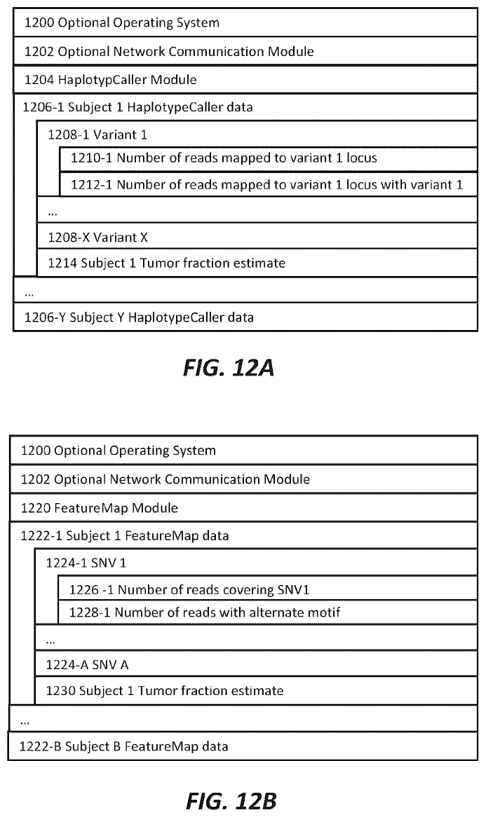

Software 450, which can be stored in storage 440 and executed by processor 410, can include, for example, the programming that embodies the functionality of the present disclosure (e.g., as embodied in the devices as described above). In some implementations, storage 440 comprises non-transitory computer readable storage medium. In some implementations, as illustrated in FIG. 12A, storage 440 stores the following programs, modules, and data structures (e.g., software 450), or a subset thereof:

Optional operating system 1200 which includes procedures for handling various basic system services and for performing hardware-dependent tasks;

Optional network communication module (or instructions) 1202 for connecting system 400 with other devices or with a communication network;

HaplotypeCaller module 1204 for providing a tumor fraction estimation for a subject; and

Information for a subject 1206 in a plurality of subjects including, i) for each variant 308, a respective number of reads 1210 mapped to the corresponding variant locus in a reference sequence and a respective number of mapped reads with the variant 1212, and ii) a subject-specific tumor fraction estimation based at least in part on the respective percentage of variant reads 1212 in the total number of mapped reads 1210 for each variant motif 1208.

In some implementations, as illustrated in FIG. 12B, storage 440 stores the following programs, modules, and data structures (e.g., software 450), or a subset thereof:

Optional operating system 1200 which includes procedures for handling various basic system services and for performing hardware-dependent tasks;

Optional network communication module (or instructions) 1202 for connecting system 400 with other devices or with a communication network;

FeatureMap module 1220 for providing a tumor fraction estimation for a subject; and

Information for a subject 1222 in a plurality of subjects including i) for each small nucleotide variant 1224, a respective number of reads mapped to the corresponding small nucleotide variant in a reference sequence 1226 and a respective number of mapped reads with the alternative motif for the corresponding small nucleotide variant 1228, and ii) a subject-specific tumor fraction estimation based at least in part on the respective percentage of variant reads 1228 in the total number of mapped reads 1226 for each small nucleotide variant 1224.

In various implementations, one or more of the above identified elements are stored in one or more of the previously mentioned memory devices, and correspond to a set of instructions for performing a function described above. The above identified modules, data, or programs (e.g., sets of instructions) need not be implemented as separate software programs, procedures, datasets, or modules, and thus various subsets of these modules and data may be combined or otherwise re-arranged in various implementations (e.g., one or more modules or data sets outlined in FIG. 12B may be stored with one or more of the modules or data sets outlined in FIG. 12A). In some implementations, for example, a tumor fraction estimation may be calculated for a same subject using the modules, data, or programs in FIG. 12A and using the modules, data, or programs in FIG. 12B. In some implementations, non-persistent memory optionally stores a subset of the modules and data structures identified above. Furthermore, in some implementations, the memory stores additional modules and data structures not described above. In some implementations, one or more of the above identified elements is stored in another computer system.

Software 450 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 440, that can contain or store programming for use by or in connection with an instruction execution system, apparatus, or device.

Software 450 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

Device 400 may be connected to a network (e.g., via communication device 460), which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 400 can implement any operating system suitable for operating on the network. Software 450 can be written in any suitable programming language, such as C, C++, Java or Python. In various instances, application software embodying the functionality of the present disclosure can be deployed in different configurations, such as in a client/server arrangement or through a Web browser as a Web-based application or Web service, for example.

The methods described herein optionally further include reporting information determined using the analytical methods and/or generating a report containing the information determined suing the analytical methods. For example, in some instances, the method further includes reporting or generating a report containing related to the level of disease in the individual. Reported information or information within the report may be associated with, for example, a fraction of cfDNA in a sample obtained from the individual that is attributable to a disease (such as a cancer), or the presence or absence of a detectable amount of disease (such as cancer). The report may be distributed to or the information may be reported to a recipient, for example a clinician, the subject, or a researcher.

### EXEMPLARY EMBODIMENTS

The following embodiments are exemplary and are not intended to limit the scope of the claimed invention.

1. A method of determining a level of a disease in an individual, comprising:
obtaining sequencing data for nucleic acid molecules obtained from a fluidic sample from the individual, the sequencing data comprising sequencing reads associated with loci selected from a personalized disease-associated small nucleotide variant (SNV) panel;
generating, using the sequencing data, a plurality of variant motif-specific models that each associate sequencing data corresponding to a respective variant motif, a background factor indicative of a false positive error rate for the respective variant motif, and an estimated fraction of the nucleic acid molecules associated with the disease; and
determining, from the plurality of variant motif-specific models, a fraction of the nucleic acid molecules associated with the disease for the individual, wherein the fraction indicates the level of the disease in the individual.

2. The method of embodiment 1, wherein the level of disease in the individual is a presence or absence of the disease.

3. The method of embodiment 1, wherein the level of disease in the individual is a quantitative value indicating the severity of the disease.

4. A method of determining a presence or absence of a disease in an individual, comprising:
obtaining sequencing data for nucleic acid molecules obtained from a fluidic sample from the individual, the sequencing data comprising sequencing reads associated with loci selected from a personalized disease-associated small nucleotide variant (SNV) panel;
generating, using the sequencing data, a plurality of variant motif-specific models that each associate sequencing data corresponding to a respective variant motif, a background factor indicative of a false positive error rate for the respective variant motif, and an estimated fraction of the nucleic acid molecules associated with the disease; and
determining, from the plurality of variant motif-specific models, a fraction of the nucleic acid molecules associated with the disease for the individual; and
comparing the fraction to a background level, wherein the fraction being above the background level indicates the presence of the disease in the individual.

5. The method of any one of embodiments 1-4, wherein the sequencing data is generated by sequencing the nucleic acid molecules using non-terminating nucleotides provided in separate nucleotide flows according to a flow-cycle order comprising a plurality of flow positions, wherein the flow positions correspond to the nucleotide flows.

6. The method of any one of embodiments 1-4, wherein obtaining the sequencing data comprises sequencing the nucleic acid molecules using non-terminating nucleotides provided in separate nucleotide flows according to a flow-cycle order comprising a plurality of flow positions, wherein the flow positions correspond to the nucleotide flows.

7. The method of any one of embodiments 1-6, further comprising:
for each of the sequencing reads, determining a likelihood that the sequencing read corresponds to a variant sequence and a likelihood that the sequencing read corresponds to a reference sequence, and
for a respective sequence read, if the difference between the likelihood that the respective sequencing read corresponds to the variant sequence and the likelihood that the respective sequencing read corresponds to a reference sequence is less than a predetermined likelihood difference threshold, then excluding sequencing data corresponding to the respective sequencing read from the plurality of variant motif-specific models.

8. The method of embodiment 7, wherein the variant sequence and the reference sequence are corresponding haplotype sequences.

9. The method of embodiment 7 or 8, wherein the variant sequence and the reference sequence differ by at least two bases.

10. The method of embodiment 7 or 8, wherein the variant sequence and the reference sequence comprise at least two loci from the personalized disease-associated SNV panel.

11. The method of any one of embodiments 1-6, further comprising, for each of the sequencing reads:
identifying a variant locus from the personalized disease-associated SNV panel within the sequencing read, wherein the variant locus is associated with a single base variant;
trimming the sequencing read to generate a trimmed sequencing read comprising the variant locus and excluding any other variant locus from the personalized disease-associated SNV panel;
determining a likelihood that the trimmed sequencing read corresponds to a variant sequence comprising and a likelihood that the sequencing read corresponds to a reference sequence, wherein the variant sequence and the reference sequence each comprises the variant locus and excludes any other variant locus from the personalized disease-associated SNV panel; and
for a respective trimmed sequencing read, if the difference between the likelihood that the respective trimmed sequencing read corresponds to the variant sequence and the likelihood that the respective trimmed sequencing read corresponds to a reference sequence is less than a predetermined likelihood difference threshold, then excluding sequencing data corresponding to the respective trimmed sequencing read from the plurality of variant motif-specific models.

12. The method of any one of embodiments 7-11, wherein the predetermined likelihood difference threshold is set at a value of 5 orders of magnitude or higher.

13. The method of any one of embodiments 1-12, wherein at least 90% of SNVs in the personalized disease-associated SNV panel are associated with SNV sequencing data that differs from reference sequencing data associated with a reference sequence at two or more flow positions, wherein the SNV sequencing data and the reference sequencing data are sequenced using non-terminating nucleotides provided in separate nucleotide flows according to a flow-cycle order comprising a plurality of flow positions, wherein the flow positions correspond to nucleotide flows.

14. The method of any one of embodiments 1-13, wherein at least 90% of SNVs in the personalized disease-associated SNV panel are associated with SNV sequencing data that differs from reference sequencing data associated with a reference sequence across one or more flow cycles in a flow-cycle order, wherein the sequencing data and the reference sequencing data are sequenced using non-terminating nucleotides provided in separate nucleotide flows according to the flow-cycle order.

15. The method of any one of embodiments 1-14, further comprising characterizing the sequencing reads as an alternate read, a reference read, or an ambiguous read, wherein a sequencing read characterized as an ambiguous read is excluded from the plurality of variant motif-specific models.

16. The method of any one of embodiments 1-15, wherein the plurality of variant motif-specific models comprises a respective variant motif-specific model for each of a plurality of trinucleotide SNP motifs.

17. The method of embodiment 16, wherein the plurality of variant motif-specific models comprises 192 trinucleotide SNP variant motif-specific models.

18. The method of any one of embodiments 1-17, wherein each variant motif-specific model associates the sequencing data corresponding to variant motif, m, to the background factor, BGₘ, and the estimated faction, F, according to:

Nₘ^{alt}=(F+BGₘ)Nₘ^{total},

wherein Nₘ^{alt} is a number of alternative sequencing reads comprising a locus corresponding to variant motif m and Nₘ^{total} is a total number of sequencing reads comprising a locus corresponding to variant motif m.

19. The method of any one of embodiments 1-18, wherein each variant motif-specific model is a binomial distribution of the sequencing reads comprising a locus corresponding to variant motif m, with a probability, pₘ, of observing an alternative sequencing read comprising a locus corresponding to variant motif m based on pₘ=F+BGₘ, wherein F is the estimated fraction, and BGₘ is the background factor.

20. The method of any one of embodiments 1-19, wherein determining the fraction for the individual comprises determining a maximum likelihood estimate for the fraction given the plurality of variant motif-specific models.

21. The method of any one of embodiments 1-20, wherein determining the fraction for the individual comprises:
determining, for each variant motif-specific model, a statistical value indicative of a likelihood of each of a plurality of estimated fractions, given the sequencing data for the nucleic acid molecules obtained from the fluidic sample from the individual corresponding to the respective variant motif, and
determining a most likely fraction given the statistical values for each variant motif.

22. The method of embodiment 21, wherein each variant motif-specific model comprises a plurality of binomial distributions of sequencing reads comprising a locus corresponding to variant motif m, with each binomial distribution having a probability of a sequencing read being an alternate read equal to an estimated fraction selected from the plurality of estimated fractions.

23. The method of any one of embodiments 1-18, wherein determining the fraction for the individual comprises:
determining, for each variant motif-specific model, a statistical value indicative of a likelihood of each of a plurality of estimated fractions, given the sequencing data for the nucleic acid molecules obtained from the fluidic sample from the individual corresponding to the respective variant motif and control sequencing data for nucleic acid molecules obtained from one or more control fluidic samples corresponding to the respective variant motif, wherein the control sequencing data is adjusted for one or more non-zero estimated fractions; and
determining a most likely fraction given the statistical values for each variant motif.

24. The method of embodiment 23, wherein the control sequencing data is adjusted for each of the one or more non-zero estimated fractions using a random realization method with a distribution probability equal to the respective non-zero estimated fraction.

25. The method of embodiment 24, wherein, for each of a non-zero estimated tumor fraction, the statistical value indicative of the likelihood is an average of a plurality of likelihood values obtained using a plurality of random realizations, each with a distribution probability equal to the respective non-zero estimated fraction.

26. The method of any one of embodiments 23-25, wherein the one or more control fluidic samples comprises a plurality of control fluidic samples.

27. The method of any one of embodiments 21-26, wherein each statistical value is determined using an exact test.

28. The method of embodiment 27, wherein each statistical value is determined using Fisher's exact test.

29. The method of any one of embodiments 1-28, further comprising determining whether the difference between the fraction for the individual is greater than a background level with statistical significance.

30. The method of any one of embodiments 1-29, wherein the fraction for the individual is a tumor fraction.

31. The method of any one of embodiments 1-30, wherein the nucleic acid molecules are cell-free DNA (cfDNA) molecules.

32. The method of any one of embodiments 1-31, further comprising generating the personalized disease-associated SNV panel.

33. The method of embodiment 32, wherein the personalized disease-associated SNV panel comprises SNVs detected from sequencing data for nucleic acid molecules derived from a diseased tissue sample.

34. The method of embodiment 33, wherein the sample of the diseased tissue is a tumor biopsy sample obtained from the individual.

35. The method of any one of embodiments 1-34, further comprising excluding, from the personalized disease-associated SNV panel, SNVs other than single nucleotide polymorphisms (SNPs).

36. The method of any one of embodiments 1-35, further comprising excluding, from the personalized disease-associated SNV panel, SNVs present in a general population of individuals at an allele frequency greater than a predetermined allele threshold.

37. The method of embodiment 36, wherein the predetermined allele threshold is about 0.01.

38. The method of any one of embodiments 1-37, further comprising excluding, from the personalized disease-associated SNV panel, SNVs at loci with two or more non-reference alleles.

39. The method of any one of embodiments 1-38, further comprising excluding, from the personalized disease-associated SNV panel, SNVs within a low complexity region.

40. The method of any one of embodiments 1-39, further comprising excluding, from the personalized disease-associate SNV panel, SNVs characterized as likely germline variants or likely non-disease related somatic variants.

41. The method of embodiment 40, wherein the SNVs characterized as likely germline variants or as likely non-disease related somatic variants are characterized by sequencing nucleic acid molecules derived from a sample of non-diseased tissue obtained from the individual.

42. The method of any one of embodiments 1-41, wherein nucleic acid molecules derived from a sample of non-diseased tissue obtained from the individual are sequenced to obtain non-diseased tissue sequencing data, and the method further comprises excluding, from the personalized disease-associate SNV panel, SNVs at loci that have no sequencing coverage within the non-diseased tissue sequencing data.

43. The method of embodiment 41 or 42, wherein the sample of non-diseased tissue comprises white blood cells.

44. The method of embodiment 41 or 42, wherein the sample of non-diseased tissue comprises peripheral blood mononuclear cells.

45. The method of any one of embodiments 41-44, wherein the sample of non-diseased tissue is a buffy coat.

46. The method of any one of embodiments 1-45, further comprising excluding, from the personalized disease-associate SNV panel, SNVs at loci associated with a predetermined number or proportion of sequencing reads that have a mapping quality score below a predetermined mapping quality threshold.

47. The method of any one of embodiments 1-46, further comprising excluding, from the personalized disease-associate SNV panel, SNVs at loci that have a bias for reference reads or alternate reads.

48. The method of any one of embodiments 1-47, wherein nucleic acid molecules derived from a diseased tissue sample obtained from the individual are sequenced to obtain diseased tissue sequencing data, and the method further comprises excluding, from the personalized disease-associate SNV panel, SNVs that have a variant allele fraction in the nucleic acid molecules derived from the diseased tissue sample lower than a predetermined low-fraction threshold.

49. The method of any one of embodiments 1-48, wherein nucleic acid molecules derived from a diseased tissue sample obtained from the individual are sequenced to obtain diseased tissue sequencing data, and the method further comprises excluding, from the personalized disease-associate SNV panel, SNVs that have a variant allele fraction in the nucleic acid molecules derived from the diseased tissue sample higher than a predetermined high-fraction threshold.

50. The method of any one of embodiments 1-49, further comprising identifying one or more outlier SNVs within the personalized disease-associated SNV panel that are associated with a locus-specific fraction outlier, given the sequencing data for nucleic acid molecules obtained from a fluidic sample from the individual, and excluding sequencing data associated with said one or more outlier SNVs from the plurality of variant motif-specific models.

51. The method of any one of embodiments 1-50, wherein the method further comprises measuring a recurrence of the disease.

52. The method of any one of embodiments 1-51, wherein the method further comprises measuring a progression or regression of the disease by comparing the level of the disease to a previously measured level of the disease.

53. The method of embodiment 52, wherein progression or regression of the disease is based on a statistically significant change in the measured level of the disease compared to the previously measured level of the disease.

54. The method of any one of embodiments 1-53, wherein the fluidic sample is a blood sample, a plasma sample, a saliva sample, a urine sample, or a fecal sample.

55. The method of any one of embodiments 1-54, wherein the disease is cancer.

56. The method of embodiment 55, wherein the cancer is a metastatic cancer.

57. The method of any one of embodiments 1-56, wherein the sequencing data is untargeted sequencing data.

58. The method of embodiment 57, wherein the sequencing data is obtained from an untargeted whole genome.

59. The method of any one of embodiments 1-58, wherein the mean sequencing depth of the sequencing data is at least 0.01.

60. The method of any one of embodiments 1-59, wherein the mean sequencing depth of the sequencing data is less than about 100.

61. The method of any one of embodiments 1-60, wherein the mean sequencing depth of the sequencing data is less than about 10.

62. The method of any one of embodiments 1-61, wherein the mean sequencing depth of the sequencing data is less than about 1.

63. The method of any one of embodiments 1-62, wherein the personalized disease-associated SNV panel comprises passenger mutations.

64. The method of any one of embodiments 1-63, wherein the personalized disease-associated SNV panel comprises driver mutations.

65. The method of any one of embodiments 1-64, wherein the selected loci from the personalized disease-associated SNV panel comprise about 300 or more SNV loci.

66. The method of any one of embodiments 1-65, wherein the sequencing data is obtained using surface-based sequencing of nucleic acid molecules, and wherein the nucleic acid molecules are not amplified prior to attaching the nucleic acid molecules to a surface.

67. The method of any one of embodiments 1-66, wherein the sequencing data is obtained without using unique molecular identifiers (UMIs).

68. The method of any one of embodiments 1-67, wherein the sequencing data is obtained without using sample identification barcodes.

69. The method of any one of embodiments 1-68, wherein the background factor is based on sequencing data for nucleic acid molecules obtained from a plurality of control individuals.

70. The method of embodiment 69, wherein the sequencing data for nucleic acid molecules obtained from a plurality of control individuals comprises sequencing reads associated with loci selected from the personalized disease-associated SNV panel.

71. The method of embodiment 69 or 70, wherein the sequencing data for the nucleic acid molecules of the individual and the sequencing data for the nucleic acid molecules for the plurality of control individuals are simultaneously obtained in a pooled sample.

72. The method of any one of embodiments 1-71, further comprising generating a report that indicates the presence, absence, or level of disease in the individual.

73. The method of embodiment 72, further comprising providing the report to a patient or a healthcare representative of the patient.

74. A system, comprising:
one or more processors; and
a non-transitory computer-readable storage medium that stores one or more programs comprising instructions for implementing the method of any one of embodiments 1-73.

75. A system, comprising:
one or more processors; and
a non-transitory computer-readable storage medium that stores one or more programs comprising instructions that, when executed by the one or more processors, determines a level of a disease in an individual according to a method comprising:
   receiving, at the one or more processors, sequencing data for nucleic acid molecules obtained from a fluidic sample from the individual, the sequencing data comprising sequencing reads associated with loci selected from a personalized disease-associated small nucleotide variant (SNV) panel;
   generating, using the one or more processors and the sequencing data, a plurality of variant motif-specific models that each associate sequencing data corresponding to a respective variant motif, a background factor indicative of a false positive error rate for the respective variant motif, and an estimated fraction of the nucleic acid molecules associated with the disease; and
   determining from the plurality of variant motif-specific models, using the one or more processors, a fraction of the nucleic acid molecules associated with the disease for the individual, wherein the fraction indicates the level of the disease in the individual.

76. The system of embodiment 75, wherein the level of disease in the individual is a presence or absence of the disease.

77. The system of embodiment 75, wherein the level of disease in the individual is a quantitative value indicating the severity of the disease.

78. A system, comprising:
one or more processors; and
a non-transitory computer-readable storage medium that stores one or more programs comprising instructions that, when executed by the one or more processors, determines a presence or absence of a disease in an individual according to a method comprising:
   receiving, at the one or more processors, sequencing data for nucleic acid molecules obtained from a fluidic sample from the individual, the sequencing data comprising sequencing reads associated with loci selected from a personalized disease-associated small nucleotide variant (SNV) panel;
   generating, using the one or more processors and the sequencing data, a plurality of variant motif-specific models that each associate sequencing data corresponding to a respective variant motif, a background factor indicative of a false positive error rate for the respective variant motif, and an estimated fraction of the nucleic acid molecules associated with the disease; and
   determining from the plurality of variant motif-specific models, using the one or more processors, a fraction of the nucleic acid molecules associated with the disease for the individual; and
   comparing, using the one or more processors, the fraction to a background level, wherein the fraction being above the background level indicates the presence of the disease in the individual.

79. The system of any one of embodiments 75-78, wherein the sequencing data is generated by sequencing the nucleic acid molecules using non-terminating nucleotides provided in separate nucleotide flows according to a flow-cycle order comprising a plurality of flow positions, wherein the flow positions correspond to the nucleotide flows.

80. The system of any one of embodiments 75-79, further comprising a sequencer, wherein the method further comprises generating the sequencing data using the sequencer.

81. The system of embodiment 80, wherein generating the sequencing data comprises sequencing the nucleic acid molecules using non-terminating nucleotides provided in separate nucleotide flows according to a flow-cycle order comprising a plurality of flow positions, wherein the flow positions correspond to the nucleotide flows.

82. The system of any one of embodiments 75-81, wherein the method further comprises:
for each of the sequencing reads, determining, using the one or more processors, a likelihood that the sequencing read corresponds to a variant sequence and a likelihood that the sequencing read corresponds to a reference sequence, and
for a respective sequence read, if the difference between the likelihood that the respective sequencing read corresponds to the variant sequence and the likelihood that the respective sequencing read corresponds to a reference sequence is less than a predetermined likelihood difference threshold, then excluding, using the one or more processors, sequencing data corresponding to the respective sequencing read from the plurality of variant motif-specific models.

83. The system of embodiment 82, wherein the variant sequence and the reference sequence are corresponding haplotype sequences.

84. The system of embodiment 82 or 83, wherein the variant sequence and the reference sequence differ by at least two bases.

85. The system of embodiment 82 or 83, wherein the variant sequence and the reference sequence comprise at least to loci from the personalized disease-associated SNV panel.

86. The system of any one of embodiments 75-81, wherein the method further comprises, for each of the sequencing reads:
identifying, using the one or more processors, a variant locus from the personalized disease-associated SNV panel within the sequencing read, wherein the variant locus is associated with a single base variant;
trimming, using the one or more processors, the sequencing read to generate a trimmed sequencing read comprising the variant locus and excluding any other variant locus from the personalized disease-associated SNV panel;
determining, using the one or more processors, a likelihood that the trimmed sequencing read corresponds to a variant sequence comprising and a likelihood that the sequencing read corresponds to a reference sequence, wherein the variant sequence and the reference sequence each comprises the variant locus and excludes any other variant locus from the personalized disease-associated SNV panel; and
for a respective trimmed sequencing read, if the difference between the likelihood that the respective trimmed sequencing read corresponds to the variant sequence and the likelihood that the respective trimmed sequencing read corresponds to a reference sequence is less than a predetermined likelihood difference threshold, then excluding, using the one or more processors, sequencing data corresponding to the respective trimmed sequencing read from the plurality of variant motif-specific models.

87. The system of any one of embodiments 82-86, wherein the predetermined likelihood difference threshold is set at a value of 5 orders of magnitude or higher.

88. The system of any one of embodiments 75-87, wherein at least 90% of SNVs in the personalized disease-associated SNV panel are associated with SNV sequencing data that differs from reference sequencing data associated with a reference sequence at two or more flow positions, wherein the SNV sequencing data and the reference sequencing data are sequenced using non-terminating nucleotides provided in separate nucleotide flows according to a flow-cycle order comprising a plurality of flow positions, wherein the flow positions correspond to nucleotide flows.

89. The system of any one of embodiments 75-88, wherein at least 90% of SNVs in the personalized disease-associated SNV panel are associated with SNV sequencing data that differs from reference sequencing data associated with a reference sequence across one or more flow cycles in a flow-cycle order, wherein the sequencing data and the reference sequencing data are sequenced using non-terminating nucleotides provided in separate nucleotide flows according to the flow-cycle order.

90. The system of any one of embodiments 75-89, wherein the method further comprises characterizing, using the one or more processors, the sequencing reads as an alternate read, a reference read, or an ambiguous read, wherein a sequencing read characterized as an ambiguous read is excluded from the plurality of variant motif-specific models.

91. The system of any one of embodiments 75-90, wherein the plurality of variant motif-specific models comprises a respective variant motif-specific model for each of a plurality of trinucleotide SNP motifs.

92. The system of embodiment 91, wherein the plurality of variant motif-specific models comprises 192 trinucleotide SNP variant motif-specific models.

93. The system of any one of embodiments 75-92, wherein each variant motif-specific model associates the sequencing data corresponding to variant motif, m, to the background factor, BGₘ, and the estimated faction, F, according to:

Nₘ^{alt}=(F+BGₘ)Nₘ^{total},

wherein Nₘ^{alt} is a number of alternative sequencing reads comprising a locus corresponding to variant motif m and Nₘ^{total} is a total number of sequencing reads comprising a locus corresponding to variant motif m.

94. The system of any one of embodiments 75-93, wherein each variant motif-specific model is a binomial distribution of the sequencing reads comprising a locus corresponding to variant motif m, with a probability, pₘ, of observing an alternative sequencing read comprising a locus corresponding to variant motif m based on pₘ=F+BGₘ, wherein F is the estimated fraction, and BGₘ is the background factor.

95. The system of any one of embodiments 75-94, wherein determining the fraction for the individual comprises determining a maximum likelihood estimate for the fraction given the plurality of variant motif-specific models.

96. The system of any one of embodiments 75-95, wherein determining the fraction for the individual comprises:
determining, using the one or more processors, for each variant motif-specific model, a statistical value indicative of a likelihood of each of a plurality of estimated fractions, given the sequencing data for the nucleic acid molecules obtained from the fluidic sample from the individual corresponding to the respective variant motif, and
determining, using the one or more processors, a most likely fraction given the statistical values for each variant motif.

97. The system of embodiment 96, wherein each variant motif-specific model comprises a plurality of binomial distributions of sequencing reads comprising a locus corresponding to variant motif m, with each binomial distribution having a probability of a sequencing read being an alternate read equal to an estimated fraction selected from the plurality of estimated fractions.

98. The system of any one of embodiments 75-95, wherein determining the fraction for the individual comprises:
determining for each variant motif-specific model, using the one or more processors, a statistical value indicative of a likelihood of each of a plurality of estimated fractions, given the sequencing data for the nucleic acid molecules obtained from the fluidic sample from the individual corresponding to the respective variant motif and control sequencing data for nucleic acid molecules obtained from one or more control fluidic samples corresponding to the respective variant motif, wherein the control sequencing data is adjusted for one or more non-zero estimated fractions; and
determining, using the one or more processors, a most likely fraction given the statistical values for each variant motif.

99. The system of embodiment 98, wherein the control sequencing data is adjusted for each of the one or more non-zero estimated fractions using a random realization method with a distribution probability equal to the respective non-zero estimated fraction.

100. The system of embodiment 99, wherein, for each of a non-zero estimated tumor fraction, the statistical value indicative of the likelihood is an average of a plurality of likelihood values obtained using a plurality of random realizations, each with a distribution probability equal to the respective non-zero estimated fraction.

101. The system of any one of embodiments 98-100, wherein the one or more control fluidic samples comprises a plurality of control fluidic samples.

102. The system of any one of embodiments 96-101, wherein each statistical value is determined using an exact test.

103. The system of embodiment 102, wherein each statistical value is determined using Fisher's exact test.

104. The system of any one of embodiments 75-103, wherein the method further comprises determining, using the one or more processors, whether the difference between the fraction for the individual is greater than a background level with statistical significance.

105. The system of any one of embodiments 75-104, wherein the fraction for the individual is a tumor fraction.

106. The system of any one of embodiments 75-105, wherein the nucleic acid molecules are cell-free DNA (cfDNA) molecules.

107. The system of any one of embodiments 75-106, wherein the method further comprises generating, using the one or more processors, the personalized disease-associated SNV panel.

108. The system of embodiment 107, wherein the personalized disease-associated SNV panel comprises SNVs detected from sequencing data for nucleic acid molecules derived from a diseased tissue sample.

109. The system of embodiment 108, wherein the sample of the diseased tissue is a tumor biopsy sample obtained from the individual.

110. The system of any one of embodiments 75-109, wherein the method further comprises excluding from the personalized disease-associated SNV panel, using the one or more processors, SNVs other than single nucleotide polymorphisms (SNPs).

111. The system of any one of embodiments 75-110, wherein the method further comprises excluding from the personalized disease-associated SNV panel, using the one or more processors, SNVs present in a general population of individuals at an allele frequency greater than a predetermined allele threshold.

112. The system of embodiment 111, wherein the predetermined allele threshold is about 0.01.

113. The system of any one of embodiments 75-112, wherein the method further comprises excluding from the personalized disease-associated SNV panel, using the one or more processors, SNVs at loci with two or more non-reference alleles.

114. The system of any one of embodiments 75-113, wherein the method further comprises excluding from the personalized disease-associated SNV panel, using the one or more processors, SNVs within a low complexity region.

115. The system of any one of embodiments 75-114 wherein the method further comprises excluding from the personalized disease-associate SNV panel, using the one or more processors, SNVs characterized as likely germline variants or likely non-disease related somatic variants.

116. The system of embodiment 115, wherein the SNVs characterized as likely germline variants or as likely non-disease related somatic variants are characterized by sequencing nucleic acid molecules derived from a sample of non-diseased tissue obtained from the individual.

117. The system of any one of embodiments 75-116, wherein nucleic acid molecules derived from a sample of non-diseased tissue obtained from the individual are sequenced to obtain non-diseased tissue sequencing data, and the method further comprises excluding from the personalized disease-associate SNV panel, using the one or more processors, SNVs at loci that have no sequencing coverage within the non-diseased tissue sequencing data.

118. The system of embodiment 116 or 117, wherein the sample of non-diseased tissue comprises white blood cells.

119. The system of embodiment 116 or 117, wherein the sample of non-diseased tissue comprises peripheral blood mononuclear cells.

120. The system of any one of embodiments 116-119, wherein the sample of non-diseased tissue is a buffy coat.

121. The system of any one of embodiments 75-120, wherein the method further comprises excluding from the personalized disease-associate SNV panel, using the one or more processors, SNVs at loci associated with a predetermined number or proportion of sequencing reads that have a mapping quality score below a predetermined mapping quality threshold.

122. The system of any one of embodiments 75-121, wherein the method further comprises excluding from the personalized disease-associate SNV panel, using one or more processors, SNVs at loci that have a bias for reference reads or alternate reads.

123. The system of any one of embodiments 75-122, wherein nucleic acid molecules derived from a diseased tissue sample obtained from the individual are sequenced to obtain diseased tissue sequencing data, and the method further comprises excluding from the personalized disease-associate SNV panel, using the one or more processors, SNVs that have a variant allele fraction in the nucleic acid molecules derived from the diseased tissue sample lower than a predetermined low-fraction threshold.

124. The system of any one of embodiments 75-123, wherein nucleic acid molecules derived from a diseased tissue sample obtained from the individual are sequenced to obtain diseased tissue sequencing data, and the method further comprises excluding from the personalized disease-associate SNV panel, using the one or more processors, SNVs that have a variant allele fraction in the nucleic acid molecules derived from the diseased tissue sample higher than a predetermined high-fraction threshold.

125. The system of any one of embodiments 75-124, wherein the method further comprises identifying, using the one or more processors, one or more outlier SNVs within the personalized disease-associated SNV panel that are associated with a locus-specific fraction outlier, given the sequencing data for nucleic acid molecules obtained from a fluidic sample from the individual, and excluding, using the one or more processors, sequencing data associated with said one or more outlier SNVs from the plurality of variant motif-specific models.

126. The system of any one of embodiments 75-125, wherein the method further comprises determining, using the one or more processors, a recurrence of the disease.

127. The system of any one of embodiments 75-126, wherein the method further comprises determining a progression or regression of the disease by comparing, using the one or more processors, the level of the disease to a previously measured level of the disease.

128. The system of embodiment 127, wherein progression or regression of the disease is based on a statistically significant change in the measured level of the disease compared to the previously measured level of the disease.

129. The system of any one of embodiments 75-128, wherein the fluidic sample is a blood sample, a plasma sample, a saliva sample, a urine sample, or a fecal sample.

130. The system of any one of embodiments 75-129, wherein the disease is cancer.

131. The system of embodiment 130, wherein the cancer is a metastatic cancer.

132. The system of any one of embodiments 75-131, wherein the sequencing data is untargeted sequencing data.

133. The system of embodiment 132, wherein the sequencing data is obtained from an untargeted whole genome.

134. The system of any one of embodiments 75-133, wherein the mean sequencing depth of the sequencing data is at least 0.01.

135. The system of any one of embodiments 75-134, wherein the mean sequencing depth of the sequencing data is less than about 100.

136. The system of any one of embodiments 75-135, wherein the mean sequencing depth of the sequencing data is less than about 10.

137. The system of any one of embodiments 75-136, wherein the mean sequencing depth of the sequencing data is less than about 1.

138. The system of any one of embodiments 75-137, wherein the personalized disease-associated SNV panel comprises passenger mutations.

139. The system of any one of embodiments 75-138, wherein the personalized disease-associated SNV panel comprises driver mutations.

140. The system of any one of embodiments 75-139, wherein the selected loci from the personalized disease-associated SNV panel comprise about 300 or more SNV loci.

141. The system of any one of embodiments 75-140, wherein the sequencing data is obtained using surface-based sequencing of nucleic acid molecules, and wherein the nucleic acid molecules are not amplified prior to attaching the nucleic acid molecules to a surface.

142. The system of any one of embodiments 75-141, wherein the sequencing data is obtained without using unique molecular identifiers (UMIs).

143. The system of any one of embodiments 75-142, wherein the sequencing data is obtained without using sample identification barcodes.

144. The system of any one of embodiments 75-143, wherein the background factor is based on sequencing data for nucleic acid molecules obtained from a plurality of control individuals.

145. The system of embodiment 144, wherein the sequencing data for nucleic acid molecules obtained from a plurality of control individuals comprises sequencing reads associated with loci selected from the personalized disease-associated SNV panel.

146. The system of embodiment 144 or 145, wherein the sequencing data for the nucleic acid molecules of the individual and the sequencing data for the nucleic acid molecules for the plurality of control individuals are simultaneously obtained in a pooled sample.

147. The system of any one of embodiments 75-146, wherein the method further comprises generating, using the one or more processors, a report that indicates the presence, absence, or level of disease in the individual.

148. The system of embodiment 147, wherein the method further comprises transmitting, using a computer network link, the report to a patient or a healthcare representative of the patient.

149. A non-transitory computer-readable storage medium that stores one or more programs comprising instructions that, when executed by one or more processors, determines a level of a disease in an individual according to a method comprising:
receiving, at the one or more processors, sequencing data for nucleic acid molecules obtained from a fluidic sample from the individual, the sequencing data comprising sequencing reads associated with loci selected from a personalized disease-associated small nucleotide variant (SNV) panel;
generating, using the one or more processors and the sequencing data, a plurality of variant motif-specific models that each associate sequencing data corresponding to a respective variant motif, a background factor indicative of a false positive error rate for the respective variant motif, and an estimated fraction of the nucleic acid molecules associated with the disease; and
determining from the plurality of variant motif-specific models, using the one or more processors, a fraction of the nucleic acid molecules associated with the disease for the individual, wherein the fraction indicates the level of the disease in the individual.

150. The non-transitory computer-readable storage medium of embodiment 149, wherein the level of disease in the individual is a presence or absence of the disease.

151. The non-transitory computer-readable storage medium of embodiment 149, wherein the level of disease in the individual is a quantitative value indicating the severity of the disease.

152. A non-transitory computer-readable storage medium that stores one or more programs comprising instructions that, when executed by one or more processors, determines a presence or absence of a disease in an individual according to a method comprising:
receiving, at the one or more processors, sequencing data for nucleic acid molecules obtained from a fluidic sample from the individual, the sequencing data comprising sequencing reads associated with loci selected from a personalized disease-associated small nucleotide variant (SNV) panel;
generating, using the one or more processors and the sequencing data, a plurality of variant motif-specific models that each associate sequencing data corresponding to a respective variant motif, a background factor indicative of a false positive error rate for the respective variant motif, and an estimated fraction of the nucleic acid molecules associated with the disease; and
determining from the plurality of variant motif-specific models, using the one or more processors, a fraction of the nucleic acid molecules associated with the disease for the individual; and
comparing, using the one or more processors, the fraction to a background level, wherein the fraction being above the background level indicates the presence of the disease in the individual.

153. The non-transitory computer-readable storage medium of any one of embodiments 149-152, wherein the sequencing data is generated by sequencing the nucleic acid molecules using non-terminating nucleotides provided in separate nucleotide flows according to a flow-cycle order comprising a plurality of flow positions, wherein the flow positions correspond to the nucleotide flows.

154. The non-transitory computer-readable storage medium of any one of embodiments 149-152, further comprising instructions that, when executed by one or more processors, operate a sequencer to generate the sequencing data.

155. The non-transitory computer-readable storage medium of embodiment 154, wherein the sequencer is operated to sequence the nucleic acid molecules using non-terminating nucleotides provided in separate nucleotide flows according to a flow-cycle order comprising a plurality of flow positions, wherein the flow positions correspond to the nucleotide flows.

156. The non-transitory computer-readable storage medium of any one of embodiments 149-155, wherein the method further comprises:
for each of the sequencing reads, determining, using the one or more processors, a likelihood that the sequencing read corresponds to a variant sequence and a likelihood that the sequencing read corresponds to a reference sequence, and
for a respective sequence read, if the difference between the likelihood that the respective sequencing read corresponds to the variant sequence and the likelihood that the respective sequencing read corresponds to a reference sequence is less than a predetermined likelihood difference threshold, then excluding, using the one or more processors, sequencing data corresponding to the respective sequencing read from the plurality of variant motif-specific models.

157. The non-transitory computer-readable storage medium of embodiment 156, wherein the variant sequence and the reference sequence are corresponding haplotype sequences.

158. The non-transitory computer-readable storage medium of embodiment 156 or 157, wherein the variant sequence and the reference sequence differ by at least two bases.

159. The non-transitory computer-readable storage medium of embodiment 156 or 157, wherein the variant sequence and the reference sequence comprise at least to loci from the personalized disease-associated SNV panel.

160. The non-transitory computer-readable storage medium of any one of embodiments 149-155, wherein the method further comprises, for each of the sequencing reads:
identifying, using the one or more processors, a variant locus from the personalized disease-associated SNV panel within the sequencing read, wherein the variant locus is associated with a single base variant;
trimming, using the one or more processors, the sequencing read to generate a trimmed sequencing read comprising the variant locus and excluding any other variant locus from the personalized disease-associated SNV panel;
determining, using the one or more processors, a likelihood that the trimmed sequencing read corresponds to a variant sequence comprising and a likelihood that the sequencing read corresponds to a reference sequence, wherein the variant sequence and the reference sequence each comprises the variant locus and excludes any other variant locus from the personalized disease-associated SNV panel; and
for a respective trimmed sequencing read, if the difference between the likelihood that the respective trimmed sequencing read corresponds to the variant sequence and the likelihood that the respective trimmed sequencing read corresponds to a reference sequence is less than a predetermined likelihood difference threshold, then excluding, using the one or more processors, sequencing data corresponding to the respective trimmed sequencing read from the plurality of variant motif-specific models.

161. The non-transitory computer-readable storage medium of any one of embodiments 156-160, wherein the predetermined likelihood difference threshold is set at a value of 5 orders of magnitude or higher.

162. The non-transitory computer-readable storage medium of any one of embodiments 149-161, wherein at least 90% of SNVs in the personalized disease-associated SNV panel are associated with SNV sequencing data that differs from reference sequencing data associated with a reference sequence at two or more flow positions, wherein the SNV sequencing data and the reference sequencing data are sequenced using non-terminating nucleotides provided in separate nucleotide flows according to a flow-cycle order comprising a plurality of flow positions, wherein the flow positions correspond to nucleotide flows.

163. The non-transitory computer-readable storage medium of any one of embodiments 149-162, wherein at least 90% of SNVs in the personalized disease-associated SNV panel are associated with SNV sequencing data that differs from reference sequencing data associated with a reference sequence across one or more flow cycles in a flow-cycle order, wherein the sequencing data and the reference sequencing data are sequenced using non-terminating nucleotides provided in separate nucleotide flows according to the flow-cycle order.

164. The non-transitory computer-readable storage medium of any one of embodiments 149-163, wherein the method further comprises characterizing, using the one or more processors, the sequencing reads as an alternate read, a reference read, or an ambiguous read, wherein a sequencing read characterized as an ambiguous read is excluded from the plurality of variant motif-specific models.

165. The non-transitory computer-readable storage medium of any one of embodiments 149-164, wherein the plurality of variant motif-specific models comprises a respective variant motif-specific model for each of a plurality of trinucleotide SNP motifs.

166. The non-transitory computer-readable storage medium of embodiment 166, wherein the plurality of variant motif-specific models comprises 192 trinucleotide SNP variant motif-specific models.

167. The non-transitory computer-readable storage medium of any one of embodiments 149-166, wherein each variant motif-specific model associates the sequencing data corresponding to variant motif, m, to the background factor, BGₘ, and the estimated faction, F, according to:

Nₘ^{alt}=(F+BGₘ)Nₘ^{total},

wherein Nₘ^{alt} is a number of alternative sequencing reads comprising a locus corresponding to variant motif m and Nₘ^{total} is a total number of sequencing reads comprising a locus corresponding to variant motif m.

168. The non-transitory computer-readable storage medium of any one of embodiments 149-167, wherein each variant motif-specific model is a binomial distribution of the sequencing reads comprising a locus corresponding to variant motif m, with a probability, pₘ, of observing an alternative sequencing read comprising a locus corresponding to variant motif m based on pₘ=F+BGₘ, wherein F is the estimated fraction, and BGₘ is the background factor.

169. The non-transitory computer-readable storage medium of any one of embodiments 149-168, wherein determining the fraction for the individual comprises determining a maximum likelihood estimate for the fraction given the plurality of variant motif-specific models.

170. The non-transitory computer-readable storage medium of any one of embodiments 149-169, wherein determining the fraction for the individual comprises:
determining, using the one or more processors, for each variant motif-specific model, a statistical value indicative of a likelihood of each of a plurality of estimated fractions, given the sequencing data for the nucleic acid molecules obtained from the fluidic sample from the individual corresponding to the respective variant motif, and
determining, using the one or more processors, a most likely fraction given the statistical values for each variant motif.

171. The non-transitory computer-readable storage medium of embodiment 170, wherein each variant motif-specific model comprises a plurality of binomial distributions of sequencing reads comprising a locus corresponding to variant motif m, with each binomial distribution having a probability of a sequencing read being an alternate read equal to an estimated fraction selected from the plurality of estimated fractions.

172. The non-transitory computer-readable storage medium of any one of embodiments 149-170, wherein determining the fraction for the individual comprises:
determining for each variant motif-specific model, using the one or more processors, a statistical value indicative of a likelihood of each of a plurality of estimated fractions, given the sequencing data for the nucleic acid molecules obtained from the fluidic sample from the individual corresponding to the respective variant motif and control sequencing data for nucleic acid molecules obtained from one or more control fluidic samples corresponding to the respective variant motif, wherein the control sequencing data is adjusted for one or more non-zero estimated fractions; and
determining, using the one or more processors, a most likely fraction given the statistical values for each variant motif.

173. The non-transitory computer-readable storage medium of embodiment 172, wherein the control sequencing data is adjusted for each of the one or more non-zero estimated fractions using a random realization method with a distribution probability equal to the respective non-zero estimated fraction.

174. The non-transitory computer-readable storage medium of embodiment 173, wherein, for each of a non-zero estimated tumor fraction, the statistical value indicative of the likelihood is an average of a plurality of likelihood values obtained using a plurality of random realizations, each with a distribution probability equal to the respective non-zero estimated fraction.

175. The non-transitory computer-readable storage medium of any one of embodiments 172-174, wherein the one or more control fluidic samples comprises a plurality of control fluidic samples.

176. The non-transitory computer-readable storage medium of any one of embodiments 170-175, wherein each statistical value is determined using an exact test.

177. The non-transitory computer-readable storage medium of embodiment 176, wherein each statistical value is determined using Fisher's exact test.

178. The non-transitory computer-readable storage medium of any one of embodiments 149-177, wherein the method further comprises determining, using the one or more processors, whether the difference between the fraction for the individual is greater than a background level with statistical significance.

179. The non-transitory computer-readable storage medium of any one of embodiments 149-178, wherein the fraction for the individual is a tumor fraction.

180. The non-transitory computer-readable storage medium of any one of embodiments 149-179, wherein the nucleic acid molecules are cell-free DNA (cfDNA) molecules.

181. The non-transitory computer-readable storage medium of any one of embodiments 149-180, wherein the method further comprises generating, using the one or more processors, the personalized disease-associated SNV panel.

182. The non-transitory computer-readable storage medium of embodiment 181, wherein the personalized disease-associated SNV panel comprises SNVs detected from sequencing data for nucleic acid molecules derived from a diseased tissue sample.

183. The non-transitory computer-readable storage medium of embodiment 182, wherein the sample of the diseased tissue is a tumor biopsy sample obtained from the individual.

184. The non-transitory computer-readable storage medium of any one of embodiments 149-183, wherein the method further comprises excluding from the personalized disease-associated SNV panel, using the one or more processors, SNVs other than single nucleotide polymorphisms (SNPs).

185. The non-transitory computer-readable storage medium of any one of embodiments 149-184, wherein the method further comprises excluding from the personalized disease-associated SNV panel, using the one or more processors, SNVs present in a general population of individuals at an allele frequency greater than a predetermined allele threshold.

186. The non-transitory computer-readable storage medium of embodiment 185, wherein the predetermined allele threshold is about 0.01.

187. The non-transitory computer-readable storage medium of any one of embodiments 149-186, wherein the method further comprises excluding from the personalized disease-associated SNV panel, using the one or more processors, SNVs at loci with two or more non-reference alleles.

188. The non-transitory computer-readable storage medium of any one of embodiments 149-187, wherein the method further comprises excluding from the personalized disease-associated SNV panel, using the one or more processors, SNVs within a low complexity region.

189. The non-transitory computer-readable storage medium of any one of embodiments 149-188, wherein the method further comprises excluding from the personalized disease-associate SNV panel, using the one or more processors, SNVs characterized as likely germline variants or likely non-disease related somatic variants.

190. The non-transitory computer-readable storage medium of embodiment 189, wherein the SNVs characterized as likely germline variants or as likely non-disease related somatic variants are characterized by sequencing nucleic acid molecules derived from a sample of non-diseased tissue obtained from the individual.

191. The non-transitory computer-readable storage medium of any one of embodiments 149-190, wherein nucleic acid molecules derived from a sample of non-diseased tissue obtained from the individual are sequenced to obtain non-diseased tissue sequencing data, and the method further comprises excluding from the personalized disease-associate SNV panel, using the one or more processors, SNVs at loci that have no sequencing coverage within the non-diseased tissue sequencing data.

192. The non-transitory computer-readable storage medium of embodiment 190 or 191, wherein the sample of non-diseased tissue comprises white blood cells.

193. The non-transitory computer-readable storage medium of embodiment 190 or 191, wherein the sample of non-diseased tissue comprises peripheral blood mononuclear cells.

194. The non-transitory computer-readable storage medium of any one of embodiments 190-193, wherein the sample of non-diseased tissue is a buffy coat.

195. The non-transitory computer-readable storage medium of any one of embodiments 149-194, wherein the method further comprises excluding from the personalized disease-associate SNV panel, using the one or more processors, SNVs at loci associated with a predetermined number or proportion of sequencing reads that have a mapping quality score below a predetermined mapping quality threshold.

196. The non-transitory computer-readable storage medium of any one of embodiments 149-195, wherein the method further comprises excluding from the personalized disease-associate SNV panel, using one or more processors, SNVs at loci that have a bias for reference reads or alternate reads.

197. The non-transitory computer-readable storage medium of any one of embodiments 149-196, wherein nucleic acid molecules derived from a diseased tissue sample obtained from the individual are sequenced to obtain diseased tissue sequencing data, and the method further comprises excluding from the personalized disease-associate SNV panel, using the one or more processors, SNVs that have a variant allele fraction in the nucleic acid molecules derived from the diseased tissue sample lower than a predetermined low-fraction threshold.

198. The non-transitory computer-readable storage medium of any one of embodiments 149-197, wherein nucleic acid molecules derived from a diseased tissue sample obtained from the individual are sequenced to obtain diseased tissue sequencing data, and the method further comprises excluding from the personalized disease-associate SNV panel, using the one or more processors, SNVs that have a variant allele fraction in the nucleic acid molecules derived from the diseased tissue sample higher than a predetermined high-fraction threshold.

199. The non-transitory computer-readable storage medium of any one of embodiments 149-198, wherein the method further comprises identifying, using the one or more processors, one or more outlier SNVs within the personalized disease-associated SNV panel that are associated with a locus-specific fraction outlier, given the sequencing data for nucleic acid molecules obtained from a fluidic sample from the individual, and excluding, using the one or more processors, sequencing data associated with said one or more outlier SNVs from the plurality of variant motif-specific models.

200. The non-transitory computer-readable storage medium of any one of embodiments 149-199, wherein the method further comprises determining, using the one or more processors, a recurrence of the disease.

201. The non-transitory computer-readable storage medium of any one of embodiments 149-200, wherein the method further comprises determining a progression or regression of the disease by comparing, using the one or more processors, the level of the disease to a previously measured level of the disease.

202. The non-transitory computer-readable storage medium of embodiment 201, wherein progression or regression of the disease is based on a statistically significant change in the measured level of the disease compared to the previously measured level of the disease.

203. The non-transitory computer-readable storage medium of any one of embodiments 149-202, wherein the fluidic sample is a blood sample, a plasma sample, a saliva sample, a urine sample, or a fecal sample.

204. The non-transitory computer-readable storage medium of any one of embodiments 149-203, wherein the disease is cancer.

205. The non-transitory computer-readable storage medium of embodiment 204, wherein the cancer is a metastatic cancer.

206. The non-transitory computer-readable storage medium of any one of embodiments 149-205, wherein the sequencing data is untargeted sequencing data.

207. The non-transitory computer-readable storage medium of embodiment 206, wherein the sequencing data is obtained from an untargeted whole genome.

208. The non-transitory computer-readable storage medium of any one of embodiments 149-207, wherein the mean sequencing depth of the sequencing data is at least 0.01.

209. The non-transitory computer-readable storage medium of any one of embodiments 149-208, wherein the mean sequencing depth of the sequencing data is less than about 100.

210. The non-transitory computer-readable storage medium of any one of embodiments 149-209, wherein the mean sequencing depth of the sequencing data is less than about 10.

211. The non-transitory computer-readable storage medium of any one of embodiments 149-210, wherein the mean sequencing depth of the sequencing data is less than about 1.

212. The non-transitory computer-readable storage medium of any one of embodiments 149-211, wherein the personalized disease-associated SNV panel comprises passenger mutations.

213. The non-transitory computer-readable storage medium of any one of embodiments 149-212, wherein the personalized disease-associated SNV panel comprises driver mutations.

214. The non-transitory computer-readable storage medium of any one of embodiments 149-213, wherein the selected loci from the personalized disease-associated SNV panel comprise about 300 or more SNV loci.

215. The non-transitory computer-readable storage medium of any one of embodiments 149-214, wherein the sequencing data is obtained using surface-based sequencing of nucleic acid molecules, and wherein the nucleic acid molecules are not amplified prior to attaching the nucleic acid molecules to a surface.

216. The non-transitory computer-readable storage medium of any one of embodiments 149-215, wherein the sequencing data is obtained without using unique molecular identifiers (UMIs).

217. The non-transitory computer-readable storage medium of any one of embodiments 149-216, wherein the sequencing data is obtained without using sample identification barcodes.

218. The non-transitory computer-readable storage medium of any one of embodiments 149-217, wherein the background factor is based on sequencing data for nucleic acid molecules obtained from a plurality of control individuals.

219. The non-transitory computer-readable storage medium of embodiment 218, wherein the sequencing data for nucleic acid molecules obtained from a plurality of control individuals comprises sequencing reads associated with loci selected from the personalized disease-associated SNV panel.

220. The non-transitory computer-readable storage medium of embodiment 218 or 219, wherein the sequencing data for the nucleic acid molecules of the individual and the sequencing data for the nucleic acid molecules for the plurality of control individuals are simultaneously obtained in a pooled sample.

221. The non-transitory computer-readable storage medium of any one of embodiments 149-220, wherein the method further comprises generating, using the one or more processors, a report that indicates the presence, absence, or level of disease in the individual.

222. The non-transitory computer-readable storage medium of embodiment 21, wherein the method further comprises transmitting, using a computer network link, the report to a patient or a healthcare representative of the patient.

223. A method, comprising:
generating a sequencing read by sequencing a nucleic acid molecule using non-terminating nucleotides provided in separate nucleotide flows according to a flow-cycle order, wherein the sequencing read comprises a plurality of flow positions that correspond to the nucleotide flows;
identifying, within the sequencing read, a variant locus for a single base variant within a disease-associated small nucleotide variant (SNV) panel;
trimming the sequencing read to generate a trimmed sequencing read comprising the variant locus and excluding any other variant locus from the disease-associated SNV panel;
determining a likelihood that the trimmed sequencing read corresponds to a variant sequence comprising and a likelihood that the sequencing read corresponds to a reference sequence, wherein the variant sequence and the reference sequence each comprises the variant locus and excludes any other variant locus from the disease-associated SNV panel; and
calling the sequencing read, based on the likelihoods, as supporting the presence of a variant at the variant locus, not supporting the presence of a variant at the variant locus, or ambiguous.

224. The method of embodiment 223, wherein the trimmed sequencing read comprises 15 or fewer flow positions.

225. The method of embodiment 223 or 224, wherein the length of the trimmed sequencing read is based on the likelihoods.

226. The method of any one of embodiments 223-225, wherein the disease-associated SNV panel is a personalized disease-associated SNV panel.

227. The method of any one of embodiments 223-226, wherein the variant sequence is associated with a tumor genome and the reference sequence is associated with a non-tumor genome.

228. The method of any one of embodiments 223-227, wherein the method is performed for a plurality of sequencing reads, wherein at least a portion of the sequencing reads in the plurality of sequencing reads comprise different variant loci.

229. The method of embodiment 228, wherein the method further comprises determining a fraction of the nucleic acid molecules associated with the disease for the individual, wherein the fraction indicates the level of the disease in the individual a level of disease in an individual.

230. The method of embodiment 229, wherein the level of disease in the individual is a presence or absence of the disease.

231. The method of embodiment 229, wherein the level of disease in the individual is a quantitative value indicating the severity of the disease.

232. The method of any one of embodiments 229-231, wherein the fraction is a tumor fraction.

233. The method of any one of embodiments 223-232, wherein nucleic acid molecule is obtained from a fluidic sample from an individual.

234. The method of embodiment 233, wherein the fluidic sample is a blood sample, a plasma sample, a saliva sample, a urine sample, or a fecal sample.

235. The method of any one of embodiments 223-234, wherein the disease is cancer.

236. The method of embodiment 235, wherein the cancer is a metastatic cancer.

### EXAMPLES

The application may be better understood by reference to the following non-limiting examples, which is provided as exemplary instances of the application. The following examples are presented in order to more fully illustrate instances and should in no way be construed, however, as limiting the broad scope of the application. While certain instances of the present application have been shown and described herein, it will be obvious that such instances are provided by way of example only. Numerous variations, changes, and substitutions may occur to those skilled in the art without departing from the spirit and scope of the invention. It should be understood that various alternatives to the instances described herein may be employed in practicing the methods described herein.

### Example 1

Cancer samples were purchased from Analytical Biological Services (ABS) biobank. Biospecimens of normal and diseased human tissue in this biobank were collected under stringent requirements for legal compliance with appropriate informed consent for commercial research. Biospecimens include tumor biopsy (archival FFPE) matched to a buffy coat and plasma (cfDNA) from cancer donors. This study evaluated the genetic signature of these samples.

Samples. FFPE, buffy coat, and plasm samples were obtained for Patient 1, a 40 years old female with metastatic adenocarcinoma of colon cancer. The FFPE samples included ˜80% cancer cells, and ˜10-20% fibroblasts and infiltrating mononuclear cells and necrotic tissue (dead tissue).

A plasma sample was obtained for Patient 2, a 69 years old male with metastatic melanoma cancer. The plasma sample from Patient 2 was used as a control to determine the sequencing error rate. The plasma sample was reddish in color, indicating that red and white blood cells during blood draw. Lysed blood cells can cause a higher than expected background non-tumor cfDNA relative to cancer cfDNA (i.e., ctDNA).

Nucleic acid extraction and library preparation. Nucleic acid molecules were extracted from 100 μL of buffy coat (Patient 1) using DNeasy Blood & Tissue Kit or AllPrep® DNA/RNA Kits. Extracted gDNA from both kits was combined, and 1000 ng of the extracted gDNA was used for library construction using Roche KAPA HyperPrep Kits.

Nucleic acid molecules were extracted from a 30 μm slice of FFPE tissue (Patient 1) using DNeasy Blood & Tissue Kit with Xylene or RecoverAll™ Total Nucleic Acid Isolation Kit. 173 ng gDNA extracted from the FFPE sample using the DNeasy Blood & Tissue Kit with Xylene on slides was used for library construction of a first FFPE-based library, and 446 ng gDNA extracted from the FFPE sample using RecoverAll™ Total Nucleic Acid Isolation Kit (without Xylene on slides) was used for library construction of a second FFPE-based library. Libraries were constructed using Roche KAPA HyperPrep Kits followed by 7 cycles of PCR by KAPA HiFi HotStart ReadyMix kit.

Nucleic acid molecules were extracted from 4 mL of plasma (Patient 1 or Patient 2) using MagMAX™ Cell Free Total Nucleic Acid Isolation Kit. 100 ng cfDNA form the Patient 1 plasma sample and 25 ng cfDNA form the Patient 2 plasma sample was used for library construction using Roche KAPA HyperPrep Kits, followed by 7 cycles of PCR by KAPA HiFi HotStart ReadyMix kit.

Accurate quantification of adapter-ligated libraries were done using the KAPA Library Quantification Kit.

Whole genome sequencing. Emulsion PCR and sequencing for each sample was performed using Ultima Genomics instruments and protocols (T-A-C-G flow cycle) in a coverage of ×30-150.

Bioinformatics analysis. 917,319,868 raw reads (Library 1, average length 228 bases at median coverage) were obtained for the buffy coat (Patient 1) sample library (e.g., germline sequences). 2,136,822,000 raw reads (Library 2, average length 183 bases) were obtained for the cfDNA (plasma, Patient 1) sample library. 553,298,760 raw reads (Library 3) (e.g., cfDNA sequences). 1,768,786,851 raw reads (Library 4) (average length of 186 bases) were obtained for the two distinct FFPE-based sequencing libraries (e.g., tumor sequences).

211,8786,000 raw reads (average length 187 bases) were obtained for the cfDNA (plasma, Patient 2) sample library (Library 5).

The raw reads were aligned to the reference genome (hg38) using BWA (version 0.7.15-r1140), and duplicates were marked using Picard Tools (version 2.15.0, Broad Institute) for the buffy coat and FFPE reads or SAM Tools rmdup program for cfDNA reads. After alignment and removing duplicates, the median coverages of the genome were: 45×, 84×, 8×18× and 56× for Libraries 1-5 respectively.

Variants with respect the hg38 reference genome in the FFPE reads were called separately using the HaplotypeCaller program from the GATK4 package (modified to process sequencing data produced by Ultima Genomics instruments and protocols). 4,694,198 variants were called from the first FFPE-based library (Library 3), and 6,702,421 variants were called from the second FFPE-based library (Library 4). The baseline variants from the two FFPE samples were combined for a list of 7,682,808 unique variants (i.e., the “baseline variants”) to account for variances in sample processing, and, for each baseline variant, the number of reads supporting the baseline variant in each of the samples was tabulated. The baseline variants were then filtered to remove germline variants, variants arising from DNA damage due to sample preparation, and variants arising from sequencing errors. First, the baseline variants were filtered to include only SNP variants supported by 2 or more sequencing reads resulting in 4,179,203 unique variants. These variants were then filtered to remove variants from a population database (gnomAD v3, available from the Broad Institute) with allele frequency greater than 0.01 (considered to be likely germline mutations), resulting in 1,292,135 unique variants. These variants were then filtered to remove variants within homopolymer regions of 8 bases or longer, resulting in 1,176,179 unique variants. These variants were then filtered to remove variants that were not supported in complementary strands (suspected of being sequencing errors), resulting in 505,500 unique variants. These variants were then filtered to remove variants detected by reads from the buffy coat sample (presumed germline and/or non-cancerous somatic mutations), resulting in 67,660 unique variants. From the panel of 67,660 unique variants, 17,073 variants present in both FFPE sample libraries and that are expected to induce a cycle shift (i.e., the flowgram signal shifts by one full cycle (e.g., 4 flow positions) or more relative to the reference based on a flow cycle order) were selected for further analysis. As a comparison, 17,509 variants present in both FFPE sample libraries and expected to induce a cycle shift in case of a different flow order (i.e., contains a new zero or new non-zero flowgram signal) were analyzed, as were 5,748 variants that cannot include a cycle shift (i.e., does not contain a new zero or new non-zero flowgram signal).

Bioinformatics analysis was performed using Patient 1 data, with cfDNA from Patient 2 being used to estimate a sequencing error rate against the same set of selected variants. Estimated fraction of cfDNA associated with the cancer in Patient 1, F=Nₜₒₜₐₗ/NᵥₐᵣD, was then determined to be 4.65%, and the background level (E) was determined to be ˜0.35% when cycle shift inducing variants were analyzed. See Table 2. The error corrected fraction, F′=F−E, is therefore ˜4.3%.

TABLE 2

| | | # of reads | # of reads | |
|---|---|---|---|---|
| | # of | mapped to | having | Variants |
| | variants with | variant locus | variants | allele |
| | supporting reads | (NᵥₐᵣD) | (Nₜₒₜₐₗ) | rate |
| Patient 1 FFPE | Nᵥₐᵣ = 17,073 | 574,868 | 158,467 | 27.57% |
| Patient 1 cfDNA | 13,499 | 1,120,053 | 51,956 | 4.64% |
| Control cfDNA | 983 | 767,781 | 2,717 | 0.35% |

When potential cycle shift variants were analyzed, the estimated fraction of cfDNA associated with the cancer in Patient 1 was determined to be 4.34% and the background level was determined to be ˜0.44%, thus providing an error-corrected fraction of 3.9%. See Table 3.

TABLE 3

| | | # of reads | # of reads | |
|---|---|---|---|---|
| | # of | mapped to | having | Variants |
| | variants with | variant locus | variants | allele |
| | supporting reads | (NᵥₐᵣD) | (Nₜₒₜₐₗ) | rate |
| Patient 1 FFPE | Nᵥₐᵣ = 17509 | 563,446 | 147,874 | 26.24% |
| Patient 1 cfDNA | 12996 | 1,116,754 | 48,441 | 4.34% |
| Control cfDNA | 1650 | 765,753 | 3,383 | 0.44% |

When variants that do not induce a cycle shift or potential cycle shift were analyzed, the estimated fraction of cfDNA associated with the cancer in Patient 1 was determined to be 3.92% and the background level was determined to be ˜0.55%, thus providing an error-corrected fraction of 3.37%. See Table 4.

TABLE 4

| | | # of reads | # of reads | |
|---|---|---|---|---|
| | # of | mapped to | having | Variants |
| | variants with | variant locus | variants | allele |
| | supporting reads | (NᵥₐᵣD) | (Nₜₒₜₐₗ) | rate |
| Patient 1 FFPE | Nᵥₐᵣ = 5748 | 189,522 | 45,937 | 24.24% |
| Patient 1 cfDNA | 4037 | 366,954 | 14,389 | 3.92% |
| Control cfDNA | 808 | 251,121 | 1,384 | 0.55% |

### Example 2

The genome of DNA sample NA12878 (sample available from the Coriell Institute for Medical Research) was sequenced using non-terminating, fluorescently labeled nucleotides according to a four flow cycle (T-A-C-G). The sequencing run generated 415,900,002 reads with a mean length of 176 bases. 399,804,925 reads aligned (with BWA, version 0.7.17-r1188) to the hg38 reference genome.

After alignment, reads that perfectly aligned with the reference genome (178,634,625 reads) or reads that contained a single mismatch with the reference genome and aligned with a mapping quality score of 20 or more (27,265,661 reads) were selected. That is, 193,904,639 were excluded for further analysis, for example due to having an indel, multiple mismatches, or potentially incorrect (artifactual) alignment to the reference genome. The 27,265,661 reads were therefore presumed to include true positive NA12878 SNPs, as well as any false positive SNPs that arose from sequencing error. From this pool of 27,265,661 reads, sequencing reads that spanned a mismatched locus more than once were removed to reduce the effect of true positive NA12878 SNPs variants, resulting in a total of 3,413,700 reads containing a mismatch of depth 1).

The remaining 3,413,700 reads each included a mismatch that: (1) was expected to induce a cycle shift if the flowgram flow signal shifts by one full cycle (e.g., 4 flow positions) relative to the reference based on a flow cycle order, (2) potentially could induce cycle shift if a different flow cycle were used (e.g., it generates a new zero or a new non-zero signal in the flowgram), or (3) would not be able to induce a cycle shift regardless of the flow cycle order. Out of 3,413,700 mismatches 1,184,954 (34%) induced a cycle shift, while 1,546,588 (43%) could induce a cycle shift with a different flow order (i.e., “potential cycle shift”). In comparison, theoretical expectation of random mismatches would nominally suggest 42% cycle shift and 46% potential cycle shift mismatches. Overall, the rate of mismatches that induce a cycle shift was 3.7×10⁻⁵ events/base, and the rate of mismatches that induce a potential cycle shift was 4.8×10⁻⁵ events/base. Table 5 shows the 10 most frequent single mismatches that induce a cycle shift and the relative percentages of incidence.

TABLE 5

| Reference | Read | % cases |
|---|---|---|
| TTT | TCT | 7.18 |
| AAA | AGA | 7.18 |
| GAG | GGG | 4.63 |
| CTC | CCC | 4.62 |
| CAG | CGG | 4.12 |
| CTG | CCG | 4.09 |
| AAC | AGC | 3.86 |
| GTT | GCT | 3.83 |
| CAT | CGT | 3.63 |
| GAT | GGT | 3.62 |

The performance of variant calling based on mismatches in each of the three different classes (i.e., those that induce cycle shift, those that potentially induce cycle shift, or those that do not and cannot induce cycle shift) was then evaluated. The reads were aligned to the reference genome with BWA, and variant calling was performed using HaplotypeCaller tool of GATK (version 4). The resulting mismatch calls were filtered by discarding variant calls within a homopolymer longer than 10 bases, or within 10 bases adjacent to a homopolymer having a length 10 bases or more.

The mismatch calls were compared to calls generated for the same NA12878 by the genome-in-the bottle (GIAB) project to determined accuracy #TP/(#FP+#FN+#TP) for each class of mismatches. The sequencing data were randomly down sampled to the indicated mean genomic depth. Mismatches inducing cycle shifts and mismatches potentially inducing cycle shift had higher accuracy that mismatches not inducing cycle shifts, as demonstrated in Table 6.

TABLE 6

| Mismatch type | 30x | 22x | 15x | 8x |
|---|---|---|---|---|
| Cycle shift | 0.9834 | 0.981 | 0.981 | 0.9772 |
| No cycle shift | 0.9799 | 0.9759 | 0.9775 | 0.9696 |
| Potential cycle shift | 0.9826 | 0.9808 | 0.9795 | 0.9767 |

### Example 3

Five matched tumor, buffy coat, and cfDNA samples were analyzed in this example, as detailed in Table 7.

TABLE 7

| Identifier | Age | Gender | Cancer Type |
|---|---|---|---|
| ABS1405-20 | 40 | Female | Colon Cancer |
| ABS1908-13 | 69 | Male | Melanoma |
| ABS1212-24 | 71 | Male | Colon Cancer |
| 333_CRCs_30 | 50 | Female | Colon Cancer |
| 333_LuNgh_85 | 56 | Female | Lung Cancer |

Sequencing was carried out using in-house sequencers using a flow-sequencing method. Raw sequencing reads were aligned to a reference genome (hg38) using BWA (version 0.7.15-r1140), and duplicates were marked using Picard tools (version 2.15.0) and removed. cfDNA samples were sequenced with a coverage of 75-100× after alignment and duplication removal, FFPE samples with a coverage of 75-120× and buffy coat samples with a coverage of 35-55×.

Variants from the FFPE samples were called separately using HaplotypeCaller program from the GATK4 package with specific modifications to the error model to adapt it to the error properties of the sequencing data (see e.g., FIGS. 13A and 13B). Detected haplotypes were extracted from the GATK outputs as well as the variant calling VCF file. The VCF output of each FFPE sample was filtered as follows to generate an initials small nucleotide variant panel: (1) Initial variants from FFPE sample were called; (2) variants other than SNPs were excluded; (3) variants appearing in gnomAD (Karczewski et al., The mutational constraint spectrum quantified from variation in 141,456 humans, Nature vol. 581, pp. 434-443 (2020)) with an allele frequency greater than 0.01 were excluded; (4) variants with multiple alternative alleles were excluded; and (5) variants in low complexity regions (LCRs) of the genome were excluded.

Sequencing reads from the FFPE, cfDNA, and buffy coat samples were then evaluated for quality. Sequencing reads that had a likelihood of correct call at each flow during the sequencing were excluded. For the remaining reads, the likelihood of the read as supporting a reference allele or an alternative allele was determined, and sequencing reads with less than a three order magnitude difference in likelihood between supporting the reference allele and the alternative allele were excluded.

The small nucleotide variant panel was further filtered according to the following: (1) small nucleotide variants with 0 coverage in the buffy coat sample were excluded; (2) small nucleotide variants with 1 or more supporting reads from the buffy coat sample were excluded; (3) variants with a non-negligible likelihood of being a germline variant were excluded, as determined by calculating the likelihood of the measured number of reads supporting the tumor sequence in the cfDNA and in the buffy coat samples given an allele fraction of 0.5 (e.g., null hypothesis), and excluding the variant if the null hypothesis cannot be rejected with a p-value of 10⁻³; (4) small nucleotide variants with a non-negligible amount of low mapping quality sequence reads (at least 95% of sequence reads mapped with a mapping quality score of 60 form the BWA aligner) were excluded; (5) small nucleotide variant at loci with a bias in likelihood between reference and alternative alleles were excluded; (6) small nucleotide variants with a low tumor allele fraction (e.g., less than 5%) in the FFPE sample were excluded: and (7) small nucleotide variants with a high tumor allele fraction (e.g., greater than 40%) in the FFPE sample were excluded.

Exclusion of small nucleotide variants to form the small nucleotide variant panel at different stages of the variant filtering funnel is shown in Table 8, where each entry indicates the number of small nucleotide variants that passed the respective filtering stage. In some implementations, the filtering stages listed in Table 8 may be performed is a different order. For example, variants with non-zero germline coverage may be removed via filtering prior to removing LCRs via filtering.

TABLE 8

| Stage \| Sample | 333_CRCs_30 | ABS1212-24 | ABS1405-20 | ABS1908-13 | 333_LuNgh_85 |
|---|---|---|---|---|---|
| small nucleotide | 642003 | 549882 | 534112 | 514773 | 537996 |
| variants, not in | | | | | |
| gnomAD | | | | | |
| Discard multiple | 560037 | 479675 | 422424 | 403556 | 474030 |
| alternative alleles | | | | | |
| Discard LCRs | 519864 | 447953 | 395590 | 379947 | 442425 |
| Nonzero germline | 491218 | 435188 | 378687 | 358819 | 426428 |
| coverage | | | | | |
| 0 germline reads | 98100 | 47943 | 35081 | 10569 | 13076 |
| Low likelihood | 82171 | 39398 | 25699 | 3984 | 5494 |
| of being germline | | | | | |
| No loci with low | 12224 | 5105 | 18100 | 2764 | 1893 |
| mapping quality | | | | | |
| reads | | | | | |
| No likelihood | 8566 | 3176 | 17479 | 2548 | 1088 |
| reference bias | | | | | |
| In tumor fraction | 7895 | 2936 | 12797 | 1232 | 886 |
| range | | | | | |

The sequencing reads from the cfDNA sample that correspond with the filtered small nucleotide variants were filtered to retain high confidence sequencing reads. For each sequencing read, a likelihood of supporting a reference allele and a likelihood of supporting an alternative allele was determined, and any sequencing read with less than a 10 magnitude difference in these likelihoods was excluded from downstream analysis.

Using the remaining cfDNA sequencing reads, a locus-specific tumor fraction was determined for all loci corresponding to the filtered set of small nucleotide variants. The likelihood of the measured tumor fraction in each small nucleotide variant locus was calculated, and loci with a likelihood lower than a p-value of 10⁻³ were discarded. This process was repeated until no loci were discarded.

Initial Tumor Fraction Detection. For each cfDNA sample, the tumor fraction was determined using reads that support the alternative allele and reference allele. For each case sample, the four other samples served as a control sample. Control tumor fractions were determined using two different methods: (1) case cfDNA vs. control signature, and (2) control cfDNA vs. case signature. In the first mode, the somatic mutation patterns measured for control FFPE samples was used, and the tumor fraction for the case cfDNA was determined. In the second mode, cfDNA from control samples was tested against the case signature, and the tumor fractions for the control samples were determined.

In order to use a cfDNA sample as a control for the signature of another, the signature under consideration was filtered to exclude any small nucleotide variant that might introduce artifacts. For each combination of signature from patient A and cfDNA from patient B, any small nucleotide variant in the signature for patient A with a non-negligible likelihood of being a variant of patient B (germline variants of patient A were already filtered as described in the previous section) was excluded. Also excluded were small nucleotide variants in signature A that also appear in the small nucleotide variant signature of patient B, regardless of quality or other features of the variant.

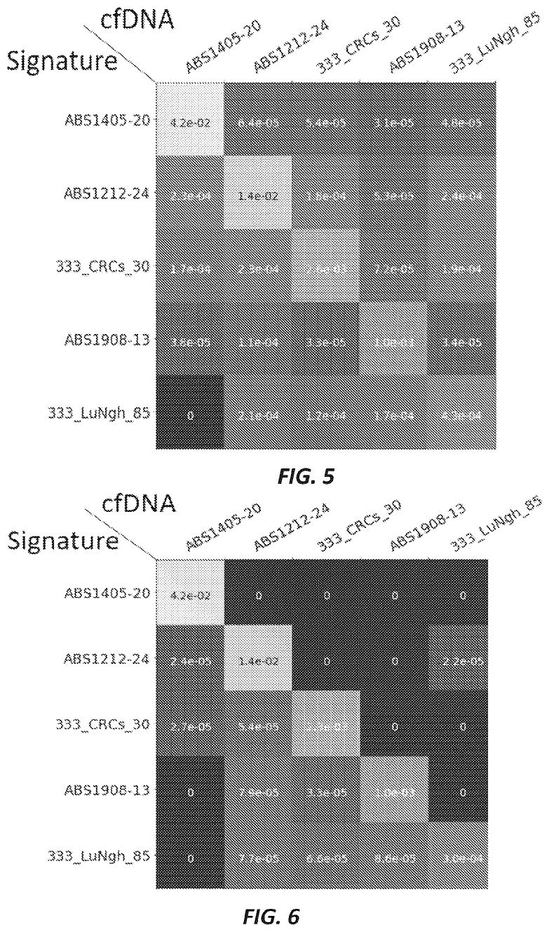

The tumor fractions for the case and controls in all signature and cfDNA combinations was measured (25 combinations, see FIG. 5). Measured case tumor fraction were in the range of 4.3×10⁻⁴ to 4.2×10⁻², spanning a high dynamic range. In all but the lowest tumor fractions, a signal separated from the background tumor fraction, which is up to 1.7×10⁻⁴, was clearly observed. However, the background was relatively inconsistent, which limits the sensitivity of the measurement at low tumor fractions.

Background reduction using motif-level information. Tumor fraction estimation was improved and background levels reduced by considering additional features of the reads. Each read supporting the alternative allele was labeled by its first-order motif, namely its base and small nucleotide variant type (e.g., TGC to TAC, a variant where G mutates into A). A variant fraction was measured on a per-motif basis for all sequencing reads matching the alternative allele of control signatures, which indicated a background signal per patient. That background signal distribution constitutes the sum of all mechanisms that can produce a background signal in the measurement, such as biological noise (e.g., somatic mutation in blood cells or elsewhere), errors introduced in library preparation, and errors introduced in sequencing. It was discovered that the background signal distribution was highly non-uniform across motifs. Since the distribution of signal coming from a true tumor fraction is expected to be uniform (cfDNA originating from the tumor is equally likely to be detected regardless of motif), the background distribution provides information that allows a clearer separation of signal a background. The background signal distribution per cfDNA sample was measured for all 5 combinations of said cfDNA sample from one patient with signatures from other patients. Additionally, the calculation was repeated for the remaining controls, with one modification—the signature used as a control each time was removed from the background distribution to avoid artifacts.

Two approaches to the estimation of tumor fraction given a measured background distribution were evaluated.

Background reduction using motif-level information—MLE method. In the first approach, the measurement of reference and alternative allele sequencing reads were modeled within each motif as a binomial distribution with probability pₘ=TF+BGₘ, where pₘ is the probability of observing a read supporting the alternative allele in motif m, TF is the tumor fraction which is constant across motifs, and BGₘ is the measured background distribution for motif m. For a given estimated tumor fraction, the likelihood of the measured result per motif was measured, and sum of the log-likelihoods across all motifs was determined. This total log-likelihood value was then maximized using standard optimization methods to find the tumor fraction that yields the highest log-likelihood. This Maximum Likelihood Estimate (MLE) for TF yields a lower background by over 10-fold, with about half the controls yielding a value of 0. See FIG. 6.

Background reduction using motif-level information—Fisher method. This Fisher Exact Test yields a likelihood for two sets of measurement from two groups to have arisen from the same distribution. In order to apply this test for each motif given some tumor fraction to obtain a likelihood estimation, a Monte Carlo approach was taken. For each motif, a 2×2 contingency table was constructed, containing a number of sequencing reads supporting the alternative and the reference allele (columns), in the control samples (i.e., the background distribution) and the considered case cfDNA (rows).

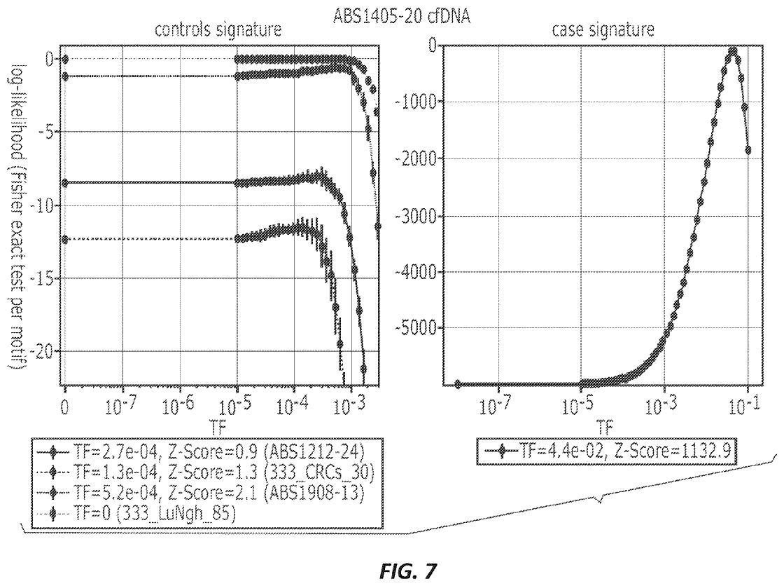

The control sequencing read distribution result from some unknown background distribution, while the case sequencing read distribution results arose from the same background distribution plus some unknown tumor fraction. In order to test the likelihood of a specific estimated tumor fraction, a random binomial sample from the background distribution was simulated using a probability of the binomial distribution equal to the estimated tumor fraction. Sequencing reads were moved from the reference allele column to the alternative allele column according to the simulated binomial distribution for the given estimated tumor fraction. This process was performed iteratively for a range of estimated tumor fraction values. A Fisher Exact Test was the applied to obtain a likelihood per motif for each estimated tumor fraction (e.g., in the range of estimated tumor fraction values). The log-likelihoods for all variant motifs were summed to obtain an estimated total log-likelihood. This total log-likelihood value was then maximized using standard optimization methods to find the tumor fraction that yields the highest log-likelihood. The log-likelihood profiles for a plurality of estimated tumor fractions are shown in FIG. 7. In FIG. 7, the example estimated tumor fractions (TF) that were evaluated were 0, 5.2e⁻⁴, 1.3e⁻⁴, and 2.7e⁻⁴.

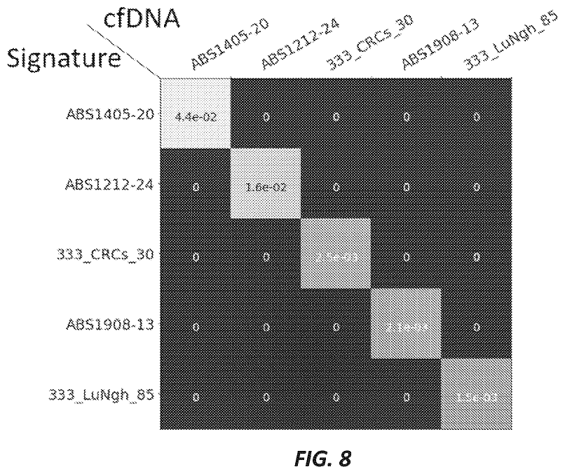

Additionally, this method allows the significance of the measured tumor fraction to be determined. The standard deviation of the log-likelihood value at the optimal tumor fraction was measured by generating multiple realizations of the Monte Carlo model. The log-likelihood value at TF=0 was subtracted from the log-likelihood value at the optimal TF (e.g., 4.4e⁻²), and this difference was divided by the standard deviation, yielding a Z-Score. This Z-Score value can be used to differentiate between significant and non-significant measurements of TF. A significance threshold of Z-Score=10 was set, and any fractions in the control samples with a lower Z-score was set to 0, as those results would not be called as detections. See FIG. 8. Interestingly, the Fisher method estimation for TF yielded a background level of 0 for all 20 controls. This result underscores the incredible potential in the usage of the motif-level information to estimate cfDNA tumor samples accurately.

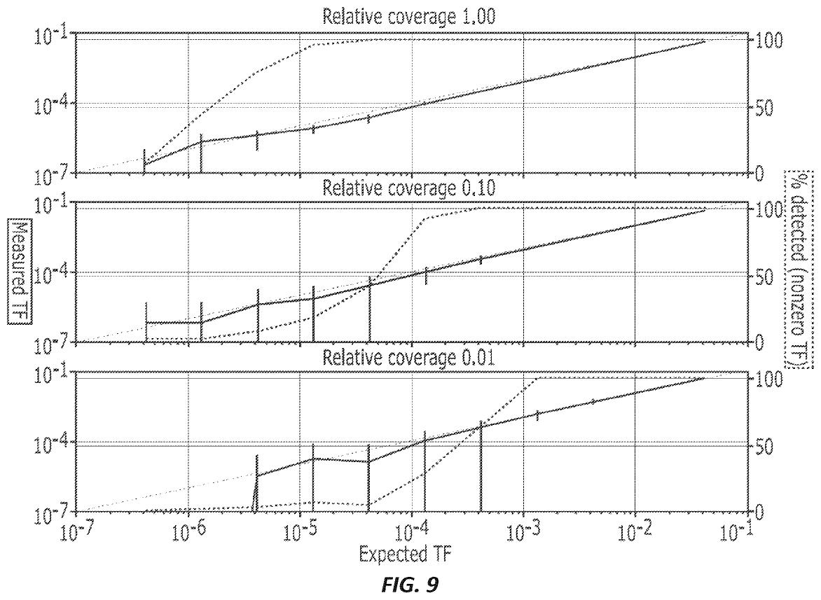

Estimating detection limit by down-sampling simulations. Since the background level can be determined as 0 for the sample under consideration, the sensitivity of the method is not limited by background mutations and sequencing errors. However, the remaining limitation is the effective coverage of cfDNA sample in the signature under consideration. In order to be able to detect low TF values, a sufficiently large number of reads must be collected. To investigate the effective detection limit of our method, random realization of downsampled data from patient ABS1405-20 was generated (FIG. 9). 50 realizations of downsamples of the reads supporting the alternative allele in the downsampling ratio range of 10⁻⁵−1 were generated, and the mean and standard deviation of the measured TF was calculated (FIG. 9, top panel, left axis). Additionally, the frequency where a non-zero value was measured was determined, namely the detection probability (FIG. 9, top panel, right axis). It was found that at the current effective coverage of 316967 reads in this patient's signature (after read filtering), it was possible to detect a signal 50% of the time at TF₅₀≈1.5×10⁻⁶. Random 0.1× and 0.01× downsamples of the total number of reads (supporting both reference and alternative allele) were generated, and it was found that the TF₅₀ were reduced to 4×10⁻⁵ and 3×10⁻⁴ respectively (FIG. 9, middle panel and bottom panel).

### Example 4

A FeatureMap tool is used to evaluate somatic variant calling and tumor fraction determination (see e.g., FIG. 10). As can be seen by comparing FIG. 10 with FIG. 11, the FeatureMap tool, in some implementations, requires fewer steps than the HaplotypeCaller tool. FeatureMap considers only small nucleotide variant motifs, where each small nucleotide variant is evaluated independently (e.g., in contrast to haplotype variants which can encompass multiple small nucleotide variants).

In the exemplary FeatureMap tool, small nucleotide variants are stored in a VCF file 1010 (e.g., in contrast to TSV or CSV files containing reference and alternative haplotype likelihoods, as described above with regards to Example 3).

Using only cycle skip small nucleotide variants extracted from FeatureMaps, the number of reads that match each somatic small nucleotide variant signature are counted, and these values are divided by the coverage (e.g., the total number of reads covering each cycle skip motif) for each small nucleotide variant. In some instances, one or more filtration steps are used to exclude one or more reads from analysis, as explained elsewhere herein.

Typically, reads are initially filtered for MapQ=60. Each substitution (e.g., small nucleotide variant) is then required to match the reference sequence in the adjacent −/+5 bases (e.g., there must be alignment across 5 bases upstream and 5 bases downstream of the small nucleotide variant). This initial filters typically retains approximately 80-85% of the substitutions.

An exemplary comparison of the results from the HaplotypeCaller and the FeatureMap tools is provided. Reads for the all-variant analysis are filtered to a subset of reads (e.g., to exclude reads of lower quality). An additional analysis filtered out C:G to T:A motifs from the set of all variants. This is because C:G to T:A motifs typically exhibit increased background levels. In addition, background levels are in the 1-5*10⁻⁵ range when the C:G->T:A motifs are excluded.

Substitution error rates. For each cfDNA signature, FeatureMaps can be used to calculate the error rate across the entire genome. Cycle skip motifs typically have much lower error rates. This is due to the fact that cycle skip motifs exhibit virtually no sequencing errors. An exception to this is C->T cycle skip motifs; these motifs generally have higher error rates than other non-cycle skip motifs (e.g. the cycle skip motif ACG->ATG typically has a 100-fold higher error rate than the non-cycle skip TCC->TGC).

Coverage values for each motif are calculated directly from the sample sequencing BAM files. Only reads with a MapQ=60 are retained for analysis. The number of reads per trinucleotide motif is calculated in order to median coverage. The coverage values are further corrected for the read filtering ratio (e.g., for how many reads are retained after filtering). For example, when considering cycle-skip variants coverage can be calculated as the number of cycle skip motifs multiplied by the median coverage.

What is claimed is:

1. A method of determining a level of a disease in an individual, comprising:
obtaining sequencing data for nucleic acid molecules obtained from a fluidic sample from the individual, the sequencing data comprising sequencing reads associated with loci selected from a personalized disease-associated small nucleotide variant (SNV) panel;
generating, using the sequencing data, a plurality of variant motif-specific models that each associate sequencing data corresponding to a respective variant motif, a background factor indicative of a false positive error rate for the respective variant motif, and an estimated fraction of the nucleic acid molecules associated with the disease; and
determining, from the plurality of variant motif-specific models, a fraction of the nucleic acid molecules associated with the disease for the individual, wherein the fraction indicates the level of the disease in the individual.

2. The method of claim 1, wherein the level of disease in the individual is a presence or absence of the disease.

3. The method of claim 1, wherein the level of disease in the individual is a quantitative value indicating the severity of the disease.

4. A method of determining a presence or absence of a disease in an individual, comprising:
obtaining sequencing data for nucleic acid molecules obtained from a fluidic sample from the individual, the sequencing data comprising sequencing reads associated with loci selected from a personalized disease-associated small nucleotide variant (SNV) panel;
generating, using the sequencing data, a plurality of variant motif-specific models that each associate sequencing data corresponding to a respective variant motif, a background factor indicative of a false positive error rate for the respective variant motif, and an estimated fraction of the nucleic acid molecules associated with the disease; and
determining, from the plurality of variant motif-specific models, a fraction of the nucleic acid molecules associated with the disease for the individual; and
comparing the fraction to a background level, wherein the fraction being above the background level indicates the presence of the disease in the individual.

5. The method of any one of claims 1-4, wherein the sequencing data is generated by sequencing the nucleic acid molecules using non-terminating nucleotides provided in separate nucleotide flows according to a flow-cycle order comprising a plurality of flow positions, wherein the flow positions correspond to the nucleotide flows.

6. The method of any one of claims 1-4, wherein obtaining the sequencing data comprises sequencing the nucleic acid molecules using non-terminating nucleotides provided in separate nucleotide flows according to a flow-cycle order comprising a plurality of flow positions, wherein the flow positions correspond to the nucleotide flows.

7. The method of any one of claims 1-6, further comprising:
for each of the sequencing reads, determining a likelihood that the sequencing read corresponds to a variant sequence and a likelihood that the sequencing read corresponds to a reference sequence, and
for a respective sequence read, if the difference between the likelihood that the respective sequencing read corresponds to the variant sequence and the likelihood that the respective sequencing read corresponds to a reference sequence is less than a predetermined likelihood difference threshold, then excluding sequencing data corresponding to the respective sequencing read from the plurality of variant motif-specific models.

8. The method of claim 7, wherein the variant sequence and the reference sequence are corresponding haplotype sequences.

9. The method of claim 7 or 8, wherein the variant sequence and the reference sequence differ by at least two bases.

10. The method of claim 7 or 8, wherein the variant sequence and the reference sequence comprise at least two loci from the personalized disease-associated SNV panel.

11. The method of any one of claims 1-6, further comprising, for each of the sequencing reads:
identifying a variant locus from the personalized disease-associated SNV panel within the sequencing read, wherein the variant locus is associated with a single base variant;
trimming the sequencing read to generate a trimmed sequencing read comprising the variant locus and excluding any other variant locus from the personalized disease-associated SNV panel;
determining a likelihood that the trimmed sequencing read corresponds to a variant sequence comprising and a likelihood that the sequencing read corresponds to a reference sequence, wherein the variant sequence and the reference sequence each comprises the variant locus and excludes any other variant locus from the personalized disease-associated SNV panel; and
for a respective trimmed sequencing read, if the difference between the likelihood that the respective trimmed sequencing read corresponds to the variant sequence and the likelihood that the respective trimmed sequencing read corresponds to a reference sequence is less than a predetermined likelihood difference threshold, then excluding sequencing data corresponding to the respective trimmed sequencing read from the plurality of variant motif-specific models.

12. The method of any one of claims 7-11, wherein the predetermined likelihood difference threshold is set at a value of 5 orders of magnitude or higher.

13. The method of any one of claims 1-12, wherein at least 90% of SNVs in the personalized disease-associated SNV panel are associated with SNV sequencing data that differs from reference sequencing data associated with a reference sequence at two or more flow positions, wherein the SNV sequencing data and the reference sequencing data are sequenced using non-terminating nucleotides provided in separate nucleotide flows according to a flow-cycle order comprising a plurality of flow positions, wherein the flow positions correspond to nucleotide flows.

14. The method of any one of claims 1-13, wherein at least 90% of SNVs in the personalized disease-associated SNV panel are associated with SNV sequencing data that differs from reference sequencing data associated with a reference sequence across one or more flow cycles in a flow-cycle order, wherein the sequencing data and the reference sequencing data are sequenced using non-terminating nucleotides provided in separate nucleotide flows according to the flow-cycle order.

15. The method of any one of claims 1-14, further comprising characterizing the sequencing reads as an alternate read, a reference read, or an ambiguous read, wherein a sequencing read characterized as an ambiguous read is excluded from the plurality of variant motif-specific models.

16. The method of any one of claims 1-15, wherein the plurality of variant motif-specific models comprises a respective variant motif-specific model for each of a plurality of trinucleotide SNP motifs.

17. The method of claim 16, wherein the plurality of variant motif-specific models comprises 192 trinucleotide SNP variant motif-specific models.

18. The method of any one of claims 1-17, wherein each variant motif-specific model associates the sequencing data corresponding to variant motif, m, to the background factor, BGₘ, and the estimated faction, F, according to:
Nₘ^{alt}=(F+BGₘ)Nₘ^{total},
wherein Nₘ^{alt} is a number of alternative sequencing reads comprising a locus corresponding to variant motif m and Nₘ^{total} is a total number of sequencing reads comprising a locus corresponding to variant motif m.

19. The method of any one of claims 1-18, wherein each variant motif-specific model is a binomial distribution of the sequencing reads comprising a locus corresponding to variant motif m, with a probability, pₘ, of observing an alternative sequencing read comprising a locus corresponding to variant motif m based on pₘ=F+BGₘ, wherein F is the estimated fraction, and BGₘ is the background factor.

20. The method of any one of claims 1-19, wherein determining the fraction for the individual comprises determining a maximum likelihood estimate for the fraction given the plurality of variant motif-specific models.

21. The method of any one of claims 1-20, wherein determining the fraction for the individual comprises:
determining, for each variant motif-specific model, a statistical value indicative of a likelihood of each of a plurality of estimated fractions, given the sequencing data for the nucleic acid molecules obtained from the fluidic sample from the individual corresponding to the respective variant motif, and
determining a most likely fraction given the statistical values for each variant motif.

22. The method of claim 21, wherein each variant motif-specific model comprises a plurality of binomial distributions of sequencing reads comprising a locus corresponding to variant motif m, with each binomial distribution having a probability of a sequencing read being an alternate read equal to an estimated fraction selected from the plurality of estimated fractions.

23. The method of any one of claims 1-18, wherein determining the fraction for the individual comprises:
determining, for each variant motif-specific model, a statistical value indicative of a likelihood of each of a plurality of estimated fractions, given the sequencing data for the nucleic acid molecules obtained from the fluidic sample from the individual corresponding to the respective variant motif and control sequencing data for nucleic acid molecules obtained from one or more control fluidic samples corresponding to the respective variant motif, wherein the control sequencing data is adjusted for one or more non-zero estimated fractions; and
determining a most likely fraction given the statistical values for each variant motif.

24. The method of claim 23, wherein the control sequencing data is adjusted for each of the one or more non-zero estimated fractions using a random realization method with a distribution probability equal to the respective non-zero estimated fraction.

25. The method of claim 24, wherein, for each of a non-zero estimated tumor fraction, the statistical value indicative of the likelihood is an average of a plurality of likelihood values obtained using a plurality of random realizations, each with a distribution probability equal to the respective non-zero estimated fraction.

26. The method of any one of claims 23-25, wherein the one or more control fluidic samples comprises a plurality of control fluidic samples.

27. The method of any one of claims 21-26, wherein each statistical value is determined using an exact test.

28. The method of claim 27, wherein each statistical value is determined using Fisher's exact test.

29. The method of any one of claims 1-28, further comprising determining whether the difference between the fraction for the individual is greater than a background level with statistical significance.

30. The method of any one of claims 1-29, wherein the fraction for the individual is a tumor fraction.

31. The method of any one of claims 1-30, wherein the nucleic acid molecules are cell-free DNA (cfDNA) molecules.

32. The method of any one of claims 1-31, further comprising generating the personalized disease-associated SNV panel.

33. The method of claim 32, wherein the personalized disease-associated SNV panel comprises SNVs detected from sequencing data for nucleic acid molecules derived from a diseased tissue sample.

34. The method of claim 33, wherein the sample of the diseased tissue is a tumor biopsy sample obtained from the individual.

35. The method of any one of claims 1-34, further comprising excluding, from the personalized disease-associated SNV panel, SNVs other than single nucleotide polymorphisms (SNPs).

36. The method of any one of claims 1-35, further comprising excluding, from the personalized disease-associated SNV panel, SNVs present in a general population of individuals at an allele frequency greater than a predetermined allele threshold.

37. The method of claim 36, wherein the predetermined allele threshold is about 0.01.

38. The method of any one of claims 1-37, further comprising excluding, from the personalized disease-associated SNV panel, SNVs at loci with two or more non-reference alleles.

39. The method of any one of claims 1-38, further comprising excluding, from the personalized disease-associated SNV panel, SNVs within a low complexity region.

40. The method of any one of claims 1-39, further comprising excluding, from the personalized disease-associate SNV panel, SNVs characterized as likely germline variants or likely non-disease related somatic variants.

41. The method of claim 40, wherein the SNVs characterized as likely germline variants or as likely non-disease related somatic variants are characterized by sequencing nucleic acid molecules derived from a sample of non-diseased tissue obtained from the individual.

42. The method of any one of claims 1-41, wherein nucleic acid molecules derived from a sample of non-diseased tissue obtained from the individual are sequenced to obtain non-diseased tissue sequencing data, and the method further comprises excluding, from the personalized disease-associate SNV panel, SNVs at loci that have no sequencing coverage within the non-diseased tissue sequencing data.

43. The method of claim 41 or 42, wherein the sample of non-diseased tissue comprises white blood cells.

44. The method of claim 41 or 42, wherein the sample of non-diseased tissue comprises peripheral blood mononuclear cells.

45. The method of any one of claims 41-44, wherein the sample of non-diseased tissue is a buffy coat.

46. The method of any one of claims 1-45, further comprising excluding, from the personalized disease-associate SNV panel, SNVs at loci associated with a predetermined number or proportion of sequencing reads that have a mapping quality score below a predetermined mapping quality threshold.

47. The method of any one of claims 1-46, further comprising excluding, from the personalized disease-associate SNV panel, SNVs at loci that have a bias for reference reads or alternate reads.

48. The method of any one of claims 1-47, wherein nucleic acid molecules derived from a diseased tissue sample obtained from the individual are sequenced to obtain diseased tissue sequencing data, and the method further comprises excluding, from the personalized disease-associate SNV panel, SNVs that have a variant allele fraction in the nucleic acid molecules derived from the diseased tissue sample lower than a predetermined low-fraction threshold.

49. The method of any one of claims 1-48, wherein nucleic acid molecules derived from a diseased tissue sample obtained from the individual are sequenced to obtain diseased tissue sequencing data, and the method further comprises excluding, from the personalized disease-associate SNV panel, SNVs that have a variant allele fraction in the nucleic acid molecules derived from the diseased tissue sample higher than a predetermined high-fraction threshold.

50. The method of any one of claims 1-49, further comprising identifying one or more outlier SNVs within the personalized disease-associated SNV panel that are associated with a locus-specific fraction outlier, given the sequencing data for nucleic acid molecules obtained from a fluidic sample from the individual, and excluding sequencing data associated with said one or more outlier SNVs from the plurality of variant motif-specific models.

51. The method of any one of claims 1-50, wherein the method further comprises measuring a recurrence of the disease.

52. The method of any one of claims 1-51, wherein the method further comprises measuring a progression or regression of the disease by comparing the level of the disease to a previously measured level of the disease.

53. The method of claim 52, wherein progression or regression of the disease is based on a statistically significant change in the measured level of the disease compared to the previously measured level of the disease.

54. The method of any one of claims 1-53, wherein the fluidic sample is a blood sample, a plasma sample, a saliva sample, a urine sample, or a fecal sample.

55. The method of any one of claims 1-54, wherein the disease is cancer.

56. The method of claim 55, wherein the cancer is a metastatic cancer.

57. The method of any one of claims 1-56, wherein the sequencing data is untargeted sequencing data.

58. The method of claim 57, wherein the sequencing data is obtained from an untargeted whole genome.

59. The method of any one of claims 1-58, wherein the mean sequencing depth of the sequencing data is at least 0.01.

60. The method of any one of claims 1-59, wherein the mean sequencing depth of the sequencing data is less than about 100.

61. The method of any one of claims 1-60, wherein the mean sequencing depth of the sequencing data is less than about 10.

62. The method of any one of claims 1-61, wherein the mean sequencing depth of the sequencing data is less than about 1.

63. The method of any one of claims 1-62, wherein the personalized disease-associated SNV panel comprises passenger mutations.

64. The method of any one of claims 1-63, wherein the personalized disease-associated SNV panel comprises driver mutations.

65. The method of any one of claims 1-64, wherein the selected loci from the personalized disease-associated SNV panel comprise about 300 or more SNV loci.

66. The method of any one of claims 1-65, wherein the sequencing data is obtained using surface-based sequencing of nucleic acid molecules, and wherein the nucleic acid molecules are not amplified prior to attaching the nucleic acid molecules to a surface.

67. The method of any one of claims 1-66, wherein the sequencing data is obtained without using unique molecular identifiers (UMIs).

68. The method of any one of claims 1-67, wherein the sequencing data is obtained without using sample identification barcodes.

69. The method of any one of claims 1-68, wherein the background factor is based on sequencing data for nucleic acid molecules obtained from a plurality of control individuals.

70. The method of claim 69, wherein the sequencing data for nucleic acid molecules obtained from a plurality of control individuals comprises sequencing reads associated with loci selected from the personalized disease-associated SNV panel.

71. The method of claim 69 or 70, wherein the sequencing data for the nucleic acid molecules of the individual and the sequencing data for the nucleic acid molecules for the plurality of control individuals are simultaneously obtained in a pooled sample.

72. The method of any one of claims 1-71, further comprising generating a report that indicates the presence, absence, or level of disease in the individual.

73. The method of claim 72, further comprising providing the report to a patient or a healthcare representative of the patient.

74. A system, comprising:
one or more processors; and
a non-transitory computer-readable storage medium that stores one or more programs comprising instructions for implementing the method of any one of claims 1-73.

75. A method, comprising:
generating a sequencing read by sequencing a nucleic acid molecule using non-terminating nucleotides provided in separate nucleotide flows according to a flow-cycle order, wherein the sequencing read comprises a plurality of flow positions that correspond to the nucleotide flows;
identifying, within the sequencing read, a variant locus for a single base variant within a disease-associated small nucleotide variant (SNV) panel;
trimming the sequencing read to generate a trimmed sequencing read comprising the variant locus and excluding any other variant locus from the disease-associated SNV panel;
determining a likelihood that the trimmed sequencing read corresponds to a variant sequence comprising and a likelihood that the sequencing read corresponds to a reference sequence, wherein the variant sequence and the reference sequence each comprises the variant locus and excludes any other variant locus from the disease-associated SNV panel; and
calling the sequencing read, based on the likelihoods, as supporting the presence of a variant at the variant locus, not supporting the presence of a variant at the variant locus, or ambiguous.

76. The method of claim 75, wherein the trimmed sequencing read comprises 15 or fewer flow positions.

77. The method of claim 75 or 76, wherein the length of the trimmed sequencing read is based on the likelihoods.

78. The method of any one of claims 75-77, wherein the disease-associated SNV panel is a personalized disease-associated SNV panel.

79. The method of any one of claims 75-78, wherein the variant sequence is associated with a tumor genome and the reference sequence is associated with a non-tumor genome.

80. The method of any one of claims 75-79, wherein the method is performed for a plurality of sequencing reads, wherein at least a portion of the sequencing reads in the plurality of sequencing reads comprise different variant loci.

81. The method of claim 80, wherein the method further comprises determining a fraction of the nucleic acid molecules associated with the disease for the individual, wherein the fraction indicates the level of the disease in the individual a level of disease in an individual.

82. The method of claim 81, wherein the level of disease in the individual is a presence or absence of the disease.

83. The method of claim 81, wherein the level of disease in the individual is a quantitative value indicating the severity of the disease.

84. The method of any one of claims 81-83, wherein the fraction is a tumor fraction.

85. The method of any one of claims 75-84, wherein nucleic acid molecule is obtained from a fluidic sample from an individual.

86. The method of claim 85, wherein the fluidic sample is a blood sample, a plasma sample, a saliva sample, a urine sample, or a fecal sample.

87. The method of any one of claims 75-86, wherein the disease is cancer.

88. The method of claim 87, wherein the cancer is a metastatic cancer.
